SEQUENCE LISTING (24 sequences)

SEQ ID NO: 1 - DNA, 24 bp, Artificial Sequence
  Forward primer for amplification of ORF for Aurora kinase
tcaaaaaagaggcagtgggctttg

SEQ ID NO: 2 - DNA, 21 bp, Artificial Sequence
  Reverse primer for amplication of ORF for Aurora kinase
ctgaatttgctgtgatccagg

SEQ ID NO: 3 - DNA, 46 bp, Artificial Sequence
  Synthetic construct - sequence inserted into NdeI site of vector pSB2-TOPO
cataatgggccatcatcatcatcatcacggtggtcatatgtccctt

SEQ ID NO: 4 - DNA, 27 bp, Artificial Sequence
  Synthetic construct - sequence inserted into BamHI site of vector pSB2-TOPO
aagggggatcctaaactgcagagatcc

SEQ ID NO: 5 - DNA, 60 bp, Artificial Sequence
  Synthetic construct - portion of resulting plasmid sequence
aaggaggagatatacataatgggccatcatcatcatcatcacggtggtcatatgtccctt

SEQ ID NO: 6 - DNA, 27 bp, Artificial Sequence
  Synthetic construct - portion of resulting plasmid sequence after ORF
aagggggatcctaaactgcagagatcc

SEQ ID NO: 7 - protein, 14 aa, Artificial Sequence
  N-terminal end of expressed Aurora kinase
MGHHHHHHGGHMSL

SEQ ID NO: 8 - protein, 4 aa, Artificial Sequence
  Synthetic construct - C-terminal end of expressed Aurora kinase
EGGS

SEQ ID NO: 9 - DNA, 51 bp, Artificial Sequence
  Forward primer (PPfor) for amplifying phosphatase gene
gcagagatccgaattcgagctccgtcgacggatggagtgaaagagatgcgc

SEQ ID NO: 10 - DNA, 55 bp, Artificial Sequence
  Reverse primer (PPrev) for amplifying phosphatase gene
ggtggtggtgctcgagtgcggccgcaagctttcatcatgcgccttctccctgtac

SEQ ID NO: 11 - DNA, 19 bp, Artificial Sequence
  Forward primer for amplifying ORF for c-Abl
gacaagtgggaaatggagc

SEQ ID NO: 12 - DNA, 19 bp, Artificial Sequence
  Reverse primer for amplifying ORF for c-Abl
cgcctcgtttccccagctc

SEQ ID NO: 13 - DNA, 12 bp, Artificial Sequence
  Synthetic construct - sequence inserted into NdeI site of vector pSGX3-TOPO
catatgtccctt

SEQ ID NO: 14 - DNA, 29 bp, Artificial Sequence
  Synthetic construct - sequence inserted into BamHI site of vector pSGX3-TOPO
aagggcatcatcaccatcaccactgatcc

SEQ ID NO: 15 - DNA, 26 bp, Artificial Sequence
  Synthetic construct - portion of resulting plasmid sequence
aaggaggagatatacatatgtccctt

SEQ ID NO: 16 - DNA, 29 bp, Artificial Sequence
  Synthetic construct - portion of resulting plasmid sequence after ORF
aagggcatcatcaccatcaccactgatcc

SEQ ID NO: 17 - protein, 8 aa, Artificial Sequence
  Synthetic construct - C-terminal end of expressed c-Abl
EGHHHHHH

SEQ ID NO: 18 - DNA, 39 bp, Artificial Sequence
  Mm05582dS4 oligonucleotide used to prepare plasmid for Abl T315I
ccaccattctacataatcattgagttcatgacctatggg

SEQ ID NO: 19 - DNA, 39 bp, Artificial Sequence
  Mm05582dA4 oligonucleotide used to prepare plasmid for Abl T315I
cccataggtcatgaactcaatgattatgtagaatggtgg

SEQ ID NO: 20 - protein, 12 aa, Homo sapiens
EAIYAAPFAKKK

SEQ ID NO: 21 - protein, 7 aa, Homo sapiens
LRRASLG

SEQ ID NO: 22 - protein, 39 aa, Homo sapiens
KTFCGTPEYLAPEVRREPRILSEEEQEMFRDFDYIADWC

SEQ ID NO: 23 - protein, 1130 aa, Homo sapiens [sequence omitted]

SEQ ID NO: 24 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGGVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

(19) United States
(12) Patent Application Publication
(10) Pub. No.: US 2007/0173488 A1
(43) Pub. Date: Jul. 26, 2007

(54) Pyrazolothiazole Protein Kinase Modulators

(75) Inventors: Pierre-Yves Bounaud, San Diego, CA (US); Stephanie A. Hopkins, Poway, CA (US); Elizabeth Anne Jefferson, La Jolla, CA (US); Mark E. Wilson, Ramona, CA (US); Melissa S. Wong, San Diego, CA (US); Toufike Kanouni, Del Mar, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: SGX Pharmaceuticals, Inc., San Diego, CA (US)

(21) Appl. No.: 11/560,460

(22) Filed: Nov. 16, 2006

Related U.S. Application Data
(60) Provisional application No. 60/737,702, filed on Nov. 16, 2005.

(51) Int. Cl. A61K 31/55; A61K 31/454; A61K 31/43; C07D 487/02

(52) U.S. Cl. 514/210.21; 514/366; 548/153; 514/217.08; 514/321; 540/603; 546/199

(57) ABSTRACT

The present invention provides pyrazolothiazole kinase modulators, methods of treating certain disease states, such as cancer, and pharmaceutical composition thereof.

### CROSS-REFERENCE TO RELATED APPLICATIONS

This application claims the benefit of U.S. Provisional Patent Application Ser. No. 60/737,702 entitled “Pyrazolothiazole Protein Kinase Modulators”, filed Nov. 16, 2005. Priority of the filing date is hereby claimed, and the disclosure of the application is hereby incorporated by reference.

### BACKGROUND OF THE INVENTION

Mammalian protein kinases are important regulators of cellular functions. Because disfunctions in protein kinase activity have been associated with several diseases and disorders, protein kinases are targets for drug development.

The tyrosine kinase receptor, FMS-like tyrosine kinase 3 (FLT3), is implicated in cancers, including leukemia, such as acute myeloid leukemia (AML), acute lymphoblastic leukemia (ALL), and myelodysplasia. About one-quarter to one-third of AML patients have FLT3 mutations that lead to constitutive activation of the kinase and downstream signaling pathways. Although in normal humans, FLT3 is expressed mainly by normal myeloid and lymphoid progenitor cells, FLT3 is expressed in the leukemic cells of 70-80% of patients with AML and ALL. Inhibitors that target FLT3 have been reported to be toxic to leukemic cells expressing mutated and/or constitutively-active FLT3. Thus, there is a need to develop potent FLT3 inhibitors that may be used to treat diseases and disorders such as leukemia.

The Abelson non-receptor tyrosine kinase (c-Abl) is involved in signal transduction, via phosphorylation of its substrate proteins. In the cell, c-Abl shuttles between the cytoplasm and nucleus, and its activity is normally tightly regulated through a number of diverse mechanisms. Abl has been implicated in the control of growth-factor and integrin signaling, cell cycle, cell differentiation and neurogenesis, apoptosis, cell adhesion, cytoskeletal structure, and response to DNA damage and oxidative stress.

The c-Abl protein contains approximately 1150 amino-acid residues, organized into a N-terminal cap region, an SH3 and an SH2 domain, a tyrosine kinase domain, a nuclear localization sequence, a DNA-binding domain, and an actin-binding domain.

Chronic myelogenous leukemia (CML) is associated with the Philadelphia chromosomal translocation, between chromosomes 9 and 22. This translocation generates an aberrant fusion between the bcr gene and the gene encoding c-Abl. The resultant Bcr-Abl fusion protein has constitutively active tyrosine-kinase activity. The elevated kinase activity is reported to be the primary causative factor of CML, and is responsible for cellular transformation, loss of growth-factor dependence, and cell proliferation.

The 2-phenylaminopyrimidine compound imatinib (also referred to as STI-571, CGP 57148, or Gleevec) has been identified as a specific and potent inhibitor of Bcr-Abl, as well as two other tyrosine kinases, c-kit and platelet-derived growth factor receptor. Imatinib blocks the tyrosine-kinase activity of these proteins. Imatinib has been reported to be an effective therapeutic agent for the treatment of all stages of CML. However, the majority of patients with advanced-stage or blast crisis CML suffer a relapse despite continued imatinib therapy, due to the development of resistance to the drug. Frequently, the molecular basis for this resistance is the emergence of imatinib-resistant variants of the kinase domain of Bcr-Abl. The most commonly observed underlying amino-acid substitutions include Glu255Lys, Thr315Ile, Tyr293Phe, and Met351Thr.

MET was first identified as a transforming DNA rearrangement (TPR-MET) in a human osteosarcoma cell line that had been treated with N-methyl-N′-nitro-nitrosoguanidine (Cooper et al. 1984). The MET receptor tyrosine kinase (also known as hepatocyte growth factor receptor, HGFR, MET or c-Met) and its ligand hepatocyte growth factor (“HGF”) have numerous biological activities including the stimulation of proliferation, survival, differentiation and morphogenesis, branching tubulogenesis, cell motility and invasive growth. Pathologically, MET has been implicated in the growth, invasion and metastasis of many different forms of cancer including kidney cancer, lung cancer, ovarian cancer, liver cancer and breast cancer. Somatic, activating mutations in MET have been found in human carcinoma metastases and in sporadic cancers such as papillary renal cell carcinoma. The evidence is growing that MET is one of the long-sought oncogenes controlling progression to metastasis and therefore a very interesting target. In addition to cancer there is evidence that MET inhibition may have value in the treatment of various indications including: Listeria invasion, Osteolysis associated with multiple myeloma, Malaria infection, diabetic retinopathies, psoriasis, and arthritis.

The tyrosine kinase RON is the receptor for the macrophage stimulating protein and belongs to the MET family of receptor tyrosine kinases. Like MET, RON is implicated in growth, invasion and metastasis of several different forms of cancer including gastric cancer and bladder cancer.

The cyclin dependent kinases (“CDKs”) are serine/threonine kinases responsible for control of the cell cycle. The mammalian cell cycle comprises a programmed sequence of events begining with the first growth or gap (G1) phase followed by the DNA synthesis (S) phase, to replicate the chromosomes, another growth or gap phase (G2) and finally mitosis (M phase) and cell division. It is the transition between the cell cycle phases that is controlled by the CDKs. CDKs are activated by interaction with cyclins, regulatory proteins which are expressed in an oscillating fashion in phase with the cell cycle. For example, the D-type cyclins activate CDK4 and CDK6 to control entry into S phase (G1-S transition). Cyclin A pairs with CDK2 to regulate the S-G2 transition and CDK1/cyclin B promotes the G2-M transition. The critical importance of cell cycle control in tumor growth suggests that CDK inhibition will prove a useful strategy for cancer therapy. This view is supported by substantial evidence including the upregulation of cyclins (especially cyclin D) in human tumors, the activation of CDKs by mutation in the kinase itself (e.g. CDK4) or in regulators (e.g. the gene for INK4) and the effect of CDK inhibiton on tumor growth in animal models. CDK1, CDK2, CDK4 and CDK6 are the most thoroughly studied CDKs although several other CDKs likely also play important roles in human disease.

Aurora kinases, particularly Aurora-A (“AurA”) and Aurora-B (“AurB”), have attracted considerable interest as targets for cancer therapeutics. They are involved in the regulation of mitosis and inhibitors of Aurora kinases have been shown to effectively suppress the growth of tumors in animal models.

3-Phosphoinositide-dependent kinase 1 (“PDK1”) is a Ser/Thr protein kinase that can phosphorylate and activate a number of kinases in the AGC kinase super family, including Akt/PKB, protein kinase C (PKC), PKC-related kinases (PRK1 and PRK2), p70 ribobsomal S6-kinase (S6K1), and serum and glucocorticoid-regulated kinase (SGK). The first identified PDK1 substrate is the proto-oncogene Akt. Numerous studies have found a high level of activated Akt in a large percentage (30-60%) of common tumor types, including melanoma and breast, lung, gastric, prostate, hematological and ovarian cancers. The PDK1/Akt signaling pathway thus represents an attractive target for the development of small molecule inhibitors that may be useful in the treatment of cancer. Feldman et al., JBC Papers in Press. Published on Mar. 16, 2005 as Manuscript M501367200.

Kinase inhibitors that target more than one kinase implicated in cancer have several advantages over inhibitors specific for individual kinase targets. This is especially true when the targeted kinases have distinct roles in tumorigenesis. For example, a specific inhibitor of a small array of targets such Aurora kinases, KDR (VEGFR2) and MET could simultaneously disrupt cell division, angiogenesis and metastasis through these three targets.

Because kinases have been implicated in numerous diseases and conditions, such as cancer, there is a need to develop new and potent protein kinase inhibitors that can be used for treatment. The present invention fulfills these and other needs in the art. Although certain protein kinases are specifically named herein, the present invention is not limited to inhibitors of these kinases, and, includes, within its scope, inhibitors of related protein kinases, and inhibitors of homologous proteins.

### BRIEF SUMMARY OF THE INVENTION

In one aspect, the present invention provides a pyrazolothiazole kinase modulator having the formula:

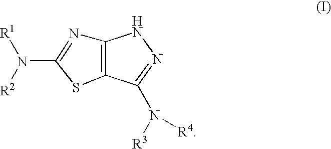

In Formula (I), R¹ and R³ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² and R⁴ are independently —C(X¹)R⁵, —SO₂R⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. X¹ is independently ═N(R⁷), ═S, or ═O, wherein R⁷ is hydrogen, cyano, —NR⁸R⁹, substituted or unsubstituted alkyl, substituted or unsubstituted heteroalkyl, substituted or unsubstituted aryl, or substituted or unsubstituted heteroaryl.

R⁵ is independently —NR⁸R⁹, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is independently —NR⁸R⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁰ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ and R², R³ and R⁴, and R⁸ and R⁹ are, independently, optionally joined with the nitrogen to which they are attached to form substituted or unsubstituted heterocycloalkyl, or substituted or unsubstituted heteroaryl.

In another aspect, the present inventions provides a method of modulating the activity of a protein kinase. The method includes contacting the protein kinase with a pyrazolothiazole compound of the present invention.

In another aspect, the present invention provides a method of modulating the activity of a protein kinase (e.g. a receptor tyrosine kinase, or a kinase selected from Abelson tyrosine kinase, Ron receptor tyrosine kinase, Met receptor tyrosine kinase, 3-Phosphoinositide-dependent kinase 1, Aurora kinases, Cyclin-dependent kinases, nerve growth factor receptor (TRKC), Colony stimulating factor 1 receptor (CSF1R), and vascular endothelial growth factor receptor 2 (VEGFR2, KDR)). The method includes contacting the protein tyrosine kinase with a pyrazolothiazole compound of the present invention.

In another aspect, the present invention provides a pharmaceutical composition including a pyrazolothiazole compound of the present invention in admixture with a pharmaceutically acceptable excipient.

### BRIEF DESCRIPTION OF THE DRAWINGS

FIG. 1 shows the wild-type ABL numbering according to ABL exon Ia.

FIG. 2 shows the Homo sapiens MET full-length sequence.

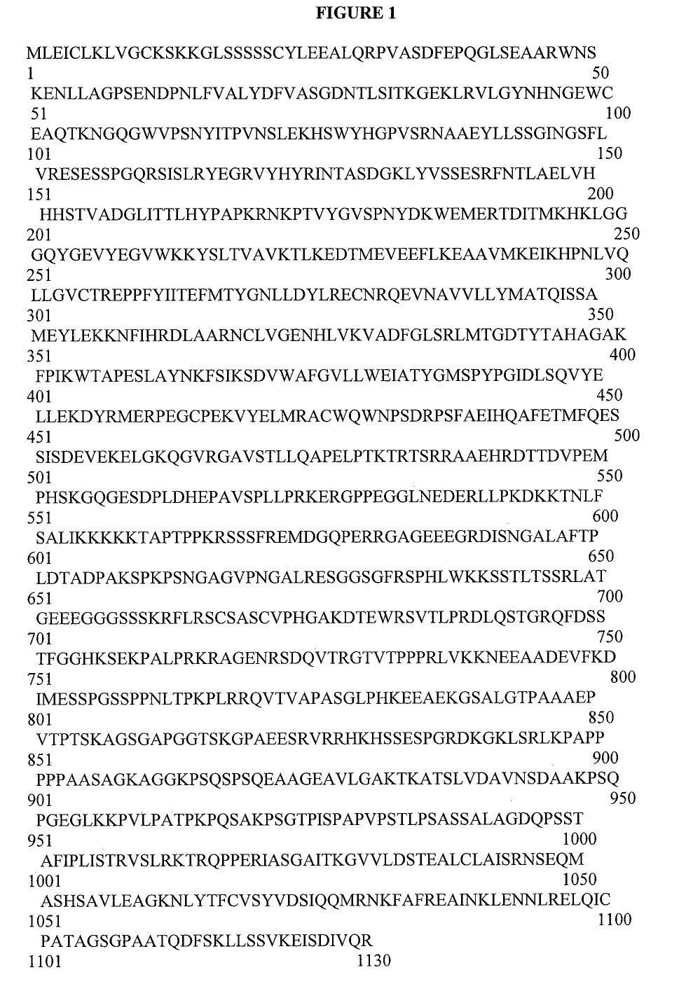

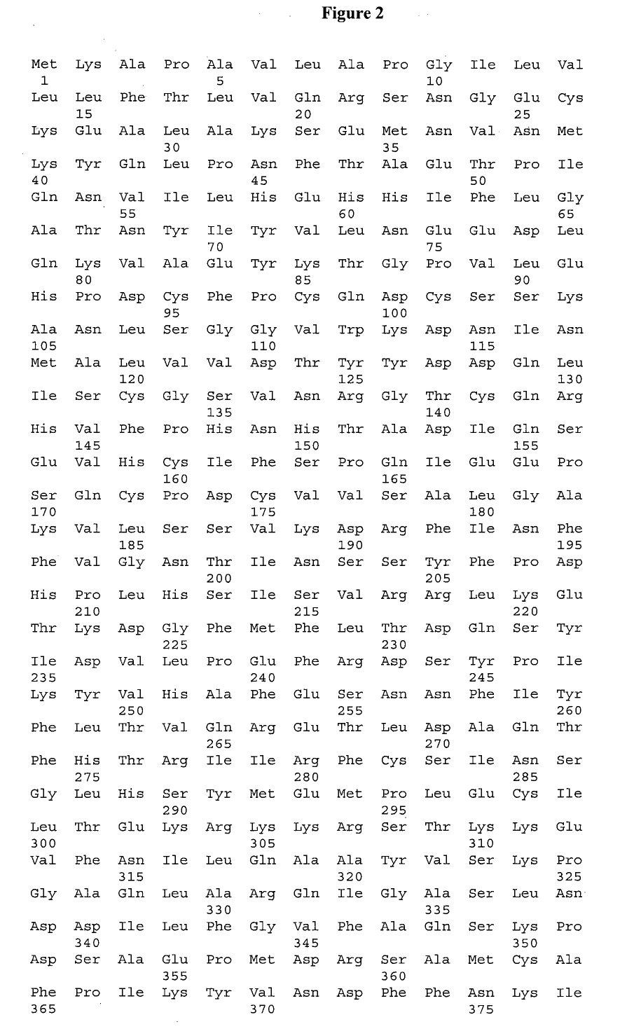

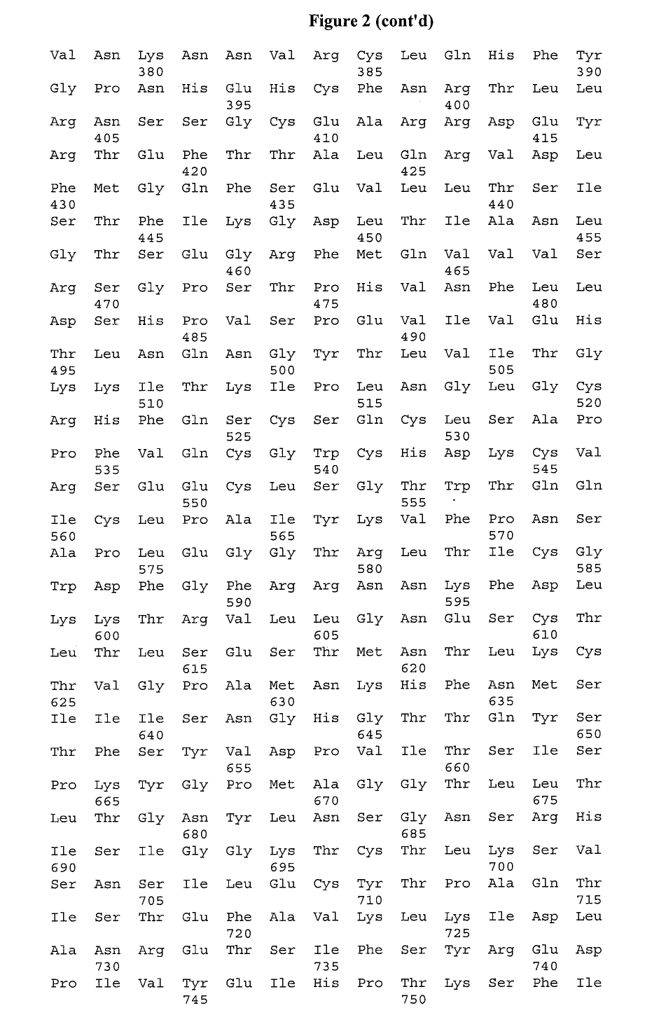

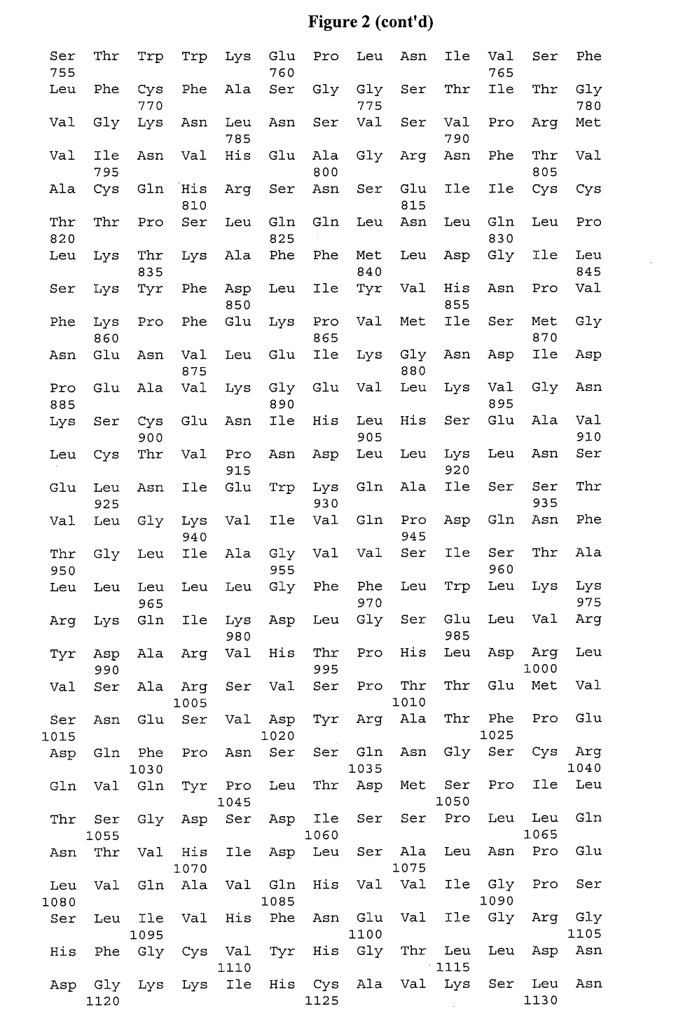

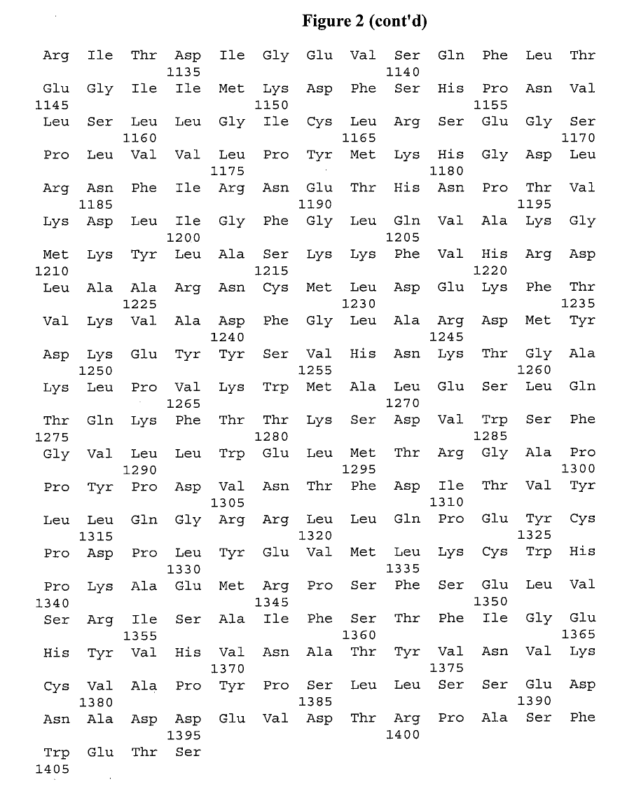

### DETAILED DESCRIPTION OF THE INVENTION

Definitions

Abbreviations used herein have their conventional meaning within the chemical and biological arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e. unbranched) or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. As described above, heteroalkyl groups, as used herein, include those groups that are attached to the remainder of the molecule through a heteroatom, such as —C(O)R′, —C(O)NR′, —NR′R, —OR′, —SR, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

An “alkylesteryl,” as used herein, refers to a moiety having the formula R′—C(O)O—R″, wherein R′ is an alkylene moiety and R″ is an alkyl moiety.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

The term “cycloalkylalkyl” refers to a 3 to 7 membered cycloalkyl group attached to the remainder of the molecule via an unsubstituted alkylene group. Recitation of a specific number of carbon atoms (e.g. C₁-C₁₀ cycloalkylalkyl) refers to the number of carbon atoms in the alkylene group.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo (C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized (e.g. pyridine N-oxide), and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent derivatives of aryl and heteroaryl, respectively.

For brevity, the term “aryl” when used in combination with other terms (e.g., aryloxo, arylthioxo, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

The term “oxo” as used herein means an oxygen that is double bonded to a carbon atom.

Each of above terms (e.g., “alkyl,” “heteroalkyl,” “cycloalkyl, and “heterocycloalkyl”, “aryl,” “heteroaryl” as well as their divalent radical derivatives) are meant to include both substituted and unsubstituted forms of the indicated radical. Preferred substituents for each type of radical are provided below.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R′R″″, —OC(O)R′, —C(O)R′, —CO₂R′,—C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From above discussion of substituents, one of skill in art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl radicals above, exemplary substituents for aryl and heteroaryl groups ( as well as their divalent derivatives) are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″R′″ and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

The compounds of the present invention may exist as salts. The present invention includes such salts. Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfaric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the enantiomers, racemates, diastereomers, tautomers, geometric isomers, stereoisometric forms that may be defined, in terms of absolute stereochemistry, as (R)—or (S)— or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)— and (S)—, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

The term “pharmaceutically acceptable salts” is meant to include salts of active compounds which are prepared with relatively nontoxic acids or bases, depending on the particular substituent moieties found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, physiological conditions.

The terms “treating” or “treatment” in reference to a particular disease includes prevention of the disease.

Pyrazolothiazole Kinase Modulators

In one aspect, the present invention provides a pyrazolothiazole kinase modulator having the formula:

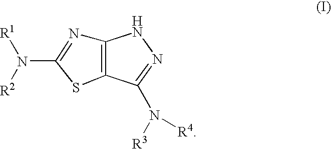

In Formula (I), R¹, R², R³, and R⁴ are as defined above.

In some embodiments, R¹ and R³ are independently hydrogen, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. In some embodiments, R² and R⁴ are independently —C(X¹)R⁵, —SO₂R⁶, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. In some embodiments, X¹ is independently ═N(R⁷), ═S, or ═O, wherein R⁷ is hydrogen, cyano, —NR⁸R⁹, R¹¹-substituted or unsubstituted alkyl, R¹¹-substituted or unsubstituted heteroalkyl, R¹¹-substituted or unsubstituted aryl, or R¹¹-substituted or unsubstituted heteroaryl;

In some embodiments, R⁵ is independently —NR⁸R⁹, —OR¹⁰, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. In some embodiments, R⁶ is independently —NR⁸R⁹, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. In some embodiments, R⁸ and R⁹ are independently hydrogen, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl.

In some embodiments, R¹⁰ is independently R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl.

In some embodiments, R¹ and R², R³ and R⁴, and R⁸ and R⁹ are, independently, optionally joined with the nitrogen to which they are attached to form R¹¹-substituted or unsubstituted heterocycloalkyl, or R¹¹-substituted or unsubstituted heteroaryl.

R¹¹ is independently halogen; -L¹-C(X²)R¹²; -L¹-OR¹³; -L¹-NR¹⁴R¹⁵;-L¹-S(O)ₘR¹⁶; —CN; —NO₂; —CF₃; (1) unsubstituted C₃-C₇ cycloalkyl; (2) unsubstituted 3 to 7 membered heterocycloalkyl; (3) unsubstituted heteroaryl; (4) unsubstituted aryl; (5) substituted C₃-C₇ cycloalkyl; (6) substituted 3 to 7 membered heterocycloalkyl; (7) substituted aryl; (8) substituted heteroaryl; (9) unsubstituted C₁-C₂₀ alkyl; (10) unsubstituted 2 to 20 membered heteroalkyl; (11) substituted C₁-C₂₀ alkyl; or (12) substituted 2 to 20 membered heteroalkyl.

Substituents (5), (6), (11), and (12) are independently substituted with an oxo, —OH, —CF₃, —COOH, cyano, halogen, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, R¹⁸-substituted or unsubstituted heteroaryl, -L¹-C(X²)R¹², -L¹-OR¹³, -L¹-NR¹⁴R¹⁵, or -L¹-S(O)ₘR¹⁶. Substituents (7) and (8) are independently substituted with an —OH, —CF₃, —COOH, cyano, halogen, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, R¹⁸-substituted or unsubstituted heteroaryl, -L¹-C(X²)R¹², -L¹-OR¹³, -L¹-NR¹⁴R¹⁵, or -L¹-S(O)ₘR¹⁶.

X² is independently ═S, ═O, or ═NR²⁷. R²⁷ is H, —CN, —NR⁸R⁹, —OR²⁸, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, or R¹⁸-substituted or unsubstituted heteroaryl. R²⁸ is hydrogen or R¹⁷-substituted or unsubstituted C₁-C₁₀ alkyl. The symbol m independently represents an integer from 0 to 2.

R¹² is independently hydrogen, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, R¹⁸-substituted or unsubstituted heteroaryl, —OR¹⁹, or —NR²⁰R²¹. R¹⁹, R²⁰, and R²¹ are independently hydrogen, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, or R¹⁸-substituted or unsubstituted heteroaryl. R²⁰ is optionally —S(O)₂R³⁰, or —C(O)R³⁰. R²⁰ and R²¹ are optionally joined with the nitrogen to which they are attached to form an R¹⁷-substituted or unsubstituted 3 to 7 membered heterocycloalkyl, or R¹⁸-substituted or unsubstituted heteroaryl. R³⁰ is R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, or R¹⁸-substituted or unsubstituted heteroaryl.

R¹³, R¹⁴ and R¹⁵ are independently hydrogen, -CF₃, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, R¹⁸-substituted or unsubstituted heteroaryl, —C(X³)R²², or —S(O)₂R²². R¹⁴ and R¹⁵ are optionally joined with the nitrogen to which they are attached to form an R¹⁷-substituted or unsubstituted 3 to 7 membered heterocycloalkyl, or R¹⁸-substituted or unsubstituted heteroaryl. X³ is independently ═S, ═O, or ═NR²³. R²³ is cyano, —NR⁸R⁹, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, or R¹⁸-substituted or unsubstituted heteroaryl. R²² is independently R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, R¹⁸-substituted or unsubstituted heteroaryl, or —NR²⁴R²⁵. In some embodiments, where R¹¹ is -L¹-NR¹⁴R¹⁵ and R¹⁴ or R¹⁵ is —C(X³)R²², then R²² is optionally hydrogen. R²⁴ and R²⁵ are independently hydrogen, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, or R¹⁸-substituted or unsubstituted heteroaryl. R²⁴ and R²⁵ may be joined with the nitrogen to which they are attached to form an R¹⁷-substituted or unsubstituted 3 to 7 membered heterocycloalkyl, or R¹⁸-substituted or unsubstituted heteroaryl.

R¹⁶ is independently R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, R¹⁸-substituted or unsubstituted heteroaryl, or —NR²⁶R²⁷. In some embodiments, where m is 0, R¹⁶ is optionally hydrogen. R²⁶ and R²⁷ are independently hydrogen, cyano, —NR⁸R⁹, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membere 21-substituted or unsubstituted heteroaryl. R²⁶ and R²⁷ may be joined with the nitrogen to which they are attached to form an R¹⁷-substituted or unsubstituted 3 to 7 membered heterocycloalkyl, or R¹⁸-substituted or unsubstituted heteroaryl. R²⁶ may additionally be —C(O)R³⁰.

L¹ is independently a bond, unsubstituted C₁-C₁₀ alkylene, or unsubstituted heteroalkylene. R¹⁷ is independently oxo, —OH, —COOH, —CF₃, —OCF₃, —CN, amino, halogen, R²⁸-substituted or unsubstituted 2 to 10 membered alkyl, R²⁸-substituted or unsubstituted 2 to 10 membered heteroalkyl, R²⁸-substituted or unsubstituted C₃-C₇ cycloalkyl, R²⁸-substituted or unsubstituted 3 to 7 membered heterocycloalkyl, R²⁹-substituted or unsubstituted aryl, or R²⁹-substituted or unsubstituted heteroaryl. R18 is independently —OH, —COOH, amino, halogen, —CF₃, —OCF₃, —CN, R²⁸-substituted or unsubstituted 2 to 10 membered alkyl, R²⁸-substituted or unsubstituted 2 to 10 membered heteroalkyl, R²⁸-substituted or unsubstituted C₃-C₇ cycloalkyl, R²⁸-substituted or unsubstituted 3 to 7 membered heterocycloalkyl, R²⁹-substituted or unsubstituted aryl, or R²⁹-substituted or unsubstituted heteroaryl. R²⁸ is independently oxo, —OH, —COOH, amino, halogen, —CF₃, —OCF₃, —CN, unsubstituted C₁-C₁₀ alkyl, unsubstituted 2 to 10 membered heteroalkyl, unsubstituted C₃-C₇ cycloalkyl, unsubstituted 3 to 7 membered heterocycloalkyl, unsubstituted aryl, unsubstituted heteroaryl. R²⁹ is independently —OH, —COOH, amino, halogen, —CF₃, —OCF₃, —CN, unsubstituted C₁-C₁₀ alkyl, unsubstituted 2 to 10 membered heteroalkyl, unsubstituted C₃-C₇ cycloalkyl, unsubstituted 3 to 7 membered heterocycloalkyl, unsubstituted aryl, unsubstituted heteroaryl.

In some embodiments, R¹ is hydrogen. In some embodiments, R³ is hydrogen. In some embodiments, R² is —C(X¹)R⁵, R¹¹-substituted or unsubstituted alkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl, wherein X¹ is ═O.

In some embodiments, R² is —C(X¹)R⁵. In some embodiments, R⁵ is R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. In some embodiments, R⁵ is R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. In some embodiments, R⁵ is R¹¹-substituted or unsubstituted cycloalkyl.

In some embodiments, R⁴ is selected from —C(X¹)R⁵, R¹¹-substituted or unsubstituted alkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl, wherein X¹ is ═O. In some embodiments, R⁴ is R¹¹-substituted or unsubstituted alkyl, wherein R¹¹ is (1), (2), (3), (4), (5), (6), (7), or (8). In some embodiments, R⁴ is selected from —C(X¹)R⁵, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl, wherein X¹ is ═O. In some embodiments, R⁴ is —C(X¹)R⁵. In some embodiments, the R⁵ that forms part of R⁴ is R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. In some embodiments, the R⁵ within the R⁴ is R¹¹-substituted or unsubstituted heteroaryl, or R¹¹-substituted or unsubstituted aryl. In some embodiments, the R¹¹ that forms part of the R⁵ within R⁴ is halogen, -L¹-S(O)ₘR¹⁶, -L¹-OR¹³, -L¹-C(X²)R¹², -L¹-NR¹⁴R¹⁵, (3), (4), (7), or (8). In some embodiments, the L¹ of R¹¹ within R⁴ is a bond, or methylene. In some embodiments, m is 2.

In some embodiments, the R¹¹-substituted heteroaryl of R⁴, and the R¹¹-substituted aryl of R⁴ are substituted at the ortho position.

In some embodiments, R⁴ and R³ are joined with the nitrogen to which they are attached to form an R¹¹-substituted or unsubstituted 5-membered heteroaryl. In some embodiments, the R⁴ and R³ are joined with the nitrogen to which they are attached to form an R¹¹-substituted or unsubstituted heteroaryl selected from the groups consisting of R¹¹-substituted or unsubstituted pyrrolyl, R¹¹-substituted or unsubstituted imidazolyl, R¹¹-substituted or unsubstituted pyrazolyl, and R¹¹-substituted or unsubstituted triazolyl. In some embodiments, R⁴ and R³ are joined with the nitrogen to which they are attached to form an R¹¹-substituted or unsubstituted [1,2,3] triazolyl, R¹¹-substituted or unsubstituted [1,2,4] triazolyl, or R¹¹-substituted or unsubstituted [1,3,4] triazolyl. In some embodiments, the R¹¹ of the R¹¹-substituted or unsubstituted heteroaryl formed by R³ and R⁴ is halogen, -L¹-S(O)ₘR¹⁶, -L¹-OR¹³, -L¹-C(X²)R¹², -L¹-NR¹⁴K¹⁵, (3), (4), (7), or (8). In some embodiments, the R¹¹ of the R¹¹-substituted or unsubstituted heteroaryl formed by R³ and R⁴ is (7) or (8). In some embodiments, (7) and (8) are independently substituted with halogen, -L¹-OR¹³, -L¹-NR¹⁴R¹⁵, -L¹-C(X²)R¹², -L¹-S(O)ₘR¹⁶, R¹⁷-substituted or unsubstituted C₁-C₁₀ alkyl, or R¹⁸-substituted or unsubstituted heteroaryl. In some embodiments, L¹ is a bond or methylene. In some embodiments, the R¹¹-substituted heteroaryl formed by R⁴ and R³ is substituted at the ortho position.

Exemplary Syntheses

The compounds of the invention are synthesized by an appropriate combination of generally well known synthetic methods. Techniques useful in synthesizing the compounds of the invention are both readily apparent and accessible to those of skill in the relevant art, including the techniques disclosed in Elnagdi, et al., J. Heterocyclic Chem., 16: 61-64 (1979), Pawar, et al., Indian J. Chem., 28B: 866-867 (1989), Chande, et al., Indian J. Chem., 35B: 373-376 (1996), and in the following patents DE2429195 (1974), U.S. Pat. No. 6,566,363 (2003), WO05068473A1 (2005), WO05095420A1 (2005), which are incorporated in reference in their entirety for all purposes. The discussion below is offered to illustrate certain of the diverse methods available for use in assembling the compounds of the invention. However, the discussion is not intended to define the scope of reactions or reaction sequences that are useful in preparing the compounds of the present invention. The compounds of this invention may be made by the procedures and techniques disclosed in the Examples section below, as well as by known organic synthesis techniques.

In the exemplary syntheses below, the symbols R¹, R², R³, and R⁴ are, unless specified otherwise, defined as above in the section entitled “Pyrazolothiazole Kinase Modulators.”

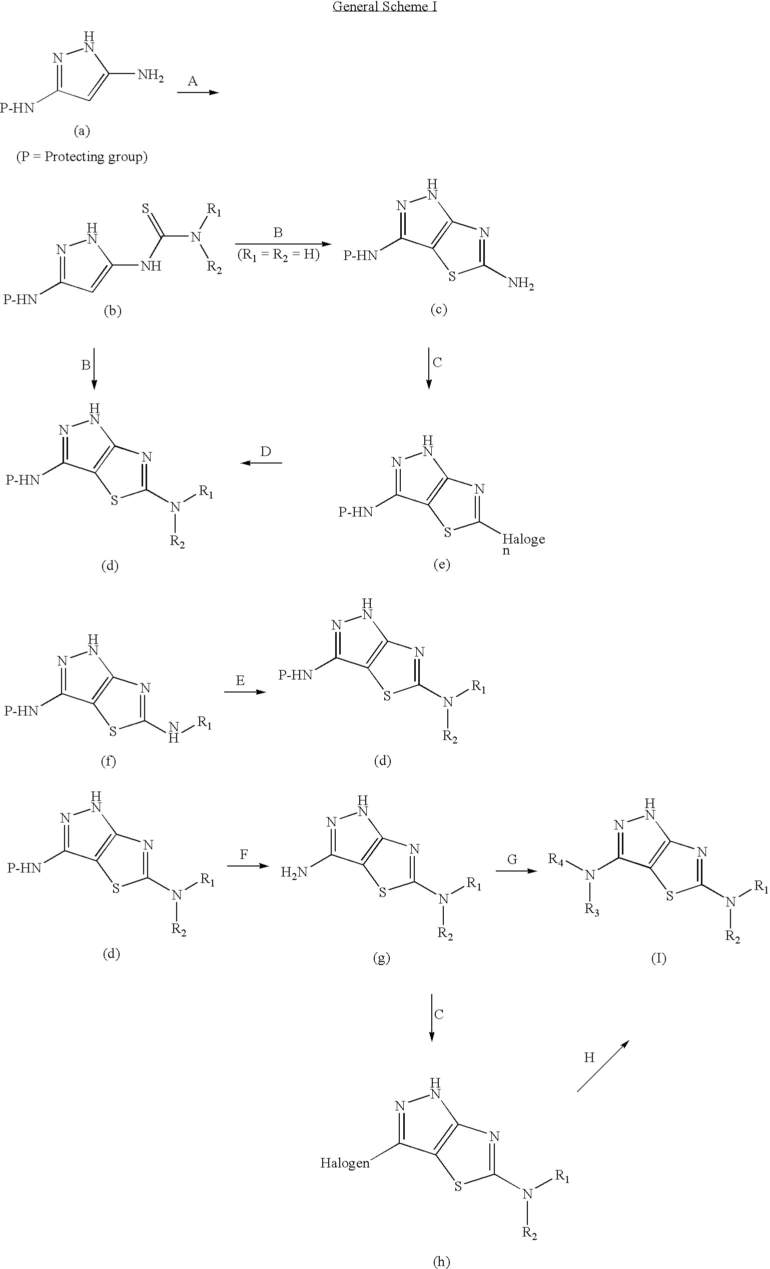

In step A of General Scheme I, synthesis of the thiourea (b) is performed by reacting a suitably protected pyrazole (a) with thiocarbonyl reagents, such as but not limited to, thiophosgene or thiocarbonyldiimidazole, followed by treatment with an amine, such as but not limited to, ammonia, ammonium hydroxide, aniline, heteroarylamine, primary or secondary amine, or alternatively pyrazole (a) is reacted with an isothiocyanate reagent, in suitable solvents such as halogenated hydrocarbons, ethereal solvents, THF, DMF, and water mixtures thereof, at temperatures ranging from −30° C. to 100° C.

In step B, synthesis of the bicyclic intermediates (c) or (d) is accomplished by reacting a derivative (b), with a suitable halogenating reagent, such as but not limited to, chlorine, bromine, iodine, ICl, N-chlorosuccinimide, N-bromosuccinimide, N-iodosuccinimide, or benzyltrimethylammonium tribromide, in suitable solvents such as acetic acid, DMF, ethereal solvents, or halogenated hydrocarbons, at temperatures ranging from −10° C. to 100° C.

In step C, synthesis of the halogenated bicyclic intermediates (e) or (h) is accomplished by reacting derivative (c), or (g) respectively, with a suitable “nitrite” reagent, such as but not limited to, sodium nitrite in acidic media or isoamyl nitrite, in the presence of the copper salt of the desired halogen, in a suitable solvent such as alcohols, ethereal solvents, DMF, or water or mixture thereof, at temperatures ranging from −78° C. to 100° C.

In step D, synthesis of the intermediate (d) is achieved by reacting halogenated intermediate (e) with a primary or secondary amine, an aniline, or a heteroarylamine in the presence or absence of a Lewis acid, in a suitable solvent such as alcohols, ethereal solvents, DMF, or DMSO, at temperatures ranging from −0° C. to 250° C., under conventional heating or microwave heating. Alternatively, intermediate (d) is obtained by reacting halogenated intermediate (e) with a primary or secondary amine, an aniline, or a heteroarylamine in the presence of a metal catalyst, such as palladium, copper, or nickel, and its appropriate ligand, such as electron-rich phosphines, N-heterocyclic carbenes, or aminophosphines, in the presence of a base, such as potassium phosphate, sodium tert-butoxide, or cesium carbonate, in a suitable solvent such as toluene, halogenated hydrocarbons, ethereal solvents, DMF, or water or mixture thereof, at temperatures ranging from 0° C. to 180° C., as exemplified in Hartwig et al. J. Org. Chem. 2003, 68, 2861-73.

Step E exemplifies another synthesis of intermediate (d). Treatment of intermediate (f), optionally protected at the NH site, with suitable electrophiles such as carboxylic acids (in combination with amide coupling reagents such as but not limited to DCC, EDC, HATU, HBTU, PyBOP), acid chlorides, isocyanates, isothiocyanates, sulfonyl chlorides, imidoyl chlorides, imidoate esters or isothioureas, in the presence of absence of base such as but not limited to triethylamine, diisopropylethylamine, sodium bicarbonate, or sodium carbonate, in suitable solvents such as ethereal solvents, DMF, DMSO, at temperatures ranging from 20° C. to 200° C., followed by basic hydrolysis with bases such as but not limited to sodium hydroxide or primary alkyl amines, in suitable solvents such as alcohols, ethereal solvents, DMF, and water mixtures thereof, at temperatures ranging from 0° C. to 100° C. successfully generates intermediate (d). Alternatively, intermediate (f), optionally protected at the NH site, can be treated with an aldehyde in the presence of a reducing agent such as but not limited to sodium borohydride or sodium cyanoborohydride to give intermediate (d).

In step F, bicyclic intermediate (d) is subjected to standard deprotecting conditions to give intermediate (g). Such conditions are well known to a person skilled in the art and exemplified in Greene, et al., Protective Groups in Organic Synthesis, 3rd ed. John Wiley & Sons (1999).

Step G shows the exemplary synthesis of end product of general formula (I). The treatment of intermediate (g), optionally protected at the NH site, with suitable electrophiles such as carboxylic acids (in combination with amide coupling reagents such as but not limited to DCC, EDC, HATU, HBTU, PyBOP), acid chlorides, isocyanates, isothiocyanates, sulfonyl chlorides, imidoyl chlorides, imidoate esters or isothioureas, in suitable solvents such as ethereal solvents, DMF, DMSO, at temperatures ranging from 20° C. to 200° C., followed by basic hydrolysis with bases such as but not limited to sodium hydroxide or primary alkyl amines, in suitable solvents such as alcohols, ethereal solvents, DMF, and water mixtures thereof, at temperatures ranging from 0° C. to 100° C. affords the desired product (I). Alternatively, reaction of intermediate (g) with aldehydes in the presence of a reducing agent, such as but not limited to sodium borohydride or sodium cyanoborohydride, in suitable solvents such as alcohols, ethereal solvents, halogenated hydrocarbons, or DMF, at temperatures ranging from 0° C. to 100° C. affords the desired product (I). In another example, reaction of intermediate (g) with aldehydes, in the presence or absence of dehydrating agent, in a suitable solvent such as alcohols, ethereal solvents, or toluene, at temperatures ranging from 0° C. to 100° C., forms an imine intermediate that is further treated with isocyanides in the presence of a base, such as but not limited potassium carbonate, in a suitable solvent, such as ethereal solvents or DMF, at temperatures ranging from 0° C. to 100° C. to provide the desired product (I), where R₃ and R₄ are linked to form a ring. In another example, intermediate (g) can be reacted with cyclizing reagents such as but not limited to 1,4-dicarbonyl reagents, substituted oxadiazoles, or substituted pyranones, in the presence or absence of base, neat or in a suitable solvent such as acetonitrile, toluene, ethereal solvents, or pyridine, at temperatures ranging from 0° C. to 180° C., to form desired product (I).

Step H shows yet another example of the synthesis of the end product (I). Intermediate (h), optionally protected at the NH site, is treated with a primary or secondary amine, an aniline, a heteroarylamine, or a heteroaryl group bearing an “anionic” nitrogen, such as pyrrole, imidazole, triazole, or tetrazole, in a suitable solvent, such as alcohols, ethereal solvents, DMF, or DMSO, at temperatures ranging from 0° C. to 250° C., under conventional heating or microwave heating to afford the desired product (I). Alternatively, the substitution of the halogen by various amines, such as primary or secondary amines, anilines, or heteroarylamines may be achieved in the presence of a metal catalyst, such as palladium, copper, or nickel, and its appropriate ligand, such as electron-rich phosphines, N-heterocyclic carbenes, or aminophosphines, in the presence of a base, such as but not limited to potassium phosphate, sodium tert-butoxide, or cesium carbonate, in a suitable solvent such as toluene, halogenated hydrocarbons, ethereal solvents, DMF, or water or mixture thereof, at temperatures ranging from 0° C. to 180° C., as exemplified in Hartwig et al. J. Org. Chem. 2003, 68, 2861-73.

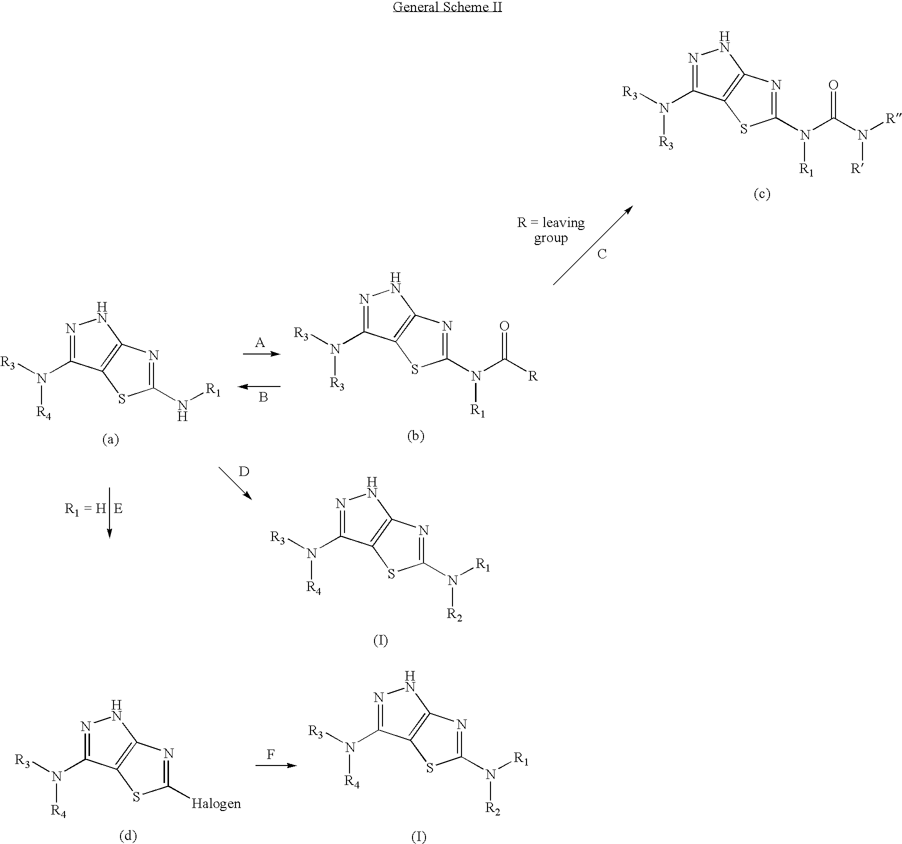

Step A of General Scheme II shows the exemplary synthesis of end product (b). The treatment of intermediate (a), optionally protected at the NH site, with suitable acylating species such as carboxylic acids (in combination with amide coupling reagents such as but not limited to DCC, EDC, HATU, HBTU, PyBOP) or acid, in suitable solvents such as ethereal solvents, DMF, DMSO, at temperatures ranging from 20° C. to 200° C., followed by basic hydrolysis with bases such as but not limited to sodium hydroxide or primary alkyl amines, in suitable solvents such as alcohols, ethereal solvents, DMF, and water mixtures thereof, at temperatures ranging from 0° C. to 100° C. affords the desired product (b).

Step B describes a method to hydrolyze an acyl or carbamate group off pyrazolothiazole (b). Treatment of (a) with a strong acid such as but not limited to hydrochloric acid, sulfuric acid, or perchloric acid in aqueous medium under thermal or microwave conditions at temperatures ranging from 50 to 200° C. provides said pyrazolothiazole (b).

Step C describes a method to prepare pyrazolothiazole ureas (c) from pyrazolothiazole carbamates (b). Treatment of (b) with an amine in a suitable solvent such as alcohols, ethereal solvents, DMF, or DMSO, under thermal or microwave conditions at temperatures ranging from 50 to 200° C. affords end product (c).

Step D shows an exemplary synthesis of end product (I). Reaction of pyrazolothiazole (a) with an activated aryl or heteroaryl halide in presence of a base in a suitable solvent such as DMSO, NMP, or DMF at temperatures ranging from 0 to 150° C. affords end product (I). Alternatively, the substitution at the amine group with aryl or heteroaryl halides may be achieved in the presence of a metal catalyst, such as palladium, copper, or nickel, and its appropriate ligand, such as electron-rich phosphines, N-heterocyclic carbenes, or aminophosphines, in the presence of a base, such as but not limited to potassium phosphate, sodium tert-butoxide, or cesium carbonate, in a suitable solvent such as toluene, halogenated hydrocarbons, ethereal solvents, DMF, or water or mixture thereof, at temperatures ranging from 0° C. to 180° C., as exemplified in Hartwig et al. J. Org. Chem. 2003, 68, 2861-73.

In step E, synthesis of the halogenated intermediate (d) is accomplished by reacting derivative (a) with a suitable “nitrite” reagent, such as but not limited to, sodium nitrite in acidic media or isoamyl nitrite, in the presence of the copper salt of the desired halogen, in a suitable solvent such as alcohols, ethereal solvents, DMF, or water or mixture thereof, at temperatures ranging from −78° C. to 100° C.

In step F, synthesis of end product (I) is achieved by reacting halogenated intermediate (d) with a primary or secondary amine, an aniline, or a heteroarylamine in the presence or absence of a Lewis acid, in a suitable solvent such as alcohols, ethereal solvents, DMF, or DMSO, at temperatures ranging from −0° C. to 250° C., under conventional heating or microwave heating. Alternatively, end product (I) is obtained by reacting halogenated intermediate (d) with a primary or secondary amine, an aniline, or a heteroarylamine in the presence of a metal catalyst, such as palladium, copper, or nickel, and its appropriate ligand, such as electron-rich phosphines, N-heterocyclic carbenes, or aminophosphines, in the presence of a base, such as potassium phosphate, sodium tert-butoxide, or cesium carbonate, in a suitable solvent such as toluene, halogenated hydrocarbons, ethereal solvents, DMF, or water or mixture thereof, at temperatures ranging from 0° C. to 180° C., as exemplified in Hartwig et al. J. Org. Chem. 2003, 68, 2861-73.

The general methods illustrated above are further exemplified by the transformations presented in Schemes 1-6.

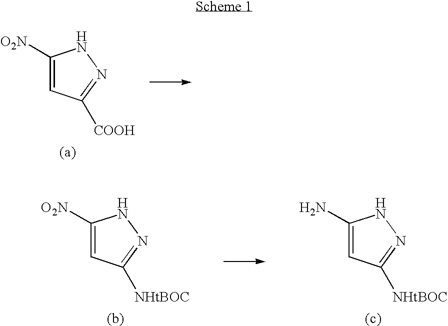

In Scheme 1, 5-nitro-2H-pyrazole-3-carboxylic acid (a) is treated with diphenylphosphorylazide in tert-butanol to afford pyrazole (b) by Curtius rearrangement. Compound (b) is further reduced to aminopyrazole (c) under hydrogen atmosphere in presence of a palladium catalyst.

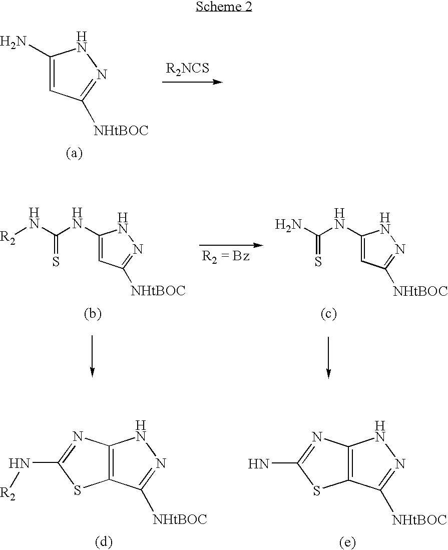

In Scheme 2, aminopyrazole (a) is treated with an isothiocyanate to generate thiourea (b). In the case of R₂=benzoyl (Bz), the benzoyl group is removed under basic conditions such as sodium hydroxide to provide thiourea (c). Both thioureas (b) and (c) are cyclized to pyrazolothiazoles (d) and (e) respectively in the presence of a bromo cation source, such as bromine in acetic acid, or N-bromosuccinimide.

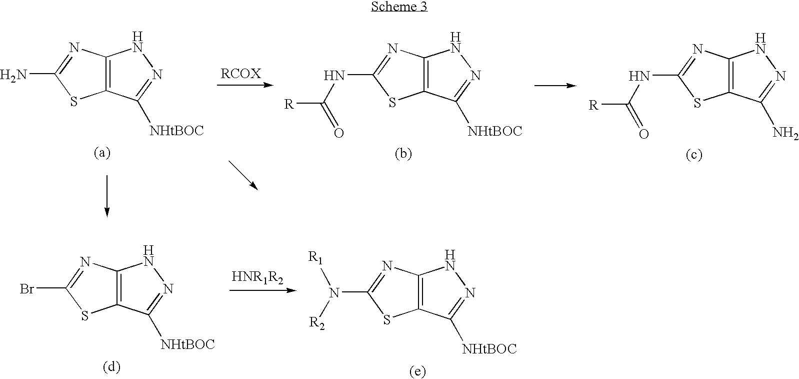

In Scheme 3, pyrazolothiazole (a) is first treated with an excess of acyl chloride or “activated” carboxylic acid under thermal conditions, followed by a scavenging step with a primary amine, to provide pyrazolothiazole (b). The BOC protecting group on compound (b) is removed by acidic treatment in the presence of a cation scavenger, such as thiophenol on polymer support, to give aminopyrazolothiazole (c). Alternatively, pyrazolothiazole (a) is treated with an alkyl nitrite, such as isoamyl nitrite or tert-butyl nitrite, in the presence of copper(I) bromide to give bromopyrazolothiazole (d). Compound (d) is then converted to pyrazolothiazole (e) in the presence of various amines. Alternatively, pyrazolothiazole (a) is treated with an aldehyde under reducing conditions, such as sodium triacetoxyborohydride, to give pyrazolothiazole (e).

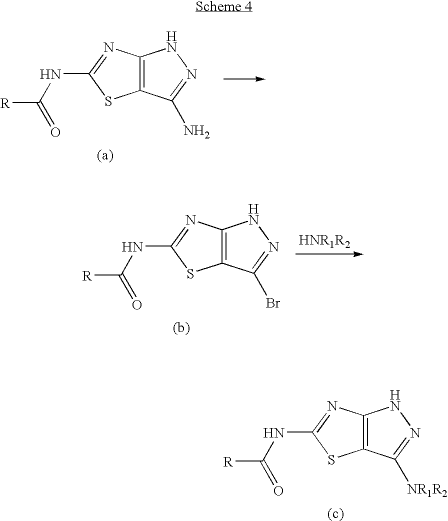

In Scheme 4, pyrazolothiazole (a) undergoes diazotization in presence of copper(II) bromide to afford bromopyrazolothiazole (b). Compound (b) is then treated with various amines, in the presence or absence of metal or Lewis acid catalyst, under thermal or microwave conditions to yield pyrazolothiazoles (c).

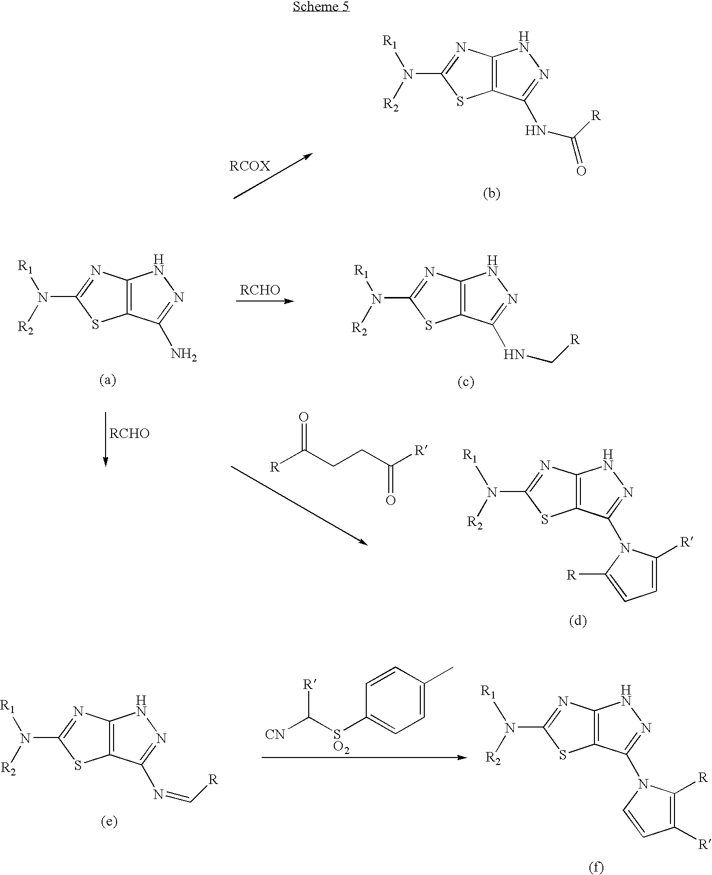

In Scheme 5, pyrazolothiazole (a) is first treated with an excess of acyl chloride or “activated” carboxylic acid under thermal conditions, followed by a scavenging step with a primary amine, to provide pyrazolothiazole (b). In another example, pyrazolothiazole (a) is treated with an aldehyde in the presence of a reducing agent such as sodium cyanoborohydride to give substituted aminopyrazolothiazole (c). Alternatively, pyrazolothiazole (a) is reacted with an aldehyde in alcoholic solvent under thermal conditions to form imine (e), which is immediately reacted with optionally substituted tosylmethyl isocyanide in the presence of a base under thermal conditions to provide imidazole f. In another example, pyrazolothiazole (a) is treated with a 1,4-dicarbonyl reagent under thermal or microwave conditions to give pyrrole (d).

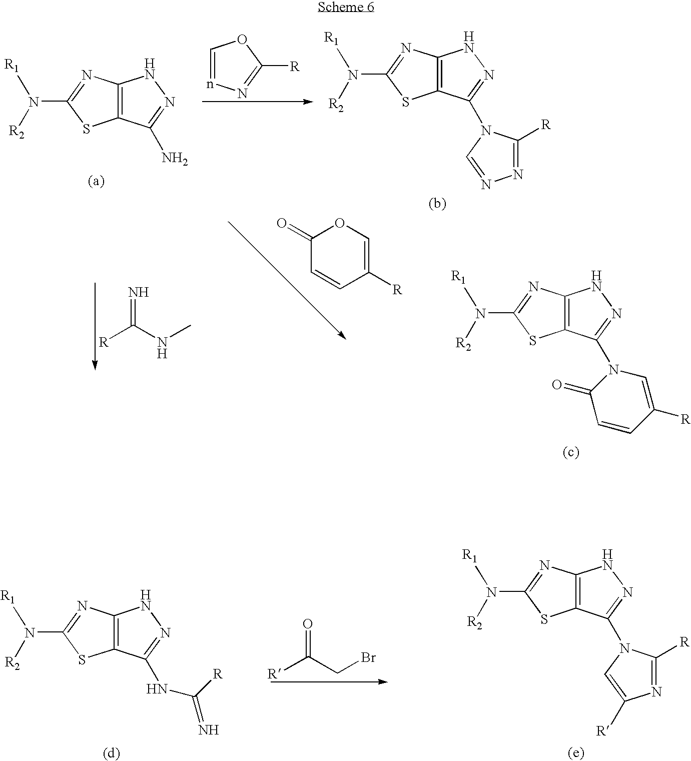

In Scheme 6, pyrazolothiazole (a) is treated with an excess of suitably substituted oxadiazole under thermal conditions to provide pyrazole (b). Alternatively, pyrazolothiazole (a) is treated with suitably substituted pyran-2-one in presence of a base to give pyrazolothiazole (c). In another example, pyrazolothiazole (a) is treated with an imidate species under thermal conditions to provide imidate (d), which is further reacted under thermal conditions with a bromoacetylketone or bromopyruvate species in presence of a base to cyclize to pyrazolothiazole (e).

The compounds of the present invention may be synthesized using one or more protecting groups generally known in the art of chemical synthesis. The term “protecting group” refers to chemical moieties that block some or all reactive moieties of a compound and prevent such moieties from participating in chemical reactions until the protective group is removed, for example, those moieties listed and described in Greene, et al., Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(0)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking or protecting groups include, for example: 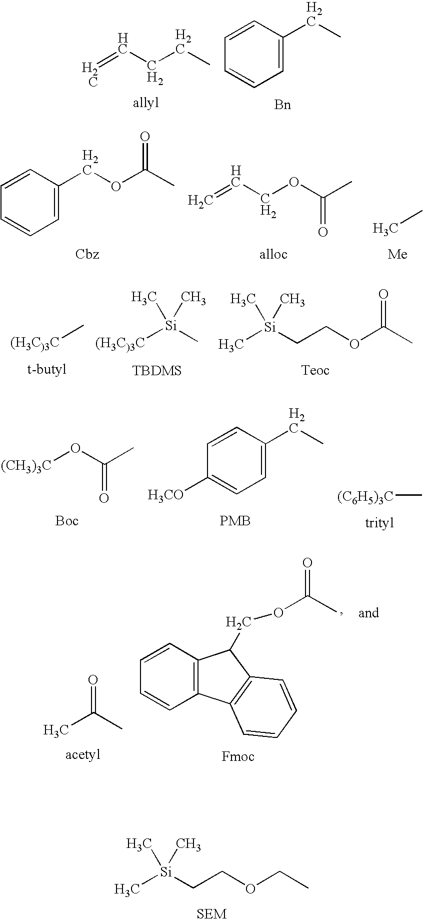
Methods of Inhibiting Kinases

In another aspect, the present invention provides methods of modulating protein kinase activity using the pyrazolothiazole kinase modulators of the present invention. The term “modulating kinase activity,” as used herein, means that the activity of the protein kinase is increased or decreased when contacted with a pyrazolothiazole kinase modulator of the present invention relative to the activity in the absence of the pyrazolothiazole kinase modulator. Therefore, the present invention provides a method of modulating protein kinase activity by contacting the protein kinase with a pyrazolothiazole kinase modulator of the present invention.

In an exemplary embodiment, the pyrazolothiazole kinase modulator inhibits kinase activity. The term “inhibit,” as used herein in reference to kinase activity, means that the kinase activity is decreased when contacted with a pyrazolothiazole kinase modulator relative to the activity in the absence of the pyrazolothiazole kinase modulator. Therefore, the present invention further provides a method of inhibiting protein kinase activity by contacting the protein kinase with a pyrazolothiazole kinase modulator of the present invention.

In certain embodiments, the protein kinase is a protein tyrosine kinase. A protein tyrosine kinase, as used herein, refers to an enzyme that catalyzes the phosphorylation of tyrosine residues in proteins with a phosphate donor (e.g. a nucleotide phosphate donor such as ATP). Protein tyrosine kinases include, for example, Abelson tyrosine kinases (“Abl”) (e.g. c-Abl and v-Abl), Ron receptor tyrosine kinases (“RON”), Met receptor tyrosine kinases (“MET”), Fms-like tyrosine kinases (“FLT”) (e.g. FLT3), src-family tyrosine kinases (e.g. lyn, CSK), FLT3, aurora-A kinases, B-lymphoid tyrosine kinases (“Blk”), src-family related protein tyrosine kinases (e.g. Fyn kinase), lymphocyte protein tyrosine kinases (“Lck”), nerve growth factor receptor (TRKC), sperm tyrosine kinases (e.g. Yes), Colony stimulating factor 1 receptor (CSF1R), vascular endothelial growth factor receptor 2 (VEGFR2, KDR), and many other important targets (see for example, Blume-Jensen P, Hunter T. “Oncogenic kinase signaling” Nature 2001, 411, 355-65) and subtypes and homologs thereof exhibiting tyrosine kinase activity. In certain embodiments, the protein tyrosine kinase is Abl, RON, MET, or AurA. In other embodiments, the protein tyrosine kinase is a MET or AurA family member.

In certain embodiments, the protein kinase is a protein serine/threonine kinase. A protein serine/threonine kinase, as used herein, refers to an enzyme that catalyzes the phosphorylation of serine and/or threonine residues in proteins with a phosphate donor (e.g. a nucleotide phosphate donor such as ATP). Protein serine/threonine kinases include, for example, p21-activated kinase-4 (“PAK”), cyclin-dependent kinases (“CDK”) (e.g. CDK1 and CDK5), glycogen synthase kinases (“GSK”) (e.g. GSK3α and GSK3β, ribosomal S6 kinases (e.g. Rsk1, Rsk2, and Rsk3), Raf kinases (e.g. BRAF, c-Raf), Akt (Protein kinase B, PKB) kinases, ROCK kinases, CHK kinases (CHK1, CHK2), polo kinases (e.g. PLK1), p38 kinases, other mitogen activated protein kinases (e.g. ERK1, ERK2, JNK), MAPK/ERK kinases (e.g. MEK), and subtypes and homologs thereof exhibiting serine/threonine kinase activity.

In another embodiment, the kinase is a mutant kinase, such as a mutant MET, Abl kinase or FLT3 kinase. Useful MET mutant kinases include Arg988Cys, Thr1010Ile, Tyr1253Asp, Asp1246Asn, Tyr1248Cys/His/Leu, Met1268Thr. Useful mutant Abl kinases include, for example, Bcr-Abl and Abl kinases having one of more of the following mutations: Glu255Lys, Thr315Ile, Tyr293Phe, or Met351Thr. In some embodiments, the mutant Abl kinase has a Y393F mutation or a T315I mutation. In another exemplary embodiment, the mutant Abl kinase has a Thr315Ile mutation.

In some embodiments, the kinase is homologous to a known kinase (also referred to herein as a “homologous kinase”). Compounds and compositions useful for inhibiting the biological activity of homologous kinases may be initially screened, for example, in binding assays. Homologous enzymes comprise an amino acid sequence of the same length that is at least 50%, at least 60%, at least 70%, at least 80%, or at least 90% identical to the amino acid sequence of full length known kinase, or 70%, 80%, or 90% homology to the known kinase active domains. Homology may be determined using, for example, a PSI BLAST search, such as, but not limited to that described in Altschul, et al., Nuc. Acids Rec. 25:3389-3402 (1997). In certain embodiments, at least 50%, or at least 70% of the sequence is aligned in this analysis. Other tools for performing the alignment include, for example, DbClustal and ESPript, which may be used to generate the PostScript version of the alignment. See Thompson et al., Nucleic Acids Research, 28:2919-26, 2000; Gouet, et al., Bioinformatics, 15:305-08 (1999). Homologs may, for example, have a BLAST E-value of 1×10⁻⁶ over at least 100 amino acids (Altschul et al., Nucleic Acids Res., 25:3389-402 (1997) with FLT3, Abl, or another known kinase, or any functional domain of FLT3, Abl, or another known kinase.

Homology may also be determined by comparing the active site binding pocket of the enzyme with the active site binding pockets of a known kinase. For example, in homologous enzymes, at least 50%, 60%, 70%, 80%, or 90% of the amino acids of the molecule or homolog have amino acid structural coordinates of a domain comparable in size to the kinase domain that have a root mean square deviation of the alpha carbon atoms of up to about 1.5 Å, about 1.25 Å, about 1 Å, about 0.75 Å, about 0.5 Å, and or about 0.25 Å.

The compounds and compositions of the present invention are useful for inhibiting kinase activity and also for inhibiting other enzymes that bind ATP. They are thus useful for the treatment of diseases and disorders that may be alleviated by inhibiting such ATP-binding enzyme activity. Methods of determining such ATP binding enzymes include those known to those of skill in the art, those discussed herein relating to selecting homologous enzymes, and by the use of the database PROSITE, where enzymes containing signatures, sequence patterns, motifs, or profiles of protein families or domains may be identified.

The compounds of the present invention, and their derivatives, may also be used as kinase-binding agents. As binding agents, such compounds and derivatives may be bound to a stable resin as a tethered substrate for affinity chromatography applications. The compounds of this invention, and their derivatives, may also be modified (e.g., radiolabelled or affinity labelled, etc.) in order to utilize them in the investigation of enzyme or polypeptide characterization, structure, and/or function.

In an exemplary embodiment, the pyrazolothiazole kinase modulator of the present invention is a kinase inhibitor. In some embodiments, the kinase inhibitor has an IC₅₀ of inhibition constant (Kᵢ) of less than 1 micromolar. In another embodiment, the kinase inhibitor has an IC₅₀ or inhibition constant (Kᵢ) of less than 500 micromolar. In another embodiment, the kinase inhibitor has an IC₅₀ or Kᵢ of less than 10 micromolar. In another embodiment, the kinase inhibitor has an IC₅₀ or Kᵢ of less than 1 micromolar. In another embodiment, the kinase inhibitor has an IC₅₀ or Kᵢ of less than 500 nanomolar. In another embodiment, the kinase inhibitor has an IC₅₀ or Kᵢ of less than 10 nanomolar. In another embodiment, the kinase inhibitor has an IC₅₀ or Kᵢ of less than 1 nanomolar.

I. Methods of Treatment

In another aspect, the present invention provides methods of treating a disease mediated by kinase activity (kinase-mediated disease or disorder) in an organism (e.g. mammals, such as humans). By “kinase-mediated” or “kinase-associated” diseases is meant diseases in which the disease or symptom can be alleviated by inhibiting kinase activity (e.g. where the kinase is involved in signaling, mediation, modulation, or regulation of the disease process). By “diseases” is meant diseases, or disease symptoms.

Examples of kinase associated diseases include cancer (e.g. leukemia, tumors, and metastases), allergy, asthma, inflammation (e.g. inflammatory airways disease), obstructive airways disease, autoimmune diseases, metabolic diseases, infection (e.g. bacterial, viral, yeast, fungal), CNS diseases, obesity, hematological disorders, bone disorders, brain tumors, degenerative neural diseases, cardiovascular diseases, and diseases associated with angiogenesis, neovascularization, and vasculogenesis. In an exemplary embodiment, the compounds are useful for treating cancer, including leukemia, and other diseases or disorders involving abnormal cell proliferation, myeloproliferative disorders, hematological disorders, asthma, inflammatory diseases or obesity.

More specific examples of cancers treated with the compounds of the present invention include breast cancer, lung cancer, melanoma, colorectal cancer, bladder cancer, ovarian cancer, prostate cancer, renal cancer, squamous cell cancer, glioblastoma, pancreatic cancer, Kaposi's sarcoma, multiple myeloma, and leukemia (e.g. myeloid, chronic myeloid, acute lymphoblastic, chronic lymphoblastic, Hodgkins, and other leukemias and hematological cancers).

Other specific examples of diseases or disorders for which treatment by the compounds or compositions of the invention are useful for treatment or prevention include, but are not limited to transplant rejection (for example, kidney, liver, heart, lung, islet cells, pancreas, bone marrow, cornea, small bowel, skin allografts or xenografts and other transplants), graft vs. host disease, osteoarthritis, rheumatoid arthritis, multiple sclerosis, diabetes, diabetic retinopathy, inflammatory bowel disease (for example, Crohn's disease, ulcerative colitis, and other bowel diseases), renal disease, cachexia, septic shock, lupus, myasthenia gravis, psoriasis, dermatitis, eczema, seborrhea, Alzheimer's disease, Parkinson's disease, stem cell protection during chemotherapy, ex vivo selection or ex vivo purging for autologous or allogeneic bone marrow transplantation, ocular disease, retinopathies (for example, macular degeneration, diabetic retinopathy, and other retinopathies), corneal disease, glaucoma, infections (for example bacterial, viral, or fungal), heart disease, including, but not limited to, restenosis.

Assays

The compounds of the present invention may be easily assayed to determine their ability to modulate protein kinases, bind protein kinases, and/or prevent cell growth or proliferation. Some examples of useful assays are presented below.

Kinase Inhibition and Binding Assays

Inhibition of various kinases is measured by methods known to those of ordinary skill in the art, such as the various methods presented herein, and those discussed in the Upstate KinaseProfiler Assay Protocols June 2003 publication.

For example, where in vitro assays are performed, the kinase is typically diluted to the appropriate concentration to form a kinase solution. A kinase substrate and phosphate donor, such as ATP, is added to the kinase solution. The kinase is allowed to transfer a phosphate to the kinase substrate to form a phosphorylated substrate. The formation of a phosphorylated substrate may be detected directly by any appropriate means, such as radioactivity (e.g. [γ-³²P-ATP]), or the use of detectable secondary antibodies (e.g. ELISA). Alternatively, the formation of a phosphorylated substrate may be detected using any appropriate technique, such as the detection of ATP concentration (e.g. Kinase-Glo® assay system (Promega)). Kinase inhibitors are identified by detecting the formation of a phosphorylated substrate in the presence and absence of a test compound (see Examples section below).

The ability of the compound to inhibit a kinase in a cell may also be assayed using methods well known in the art. For example, cells containing a kinase may be contacted with an activating agent (such as a growth factor) that activates the kinase. The amount of intracellular phosphorylated substrate formed in the absence and the presence of the test compound may be determined by lysing the cells and detecting the presence of phosphorylated substrate by any appropriate method (e.g. ELISA). Where the amount of phosphorylated substrate produced in the presence of the test compound is decreased relative to the amount produced in the absence of the test compound, kinase inhibition is indicated. More detailed cellular kinase assays are discussed in the Examples section below.

To measure the binding of a compound to a kinase, any method known to those of ordinary skill in the art may be used. For example, a test kit manufactured by DiscoveRx (Fremont, Calif.), ED-Staurosporine NSIP™ Enzyme Binding Assay Kit (see U.S. Pat. No. 5,643,734) may be used. Kinase activity may also be assayed as in U.S. Pat. No. 6,589,950, issued Jul. 8, 2003.

Suitable kinase inhibitors may be selected from the compounds of the invention through protein crystallographic screening, as disclosed in, for example Antonysamy, et al., PCT Publication No. WO03087816A1, which is incorporate herein by reference in its entirety for all purposes.

The compounds of the present invention may be computationally screened to assay and visualize their ability to bind to and/or inhibit various kinases. The structure may be computationally screened with a plurality of compounds of the present invention to determine their ability to bind to a kinase at various sites. Such compounds can be used as targets or leads in medicinal chemistry efforts to identify, for example, inhibitors of potential therapeutic importance (Travis, Science, 262:1374, 1993). The three dimensional structures of such compounds may be superimposed on a three dimensional representation of kinases or an active site or binding pocket thereof to assess whether the compound fits spatially into the representation and hence the protein. In this screening, the quality of fit of such entities or compounds to the binding pocket may be judged either by shape complementarity or by estimated interaction energy (Meng, et al., J. Comp. Chem. 13:505-24, 1992).

The screening of compounds of the present invention that bind to and/or modulate kinases (e.g. inhibit or activate kinases) according to this invention generally involves consideration of two factors. First, the compound must be capable of physically and structurally associating, either covalently or non-covalently with kinases. For example, covalent interactions may be important for designing irreversible or suicide inhibitors of a protein. Non-covalent molecular interactions important in the association of kinases with the compound include hydrogen bonding, ionic interactions, van der Waals, and hydrophobic interactions. Second, the compound must be able to assume a conformation and orientation in relation to the binding pocket, that allows it to associate with kinases. Although certain portions of the compound will not directly participate in this association with kinases, those portions may still influence the overall conformation of the molecule and may have a significant impact on potency. Conformational requirements include the overall three-dimensional structure and orientation of the chemical group or compound in relation to all or a portion of the binding pocket, or the spacing between functional groups of a compound comprising several chemical groups that directly interact with kinases.

Docking programs described herein, such as, for example, DOCK, or GOLD, are used to identify compounds that bind to the active site and/or binding pocket. Compounds may be screened against more than one binding pocket of the protein structure, or more than one set of coordinates for the same protein, taking into account different molecular dynamic conformations of the protein. Consensus scoring may then be used to identify the compounds that are the best fit for the protein (Charifson, P. S. et al., J. Med. Chem. 42: 5100-9 (1999)). Data obtained from more than one protein molecule structure may also be scored according to the methods described in Klingler et al., U.S. Utility Application, filed May 3, 2002, entitled “Computer Systems and Methods for Virtual Screening of Compounds.” Compounds having the best fit are then obtained from the producer of the chemical library, or synthesized, and used in binding assays and bioassays.

Computer modeling techniques may be used to assess the potential modulating or binding effect of a chemical compound on kinases. If computer modeling indicates a strong interaction, the molecule may then be synthesized and tested for its ability to bind to kinases and affect (by inhibiting or activating) its activity.

Modulating or other binding compounds of kinases may be computationally evaluated by means of a series of steps in which chemical groups or fragments are screened and selected for their ability to associate with the individual binding pockets or other areas of kinases. This process may begin by visual inspection of, for example, the active site on the computer screen based on the kinases coordinates. Selected fragments or chemical groups may then be positioned in a variety of orientations, or docked, within an individual binding pocket of kinases (Blaney, J. M. and Dixon, J. S., Perspectives in Drug Discovery and Design, 1:301, 1993). Manual docking may be accomplished using software such as Insight II (Accelrys, San Diego, Calif.) MOE (Chemical Computing Group, Inc., Montreal, Quebec, Canada); and SYBYL (Tripos, Inc., St. Louis, Mo., 1992), followed by energy minimization and/or molecular dynamics with standard molecular mechanics force fields, such as CHARMM (Brooks, et al., J. Comp. Chem. 4:187-217, 1983), AMBER (Weiner, et al., J. Am. Chem. Soc. 106: 765-84, 1984) and C² MMFF (Merck Molecular Force Field; Accelrys, San Diego, Calif.). More automated docking may be accomplished by using programs such as DOCK (Kuntz et al., J. Mol. Biol., 161:269-88, 1982; DOCK is available from University of California, San Francisco, Calif.); AUTODOCK (Goodsell & Olsen, Proteins: Structure, Function, and Genetics 8:195-202, 1990; AUTODOCK is available from Scripps Research Institute, La Jolla, Calif.); GOLD (Cambridge Crystallographic Data Centre (CCDC); Jones et al., J. Mol. Biol. 245:43-53, 1995); and FLEXX (Tripos, St. Louis, Mo.; Rarey, M., et al., J. Mol. Biol. 261:470-89, 1996). Other appropriate programs are described in, for example, Halperin, et al.

During selection of compounds by the above methods, the efficiency with which that compound may bind to kinases may be tested and optimized by computational evaluation. For example, a compound that has been designed or selected to function as a kinases inhibitor may occupy a volume not overlapping the volume occupied by the active site residues when the native substrate is bound, however, those of ordinary skill in the art will recognize that there is some flexibility, allowing for rearrangement of the main chains and the side chains. In addition, one of ordinary skill may design compounds that could exploit protein rearrangement upon binding, such as, for example, resulting in an induced fit. An effective kinase inhibitor may demonstrate a relatively small difference in energy between its bound and free states (i.e., it must have a small deformation energy of binding and/or low conformational strain upon binding). Thus, the most efficient kinase inhibitors should, for example, be designed with a deformation energy of binding of not greater than 10 kcal/mol, not greater than 7 kcal/mol, not greater than 5 kcal/mol, or not greater than 2 kcal/mol. Kinase inhibitors may interact with the protein in more than one conformation that is similar in overall binding energy. In those cases, the deformation energy of binding is taken to be the difference between the energy of the free compound and the average energy of the conformations observed when the inhibitor binds to the enzyme.

Specific computer software is available in the art to evaluate compound deformation energy and electrostatic interaction. Examples of programs designed for such uses include: Gaussian 94, revision C (Frisch, Gaussian, Inc., Pittsburgh, Pa. ©1995); AMBER, version 7. (Kollman, University of California at San Francisco, ©2002); QUANTA/CHARMM (Accelrys, Inc., San Diego, Calif., ©1995); Insight II/Discover (Accelrys, Inc., San Diego, Calif., ©1995); DelPhi (Accelrys, Inc., San Diego, Calif., ©1995); and AMSOL (University of Minnesota) (Quantum Chemistry Program Exchange, Indiana University). These programs may be implemented, for instance, using a computer workstation, as are well known in the art, for example, a LINUX, SGI or Sun workstation. Other hardware systems and software packages will be known to those skilled in the art.

Those of ordinary skill in the art may express kinase protein using methods known in the art, and the methods disclosed herein. The native and mutated kinase polypeptides described herein may be chemically synthesized in whole or part using techniques that are well known in the art (see, e.g., Creighton, Proteins: Structures and Molecular Principles, W. H. Freeman & Co., NY, 1983).

Gene expression systems may be used for the synthesis of native and mutated polypeptides. Expression vectors containing the native or mutated polypeptide coding sequence and appropriate transcriptional/translational control signals, that are known to those skilled in the art may be constructed. These methods include in vitro recombinant DNA techniques, synthetic techniques and in vivo recombination/genetic recombination. See, for example, the techniques described in Sambrook et al., Molecular Cloning: A Laboratory Manual, Cold Spring Harbor Laboratory, NY, 2001, and Ausubel et al., Current Protocols in Molecular Biology, Greene Publishing Associates and Wiley Interscience, NY, 1989.

Host-expression vector systems may be used to express kinase. These include, but are not limited to, microorganisms such as bacteria transformed with recombinant bacteriophage DNA, plasmid DNA or cosmid DNA expression vectors containing the coding sequence; yeast transformed with recombinant yeast expression vectors containing the coding sequence; insect cell systems infected with recombinant virus expression vectors (e.g., baculovirus) containing the coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the coding sequence; or animal cell systems. The protein may also be expressed in human gene therapy systems, including, for example, expressing the protein to augment the amount of the protein in an individual, or to express an engineered therapeutic protein. The expression elements of these systems vary in their strength and specificities.

Specifically designed vectors allow the shuttling of DNA between hosts such as bacteria-yeast or bacteria-animal cells. An appropriately constructed expression vector may contain: an origin of replication for autonomous replication in host cells, one or more selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one that causes mRNAs to be initiated at high frequency.

The expression vector may also comprise various elements that affect transcription and translation, including, for example, constitutive and inducible promoters. These elements are often host and/or vector dependent. For example, when cloning in bacterial systems, inducible promoters such as the T7 promoter, pL of bacteriophage λ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, mammalian promoters (e.g., metallothionein promoter) or mammalian viral promoters, (e.g., adenovirus late promoter; vaccinia virus 7.5K promoter; SV40 promoter; bovine papilloma virus promoter; and Epstein-Barr virus promoter) may be used.

Various methods may be used to introduce the vector into host cells, for example, transformation, transfection, infection, protoplast fusion, and electroporation. The expression vector-containing cells are clonally propagated and individually analyzed to determine whether they produce the appropriate polypeptides. Various selection methods, including, for example, antibiotic resistance, may be used to identify host cells that have been transformed. Identification of polypeptide expressing host cell clones may be done by several means, including but not limited to immunological reactivity with anti- kinase antibodies, and the presence of host cell-associated activity.

Expression of cDNA may also be performed using in vitro produced synthetic mRNA. Synthetic mRNA can be efficiently translated in various cell-free systems, including but not limited to wheat germ extracts and reticulocyte extracts, as well as efficiently translated in cell-based systems, including, but not limited to, microinjection into frog oocytes.

To determine the cDNA sequence(s) that yields optimal levels of activity and/or protein, modified cDNA molecules are constructed. A non-limiting example of a modified cDNA is where the codon usage in the cDNA has been optimized for the host cell in which the cDNA will be expressed. Host cells are transformed with the cDNA molecules and the levels of kinase RNA and/or protein are measured.

Levels of kinase protein in host cells are quantitated by a variety of methods such as immunoaffinity and/or ligand affinity techniques, kinase-specific affinity beads or specific antibodies are used to isolate ³⁵S-methionine labeled or unlabeled protein. Labeled or unlabeled protein is analyzed by SDS-PAGE. Unlabeled protein is detected by Western blotting, ELISA or RIA employing specific antibodies.

Following expression of kinase in a recombinant host cell, polypeptides may be recovered to provide the protein in active form. Several purification procedures are available and suitable for use. Recombinant kinase may be purified from cell lysates or from conditioned culture media, by various combinations of, or individual application of, fractionation, or chromatography steps that are known in the art.

In addition, recombinant kinase can be separated from other cellular proteins by use of an immuno-affinity column made with monoclonal or polyclonal antibodies specific for full length nascent protein or polypeptide fragments thereof. Other affinity based purification techniques known in the art may also be used.

Alternatively, the polypeptides may be recovered from a host cell in an unfolded, inactive form, e.g., from inclusion bodies of bacteria. Proteins recovered in this form may be solubilized using a denaturant, e.g., guanidinium hydrochloride, and then refolded into an active form using methods known to those skilled in the art, such as dialysis.

Cell Growth Assays

A variety of cell growth assays are known in the art and are useful in identifying pyrazolothiazole compounds (i.e. “test compounds”) capable of inhibiting (e.g. reducing) cell growth and/or proliferation.

For example, a variety of cells are known to require specific kinases for growth and/or proliferation. The ability of such a cell to grow in the presence of a test compound may be assessed and compared to the growth in the absence of the test compound thereby identifying the anti-proliferative properties of the test compound. One common method of this type is to measure the degree of incorporation of label, such as tritiated thymidine, into the DNA of dividing cells. Alternatively, inhibition of cell proliferation may be assayed by determining the total metabolic activity of cells with a surrogate marker that correlates with cell number. Cells may be treated with a metabolic indicator in the presence and absence of the test compound. Viable cells metabolize the metabolic indicator thereby forming a detectable metabolic product. Where detectable metabolic product levels are decreased in the presence of the test compound relative to the absence of the test compound, inhibition of cell growth and/or proliferation is indicated. Exemplary metabolic indicators include, for example tetrazolium salts and AlamorBlue® (see Examples section below).

Pharmaceutical Compositions and Administration

In another aspect, the present invention provides a pharmaceutical composition including a pyrazolothiazole kinase modulator in admixture with a pharmaceutically acceptable excipient. One of skill in the art will recognize that the pharmaceutical compositions include the pharmaceutically acceptable salts of the pyrazolothiazole kinase modulators described above.

In therapeutic and/or diagnostic applications, the compounds of the invention can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A most preferable dosage is 10 to 30 mg per day. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, such agents may be formulated into liquid or solid dosage forms and administered systemically or locally. The agents may be delivered, for example, in a timed- or sustained- low release form as is known to those skilled in the art. Techniques for formulation and administration may be found in Remington: The Science and Practice of Pharmacy (₂₀th ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra -sternal, intra-synovial, intra-hepatic, intralesional, intracranial, intraperitoneal, intranasal, or intraocular injections or other modes of delivery.

For injection, the agents of the invention may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

For nasal or inhalation delivery, the agents of the invention may also be formulated by methods known to those of skill in the art, and may include, for example, but not limited to, examples of solubilizing, diluting, or dispersing substances such as, saline, preservatives, such as benzyl alcohol, absorption promoters, and fluorocarbons.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Depending upon the particular condition, or disease state, to be treated or prevented, additional therapeutic agents, which are normally administered to treat or prevent that condition, may be administered together with the inhibitors of this invention. For example, chemotherapeutic agents or other anti-proliferative agents may be combined with the inhibitors of this invention to treat proliferative diseases and cancer. Examples of known chemotherapeutic agents include, but are not limited to, adriamycin, dexamethasone, vincristine, cyclophosphamide, fluorouracil, topotecan, taxol, interferons, and platinum derivatives.

Other examples of agents the inhibitors of this invention may also be combined with include, without limitation, anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents for treating diabetes such as insulin, insulin analogues, alpha glucosidase inhibitors, biguanides, and insulin sensitizers; and agents for treating immunodeficiency disorders such as gamma globulin.

These additional agents may be administered separately, as part of a multiple dosage regimen, from the inhibitor-containing composition. Alternatively, these agents may be part of a single dosage form, mixed together with the inhibitor in a single composition.

The present invention is not to be limited in scope by the exemplified embodiments, which are intended as illustrations of single aspects of the invention. Indeed, various modifications of the invention in addition to those described herein will become apparent to those having skill in the art from the foregoing description. Such modifications are intended to fall within the scope of the invention. Moreover, any one or more features of any embodiment of the invention may be combined with any one or more other features of any other embodiment of the invention, without departing from the scope of the invention. For example, the pyrazolothiazole kinase modulators described in the Pyrazolothiazole Kinase Modulators section are equally applicable to the methods of treatment and methods of inhibiting kinases described herein. References cited throughout this application are examples of the level of skill in the art and are hereby incorporated by reference herein in their entirety for all purposes, whether previously specifically incorporated or not.

### EXAMPLES

The following examples are offered to illustrate, but not to limit the claimed invention. The preparation of embodiments of the present invention is described in the following examples. Those of ordinary skill in the art will understand that the chemical reactions and synthesis methods provided may be modified to prepare many of the other compounds of the present invention. Where compounds of the present invention have not been exemplified, those of ordinary skill in the art will recognize that these compounds may be prepared by modifying synthesis methods presented herein, and by using synthesis methods known in the art.

### Example 1

### Synthesis of Compounds

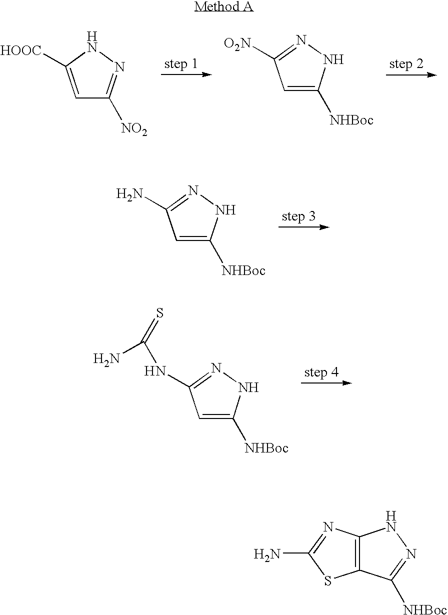

### Step 1: Synthesis of (5-nitro-2H-pyrazol-3-yl)-carbamic acid tert-butyl ester

A suspension of 5-nitro-2H-pyrazole-3-carboxylic acid (10.35 g, 68.96 mmol) in tert-BuOH (40 mL) was treated with triethylamine (19.25 ml, 137.92 mmol), followed by diphenylphosphorylazide (30 ml, 137.92 mmol). The mixture was heated to reflux for 16 hours. The solution was diluted with EtOAc and washed with water twice. The aqueous layer was extracted with EtOAc and the combined organic layers were washed with brine, dried over Na₂SO₄, filtered and concentrated in vacuo. The crude residue was triturated with dichloromethane to afford 10.43 g of (5-nitro-2H-pyrazol-3-yl)-carbamic acid tert-butyl ester as a solid (66% yield). ¹H NMR (d₆-DMSO) δ 13.5 (1H, s), 10.4 (1H, broad s), 6.44 (1H, s), 1.48 (9H, s).

### Step 2: Synthesis of (5-amino-2H-pyrazol-3-yl)-carbamic acid tert-butyl ester

(5-Nitro-2H-pyrazol-3-yl)-carbamic acid tert-butyl ester (10.4 g, 45.61 mmol) was placed in a hydrogenation vessel and dissolved in methanol (150 ml). The solution was purged with nitrogen gas and 10% palladium on carbon (1.02 g, 0.958 mmol) was added to the reaction vessel while maintaining an inert environment. The vessel was placed on the Parr hydrogenator overnight. The reaction mixture was filtered over celite and concentrated under vacuo to afford 9.0 g of (5-amino-2H-pyrazol-3-yl)-carbamic acid tert-butyl ester as a foam (quantitative yield). ¹H NMR (d₆-DMSO) δ 10.9 (1H, s), 9.25 (1H, broad s), 5.57 (1H, s), 5.10 (2H, s) 1.64 (9H, s); HPLC/MS m/z: 199 [MH]⁺.

### Step 3: Synthesis of (5-thioureido-2H-pyrazol-3-yl)-carbamic acid tert-butyl ester

To a solution of (5-amino-2H-pyrazol-3-yl)-carbamic acid tert-butyl ester (14.48 g, 73.13 mmol) in THF (110 mL) was added benzoylisothiocyanate (10.8 ml, 80.44 mmol) dropwise. The reaction mixture was stirred at room temperature until completion, then 4 N aqueous NaOH (110 mL) was added. The reaction mixture was stirred at 40° C. for 6 h, before dilution with EtOAc. The organics were washed with 1 N aqueous HCl and brine, then dried over Na₂SO₄, filtered, and concentrated in vacuo. The crude residue was triturated with diethyl ether, and the precipitate was filtered and dried in vacuo to afford 15.1 g of (5-thioureido-2H-pyrazol-3-yl)-carbamic acid tert-butyl ester (80% yield). ¹H NMR (d₆-DMSO) δ 11.8 (1H, s), 10.2 (1H, s), 10.0 (1H, s) 9.11 (1H, s) 8.46 (1H, s) 5.53 (1H, s), 1.46 (9H, s); HPLC/MS m/z: 258 [MH]⁺.

### Step 4: Synthesis of (5-amino-1H-pyrazol-[3,4-d]thiazol-3-yl)-carbamic acid tert-butyl ester

A 1.5 M solution of bromine in acetic acid (0.46 mL, 89.78 mmol) was added dropwise to a solution of (5-thioureido-2H-pyrazol-3-yl)-carbamic acid tert-butyl ester (22.0 g, 85.50 mmol) in acetic acid (1.71 L), while stirring vigorously. Upon completion of the addition, the reaction mixture was immediately concentrated in vacuo to afford a solid, to which a saturated solution of sodium bicarbonate was added slowly until pH 8. The resulting precipitate was filtered and dried in vacuo to afford 16.08 g of (5-amino-1H-pyrazol-[3,4-d]thiazol-3-yl)-carbamic acid tert-butyl ester as a white solid (73% yield): ¹H NMR (d₆-DMSO) δ 11.9 (broad s, 1H), 9.74 (broad s, 1H), 7.27 (broad s, 2H), 1.42 (s, 9H); HPLC/MS m/z: 256 [MH]⁺.

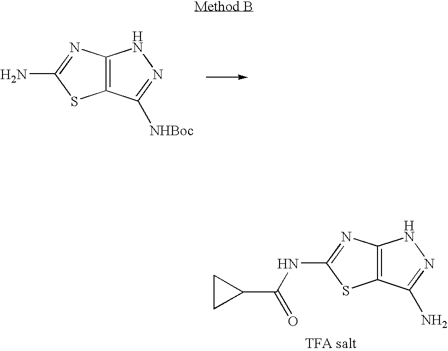

### Synthesis of cyclopropanecarboxylic acid (3-amino-1H-pyrazolo[3,4-d]thiazol-5-yl)-amide, trifluoroacetic acid salt

To a solution of (5-amino-1H-pyrazol-[3,4-d]thiazol-3-yl)-carbamic acid tert-butyl ester (16.0 g, 62.7 mmol) in THF (313 mL) was added pyridine (30.4 ml, 376 mmol), followed by cyclopropanecarbonyl chloride (29.0 ml, 313.3 mmol) dropwise. The reaction mixture was stirred at 70° C. for 3 h, then N,N-dimethylethylenediamine (44.6 ml, 627 mmol) was added and the reaction mixture was stirred further at 70° C. for 1 h. The reaction mixture was concentrated in vacuo and redissolved in ethyl acetate. The organic layer was washed with copious amounts of 10% aqueous citric acid solution, dried over Na₂SO₄, filtered, and left standing overnight. The resulting precipitate was filtered and dried in vacuo to provide 18.3 g of [5-(cyclopropanecarbonyl-amino)-1H-pyrazolo[3,4-d]thiazol-3-yl]-carbamic acid tert-butyl ester (90% yield). ¹H NMR (d₆-DMSO) δ 12.4 (1H, s), 10.0 (1H, s), 1.44 (9H, s), 1.98 (1H, m), 0.95 (4H, m).

To a suspension of [5-(cyclopropanecarbonyl-amino)-1H-pyrazolo[3,4-d]thiazol-3-yl]-carbamic acid tert-butyl ester (17.0 g, 52.61 mmol) in dichloromethane (500 mL) was added trifluoroacetic acid (180 mL) dropwise. The reaction mixture was stirred at room temperature for 3 h, then concentrated in vacuo. The crude was triturated with Et₂O, filtered, and washed with Et₂O. Drying in vacuo provided 12.47 g of cyclopropanecarboxylic acid (3-amino-1H-pyrazolo[3,4-d]thiazol-5-yl)-amide, trifluoroacetic acid salt as a light beige solid (70% yield). ¹H NMR (d₆-DMSO) δ 12.8 (s, 1H), 1.97 (m, 1H), 0.94 (m, 4H); HPLC/MS m/z: 224 [MH]⁺.

Other compound prepared by method B:

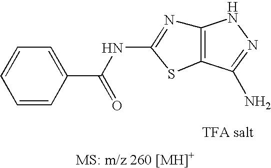

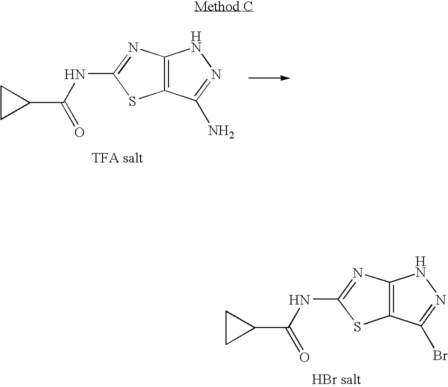

### Synthesis of cyclopropanecarboxylic acid (3-bromo-1H-pyrazolo[3,4-d]thiazol-5-yl)-amide, HBr salt

To a suspension of cyclopropanecarboxylic acid (3-amino-1H-pyrazolo[3,4-d]thiazol-5-yl)-amide, trifluoroacetic acid salt (622 mg, 1.84 mmol) in aqueous HBr (15 mL) was slowly added NaNO₂ (153 mg, 2.21 mmol). After stirring for 1 h, CuBr (742 mg, 5.17 mmol) was added and the reaction was heated at 40° C. for 17 h. The mixture was diluted with water and extracted with EtOAc (3×). The combined organics were washed with brine and dried over Na₂SO₄ and concentrated in vacuo. After drying on high vacuum, 381 mg of cyclopropanecarboxylic acid (3-bromo-1H-pyrazolo[3,4-d]thiazol-5-yl)-amide HBr salt was obtained as an off white solid (56% yield). HPLC/MS m/z: 287 [MH]⁺.

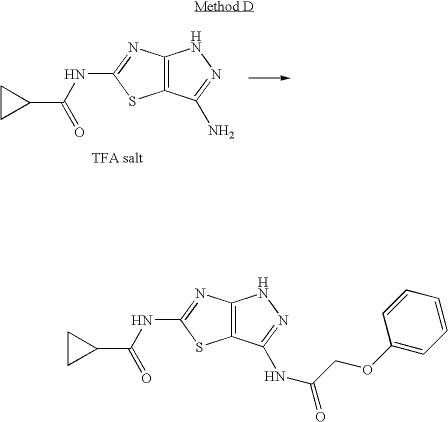

### Synthesis of cyclopropanecarboxylic acid [3-(2-phenoxy-acetylamino)-1H-pyrazolo[3,4-d]thiazol-5-yl]-amide

To a suspension of cyclopropanecarboxylic acid (3-amino-1H-pyrazolo[3,4-d]thiazol-5-yl)-amide, trifluoroacetic acid salt (20 mg, 0.059 mmol) in THF (0.5 mL) was added pyridine (0.032 mL, 0.393 mmol), followed by phenoxyacetyl chloride (0.041 mL, 0.295 mmol). The reaction mixture was stirred at 70° C. for 16 h, and then cooled to room temperature. N,N-Dimethylethylenediamine (0.1 mL) was added, and the reaction mixture was stirred at 70° C. for 2.5 h. After cooling at room temperature, the clear solution was adsorbed on silica gel. Purification on silica gel with 0-8% gradient of MeOH/CH₂Cl₂ as eluent provided 10 mg of cyclopropanecarboxylic acid [3-(2-phenoxy-acetylamino)-1H-pyrazolo[3,4-d]thiazol-5-yl]-amide as a white solid (57% yield). ¹H NMR (d₆-DMSO) δ 12.7 (broad s, 1H), 12.4 (broad s, 1H), 10.9 (broad s, 1H), 7.30 (t, 2H), 6.95 (m, 3H), 4.71 (s, 2H), 1.94 (m, 1H), 0.90 (m, 4H); HPLC/MS m/z: 358 [MH]⁺.

Other compounds prepared by method D:

TABLE 1

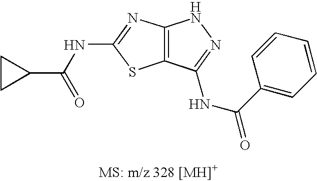

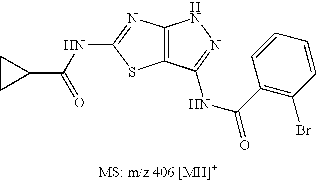

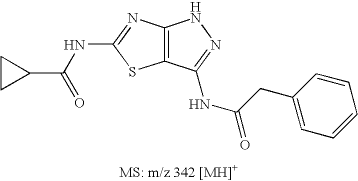

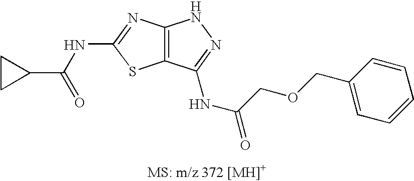

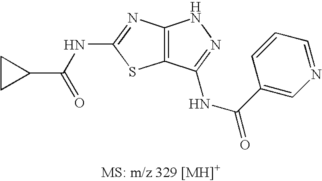

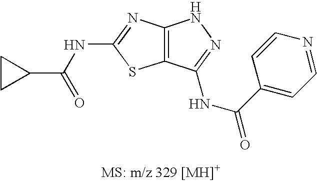

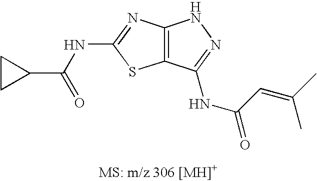

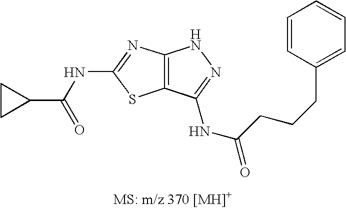

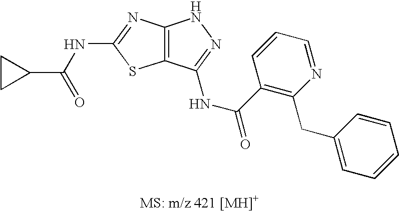

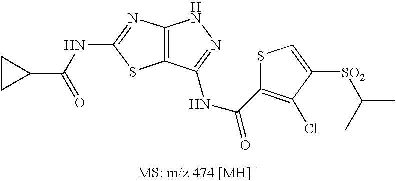

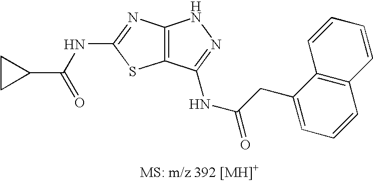

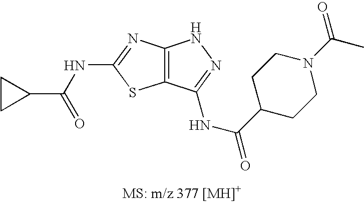

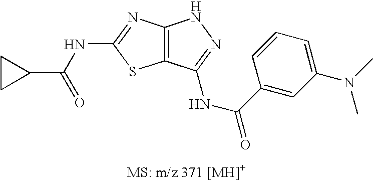

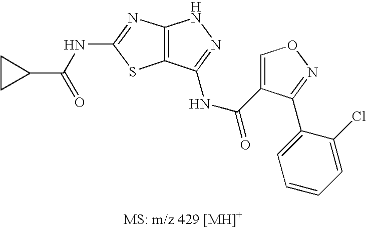

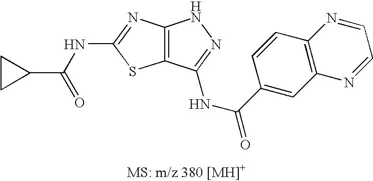

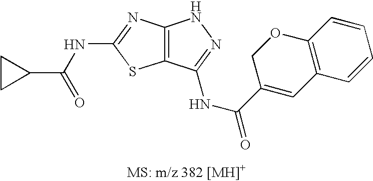

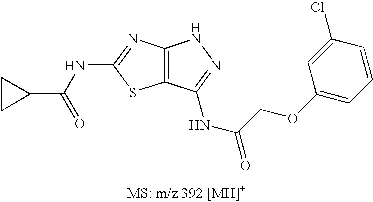

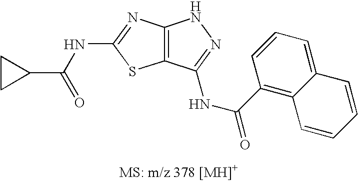

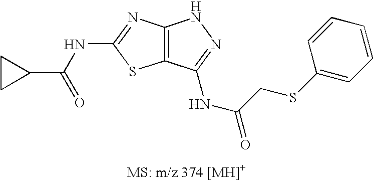

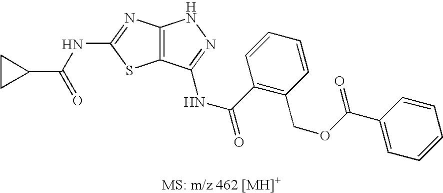

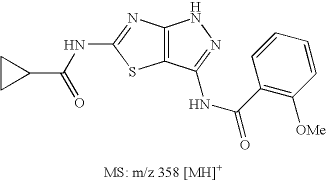

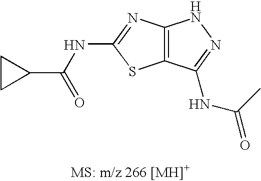

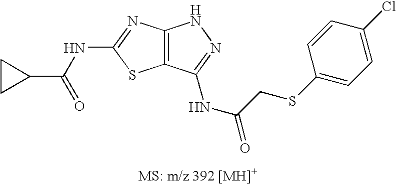

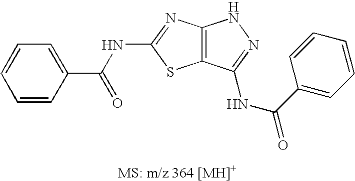

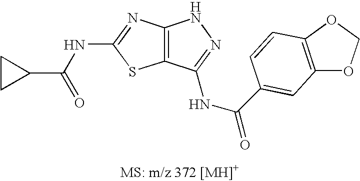

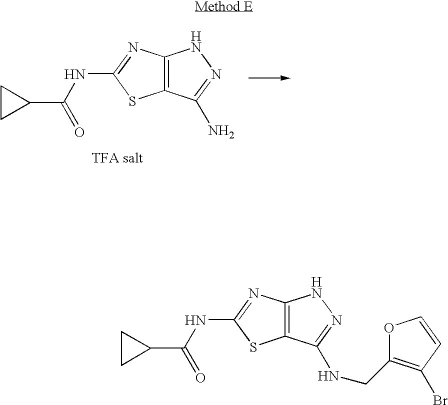

### Synthesis of cyclopropanecarboxylic acid {3-[(3-bromo-furan-2-ylmethyl)-amino]-1H-pyrazolo[3,4-d]thiazol-5-yl}-amide

To a solution of cyclopropanecarboxylic acid (3-amino-1H-pyrazolo[3,4-d]thiazol-5-yl)-amide, trifluoroacetic acid salt (20 mg, 0.059 mmol) in DMF/AcOH (3:1, 0.4 mL) was added 3-bromo-furan-2-carbaldehyde (12.4 mg, 0.071 mmol), followed by sodium cyanoborohydride (11 mg, 0.177 mmol). The reaction mixture was stirred at 40° C. for 4 h, and then concentrated in vacuo. The crude solid was triturated with a saturated aqueous solution of NaHCO₃ before extraction with EtOAc, and the extracts were adsorbed on silica gel. Purification on silica gel with 0-8% gradient of MeOH/CH₂Cl₂ as eluent provided 7.3 mg of cyclopropanecarboxylic acid {3-[(3-bromo-furan-2-ylmethyl)-amino]-1H-pyrazolo[3,4-d]thiazol-5-yl}-amide as an off-white solid (32% yield). ¹H NMR (d₆-DMSO) δ 12.4 (broad s, 1H), 11.8 (broad s, 1H), 6.45 (d, 1H), 6.30 (d, 1H), 6.21 (broad s, 1H), 4.26 (d, 2H), 1.93 (m, 1H), 0.90 (m, 4H); HPLC/MS m/z: 382 [MH]⁺.

Other compounds prepared by method E:

TABLE 2

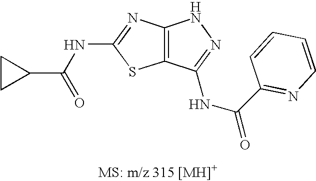

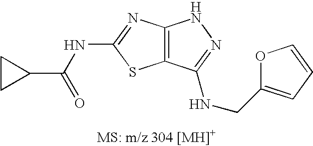

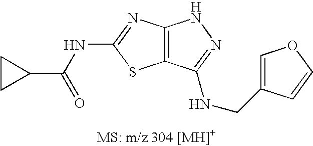

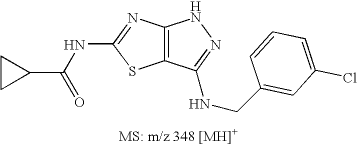

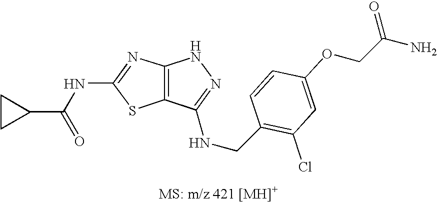

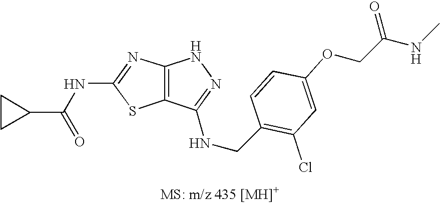

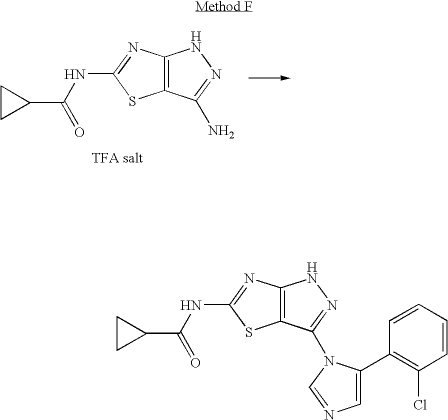

### Synthesis of cyclopropanecarboxylic acid {3-[5-(2-chloro-phenyl)-imidazol-1-yl]-1H-pyrazolo[3,4-d]thiazol-5-yl}-amide

To a suspension of cyclopropanecarboxylic acid (3-amino-1H-pyrazolo[3,4-d]thiazol-5-yl)-amide, trifluoroacetic acid salt (100 mg, 0.297 mmol) in absolute EtOH (2.5 mL) was added 2-chlorobenzaldehyde (0.04 mL, 0.356 mmol). The reaction mixture was refluxed for 16 h, and then it was dried in vacuo. The crude solid was dissolved in DMF (2.5 mL) under nitrogen atmosphere. Potassium carbonate (123 mg, 0.891 mmol) was added, followed by tosylmethyl isocyanide (58 mg, 0.297 mmol). The reaction mixture was stirred at 80° C. for 3 h, and then concentrated in vacuo. The crude solid was redissolved in 10% MeOH/CH₂Cl₂ and adsorbed on silica gel. Purification on silica gel with 0-8% gradient of MeOH/CH₂Cl₂ as eluent provided 52 mg of cyclopropanecarboxylic acid {3-[5-(2-chloro-phenyl)-imidazol-1-yl]-1H-pyrazolo[3,4-d]thiazol-5-yl}-amide as a cream-colored solid (45% yield). ¹H NMR (d₆-DMSO) δ 13.5 (broad s, 1H), 12.6 (broad s, 1H), 8.18 (s, 1H), 7.48 (d, 1H), 7.37-7.43 (m, 3H), 7.18 (s, 1H), 1.89 (m, 1H), 0.90 (m, 2H), 0.87 (m. 2H); HPLC/MS m/z: 385 [MH]⁺.

Other compounds prepared by method F:

TABLE 3

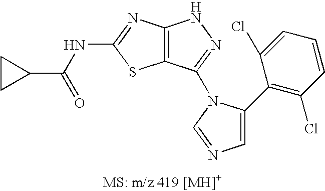

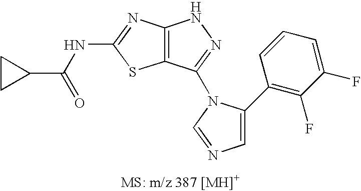

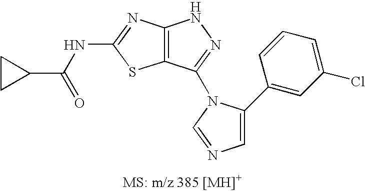

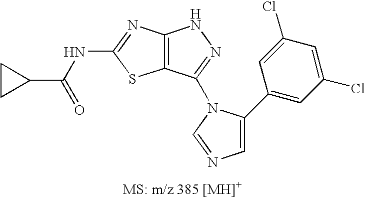

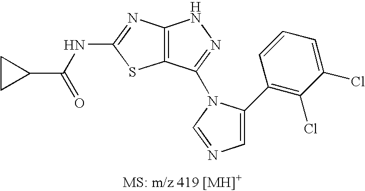

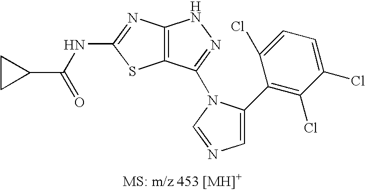

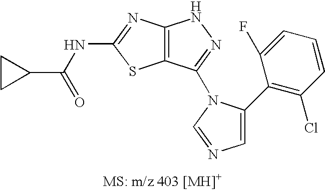

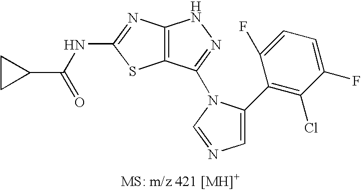

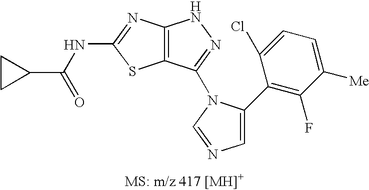

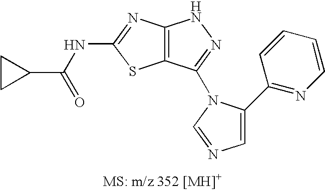

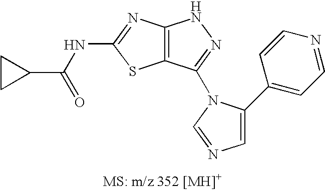

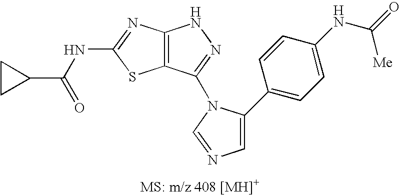

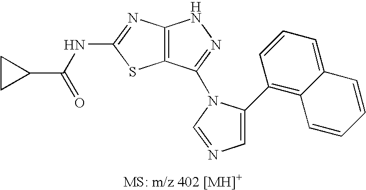

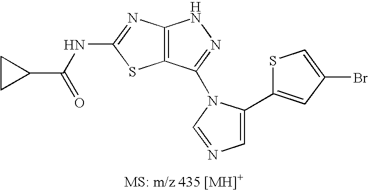

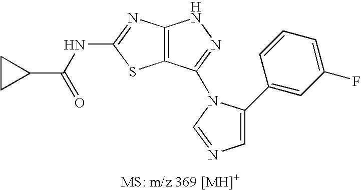

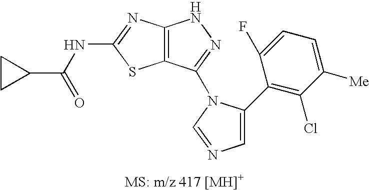

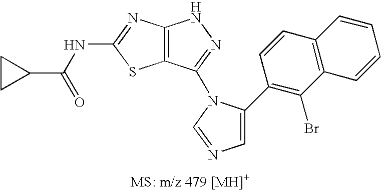

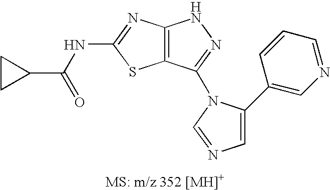

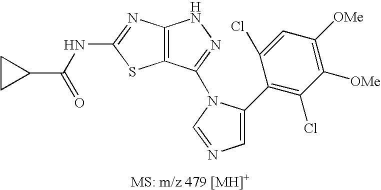

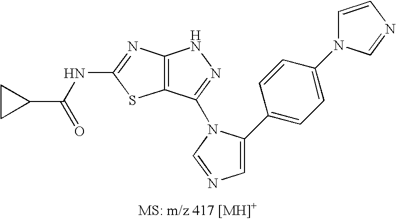

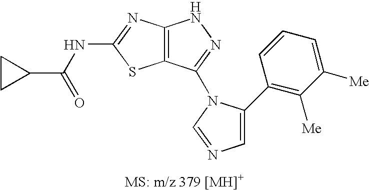

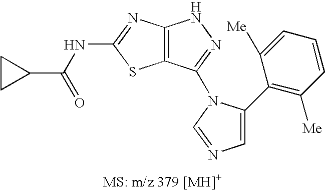

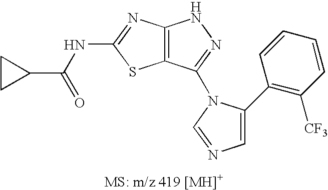

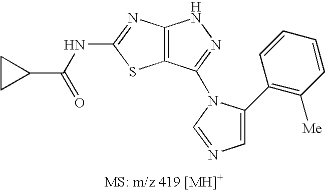

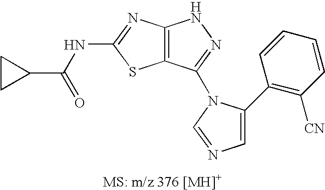

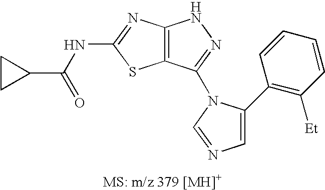

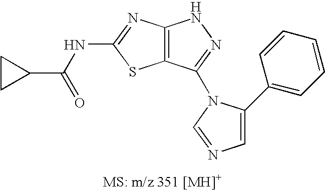

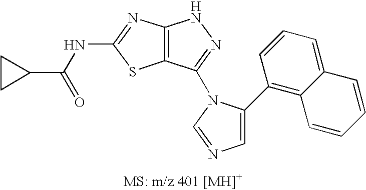

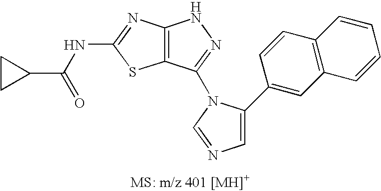

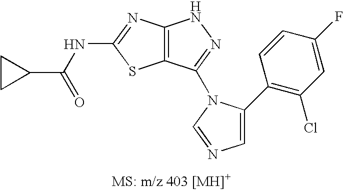

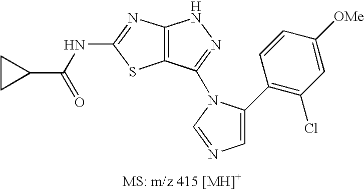

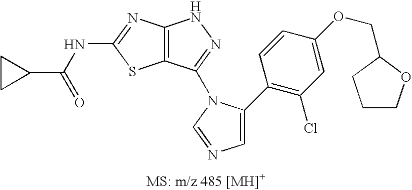

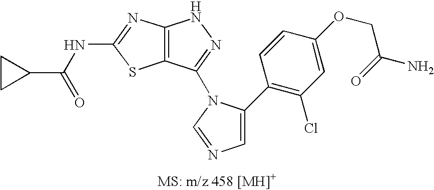

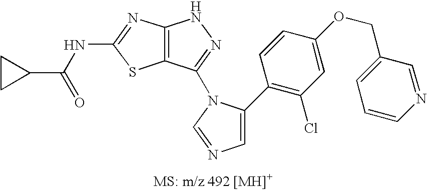

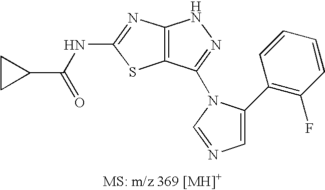

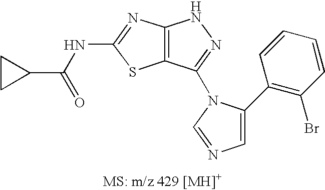

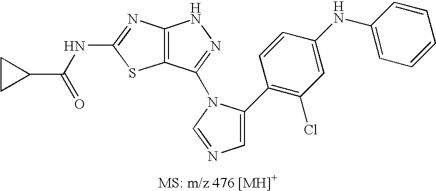

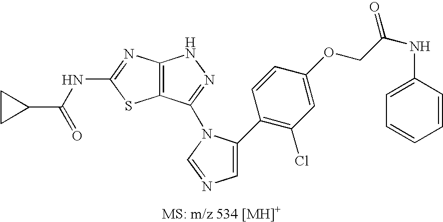

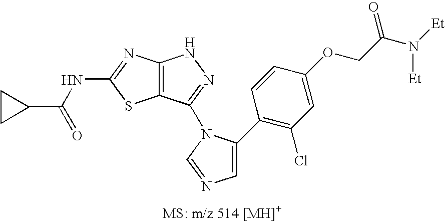

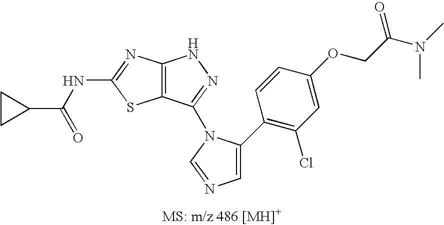

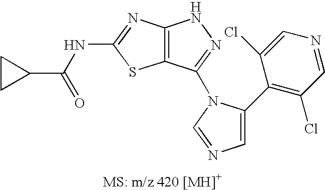

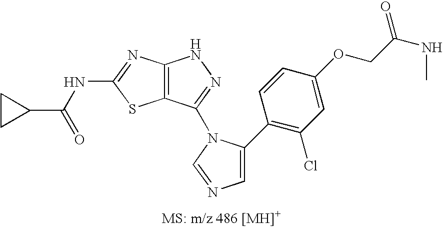

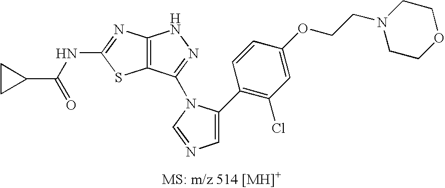

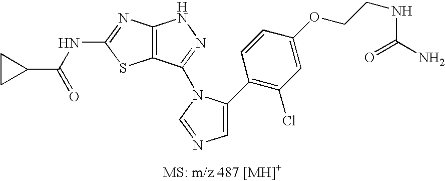

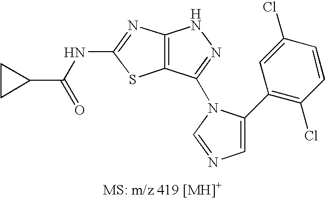

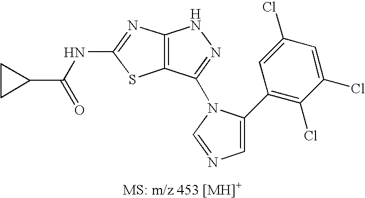

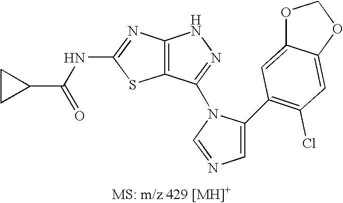

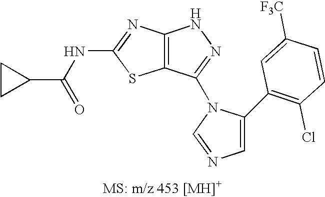

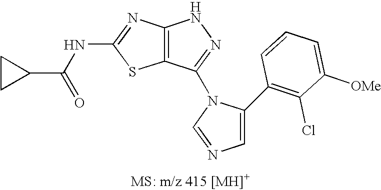

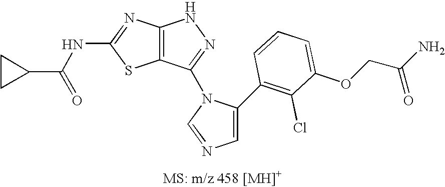

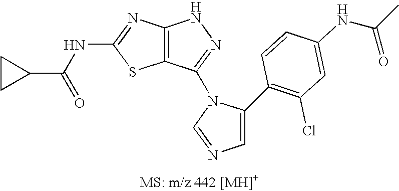

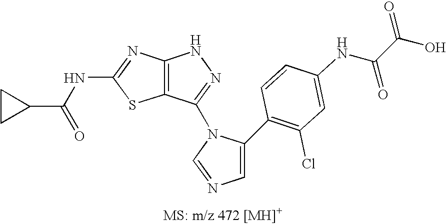

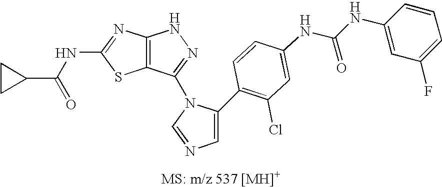

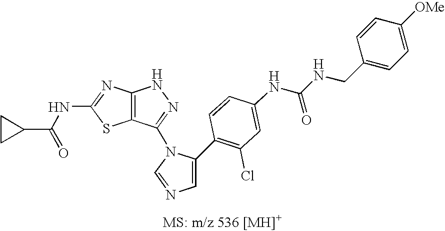

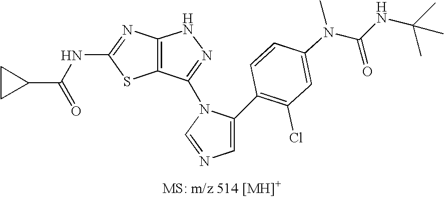

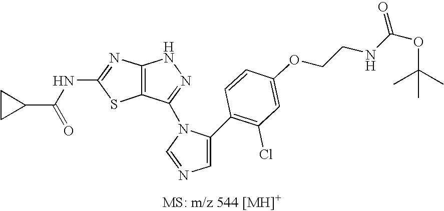

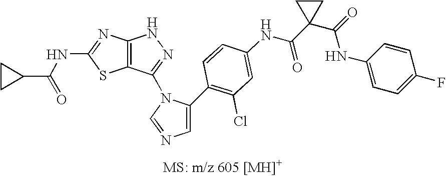

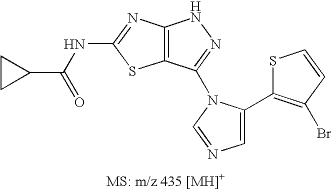

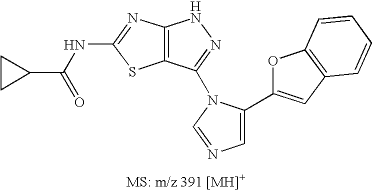

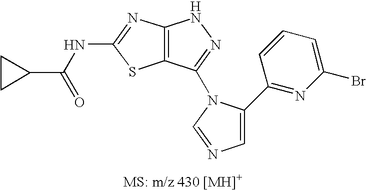

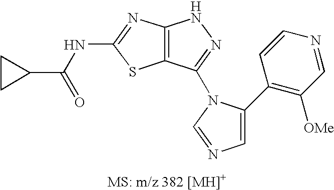

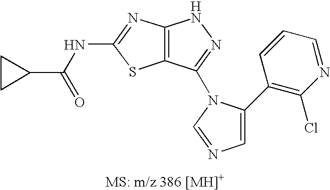

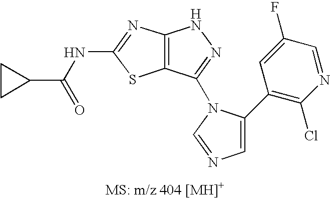

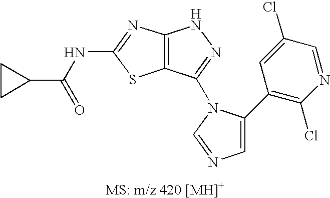

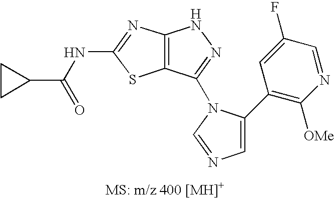

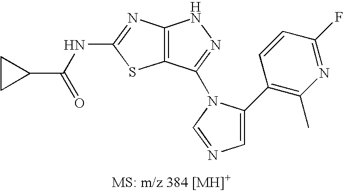

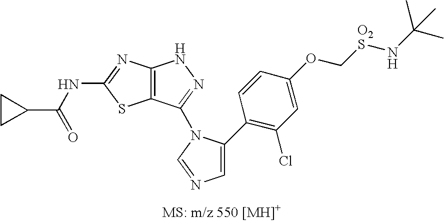

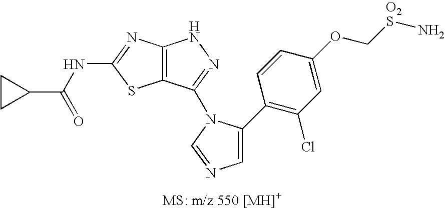

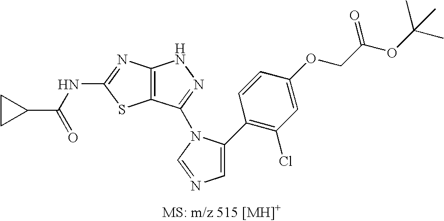

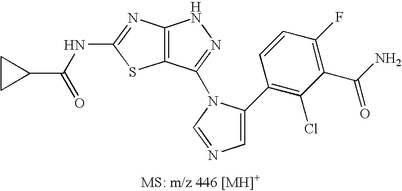

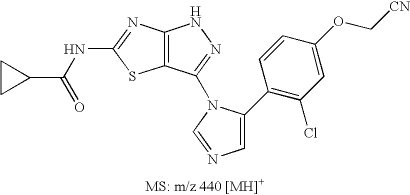

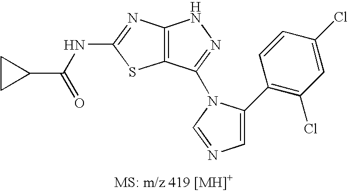

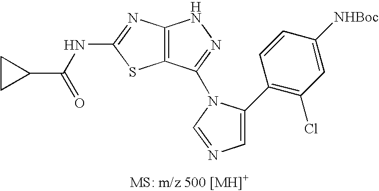

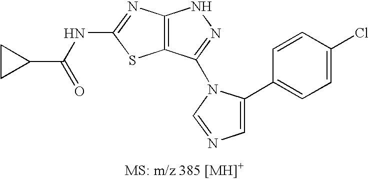

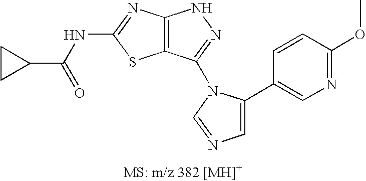

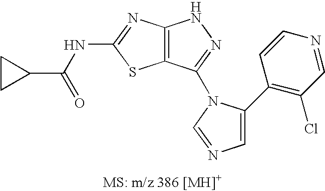

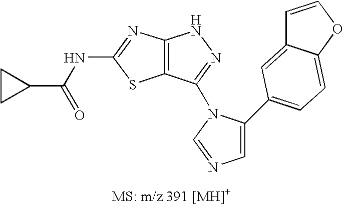

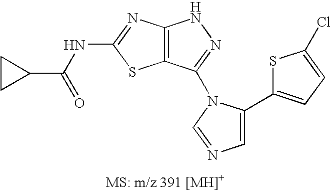

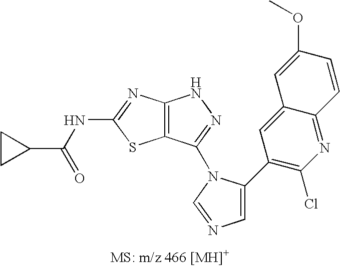

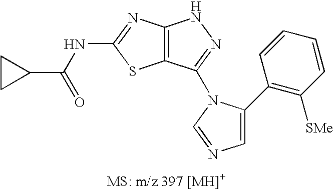

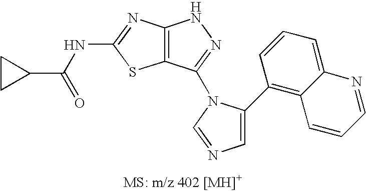

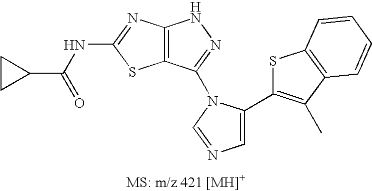

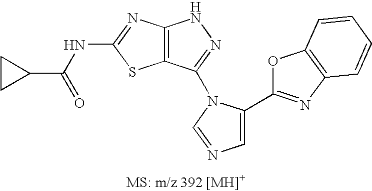

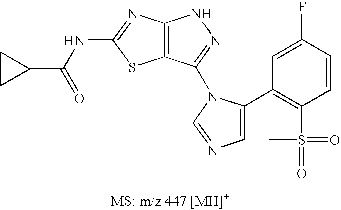

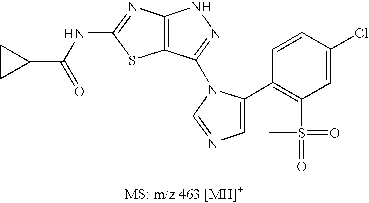

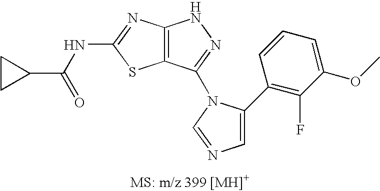

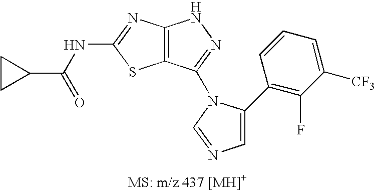

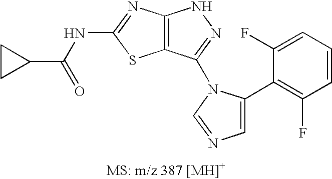

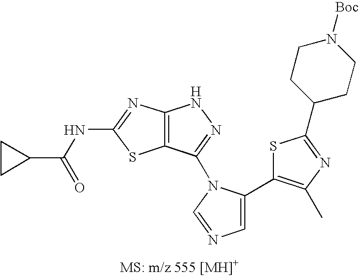

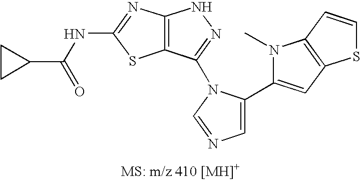

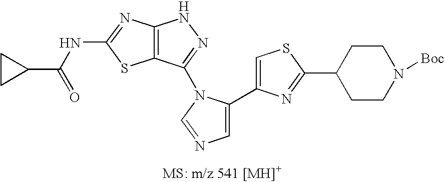

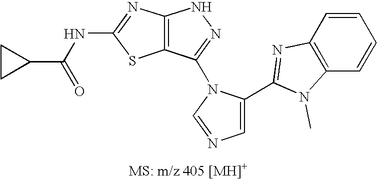

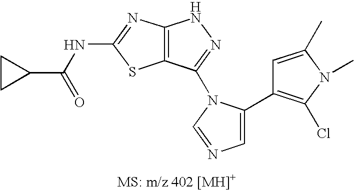

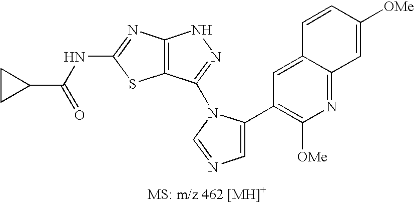

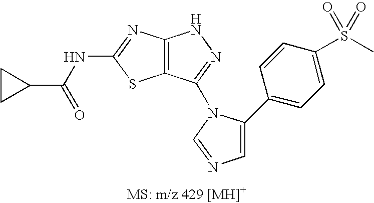

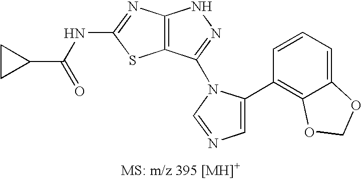

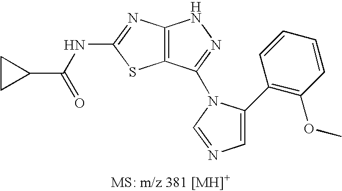

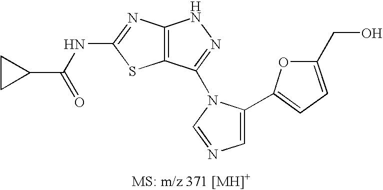

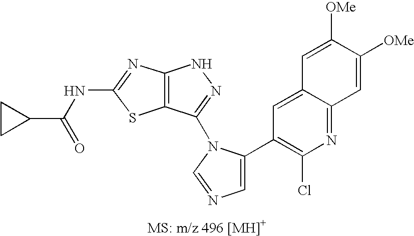

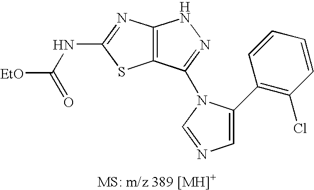

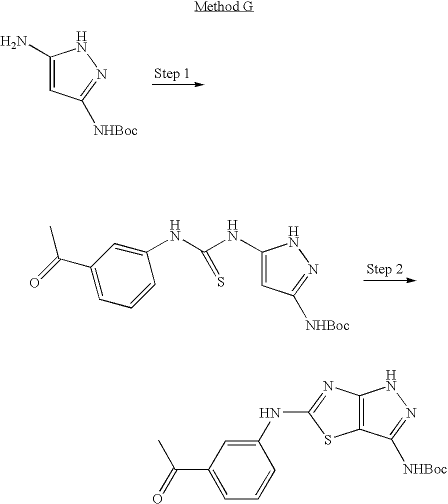

### Step 1: synthesis of {5-[3-(3-acetyl-phenyl)-thioureido]-1H-pyrazol-3-yl}-carbamic acid tert-butyl ester

To a solution of (5-amino-2H-pyrazol-3-yl)-carbamic acid tert-butyl ester (500 mg, 2.52 mmol) in THF (10 mL) was added 3-acetylphenyl isothiocyanate (448 mg, 2.52 mmol) in one portion. The reaction mixture was stirred at room temperature for 2 h, then directly adsorbed on silica gel. Purification on silica gel with 0-80% gradient of EtOAc/Hexanes as eluent provided 279 mg of {5-[3-(3-acetyl-phenyl)-thioureido]-1H-pyrazol-3-yl}-carbamic acid tert-butyl ester as a light yellow solid (30% yield): HPLC/MS m/z: 398 [MNa]⁺.

### Step 2: synthesis of [5-(3-acetyl-phenylamino)-1H-pyrazolo [3,4-d]thiazol-3-yl]-carbamic acid tert-butyl ester

To a solution of {5-[3-(3-acetyl-phenyl)-thioureido]-1H-pyrazol-3-yl}-carbamic acid tert-butyl ester (275 mg, 0.733 mmol) in AcOH (15 mL) was added a 1.5 M bromine solution in AcOH (0.49 mL, 0.733 mmol) dropwise. The reaction mixture was stirred at room temperature for 6 h, and then concentrated in vacuo. The crude was partitioned between saturated aqueous NaHCO₃ and EtOAc. The aqueous layer was extracted with EtOAc (3×), and the combined organic layers were adsorbed on silica gel. Purification on silica gel with 0-10% gradient of MeOH/CH₂Cl₂ as eluent provided 74 mg of [5-(3-acetyl-phenylamino)-1H-pyrazolo[3,4-d]thiazol-3-yl]-carbamic acid tert-butyl ester as an off white solid (27% yield): ¹H NMR (d₆-DMSO) δ 12.4 (broad s, 1H), 10.5 (broad s, 1H), 9.94 (broad s, 1H), 8.24 (s, 1H), 7.91 (d, 1H), 7.61 (d, 1H), 7.49 (t, 1H), 2.58 (s, 3H), 1.45 (s, 9H); HPLC/MS m/z: 374 [MH]⁺.

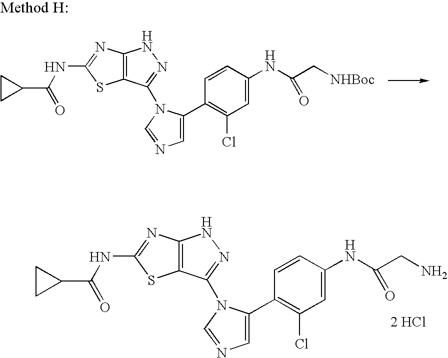

### Synthesis of cyclopropanecarboxylic acid (3-{5-[4-(2-amino-acetylamino)-2-chloro-phenyl]-imidazol-1-yl}-1H-pyrazolo [3,4-d]thiazol-5-yl)-amide, dihydrochloride salt

[(3-Chloro-4-{3-[5-(cyclopropanecarbonyl-amino)-1H-pyrazolo[3,4-d]thiazol-3-yl]-3H-imidazol-4-yl}-phenylcarbamoyl)-methyl]-carbamic acid tert-butyl ester (4.0 mg, 0.0072 mmol) [prepared according to method F] was treated with a 4 N solution of HCl in dioxane. The reaction mixture was stirred for 1 h, and the resulting precipitate was filtered, washed with EtOAc, and dried in vacuo to provide 2.5 mg of cyclopropanecarboxylic acid (3-{5-[4-(2-amino-acetylamino)-2-chloro-phenyl]-imidazol-1-yl}-1H-pyrazolo[3,4-d]thiazol-5-yl)-amide dihydrochloride salt as a yellow solid (66% yield). 1H NMR (d₆-DMSO) δ 12.6 (s, 1H), 10.9 (s, 1H), 9.05 (broad s, 1H), 8.02 (m, 4H), 7.68 (d, 1H), 7.58 (broad s, 1H), 7.40 (dd, 1H), 7.30 (d, 1H), 3.63 (q, 2H), 1.76 (m, 1H), 0.75 (m, 2H), 0.70 (m, 2H); HPLC/MS m/z: 457 [MH]⁺.

Other compounds prepared by method H:

TABLE 4

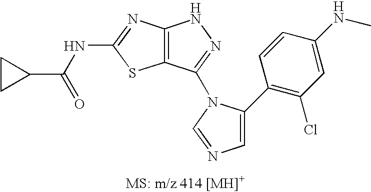

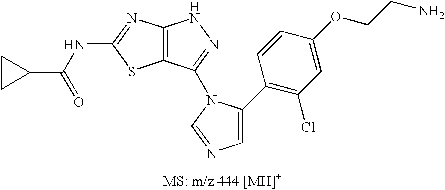

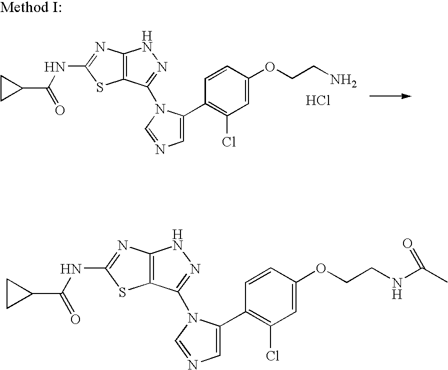

### Synthesis of cyclopropanecarboxylic acid (3-{5-[4-(2-acetylamino-ethoxy)-2-chloro-phenyl]-imidazol-1-yl}-1H-pyrazolo [3,4-d]thiazol-5-yl)-amide

To a solution of cyclopropanecarboxylic acid (3-{5-[4-(2-amino-ethoxy)-2-chloro-phenyl]-imidazol-1-yl}-1H-pyrazolo[3,4-d]thiazol-5-yl)-amide hydrochloride salt (10 mg, 0.021 mmol) and triethylamine (0.015 mL, 0.1 05 mmol) in DMF (0.4 mL) was added acetyl chloride (0.0016 mL, 0.022 mmol). The reaction mixture was stirred at room temperature for 2 h, then it was adsorbed on silica gel. Purification on silica gel with 0-10% gradient of MeOH/CH₂Cl₂ as eluent provided 5.0 mg of cyclopropanecarboxylic acid (3-{5-[4-(2-acetylamino-ethoxy)-2-chloro-phenyl]-imidazol-1-yl}-1H-pyrazolo [3,4-d]thiazol-5-yl)-amide as a white solid (49% yield). ¹H NMR (d₆-DMSO) δ 13.4 (broad s, 1H), 12.6 (broad s, 1H), 8.15 (s, 1H), 8.08 (t, 1H), 7.32 (d, 1H), 7.10 (m, 2H), 6.98 (d, 1H), 4.00 (t, 2H), 3.36 (q, 2H), 1.89 (m, 1H), 1.81 (s, 3H), 0.90 (m, 2H), 0.86 (m, 2H); HPLC/MS m/z: 486 [MH]⁺.

Other compounds prepared by method I:

TABLE 5

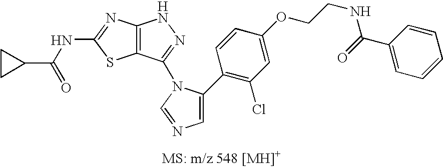

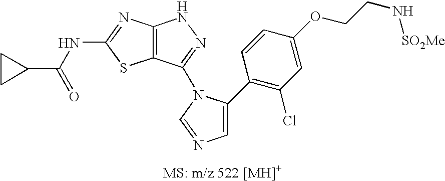

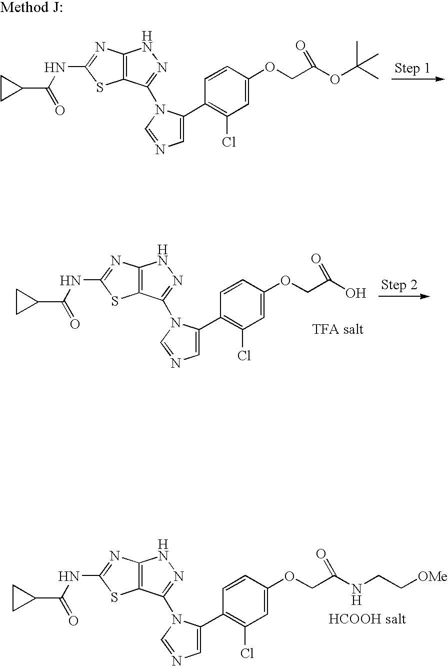

### Step 1: Synthesis of (3-chloro-4-{3-[5-(cyclopropanecarbonyl-amino)-1H-pyrazolo[3,4-d]thiazol-3-yl]-3H-imidazol-4-yl}-phenoxy)-acetic acid, trifluoroacetic acid salt

To a suspension of (3-chloro-4-{3-[5-(cyclopropanecarbonyl-amino)-1H-pyrazolo[3,4-d]thiazol-3-yl]-3H-imidazol-4-yl}-phenoxy)-acetic acid tert-butyl ester (18 mg, 0.035 mmol) and PS-thiophenol (48 mg, 0.07 mmol, Argonaut resin) in dichloromethane (0.5 mL) was added trifluoroacetic acid (0.5 mL). The reaction mixture was stirred at room temperature for 1 h. The resin was then filtered and washed with dichloromethane. The filtrate was concentrated in vacuo. The residue was triturated with diethyl ether, filtered, washed with diethyl ether, and dried in vacuo to provide 15 mg of (3-chloro-4-{3-[5-(cyclopropanecarbonyl-amino)-1H-pyrazolo [3,4-d]thiazol-3-yl]-3H-imidazol-4-yl}-phenoxy)-acetic acid, trifluoroacetic acid salt, as a white solid (75% yield). ¹H NMR (d₆-DMSO) δ 13.7 (broad s, 1H), 12.7 (s, 1H), 8.89 (broad s, 1H), 7.55 (broad s, 1H), 7.40 (d, 1H), 7.11 (d, 1H), 7.01 (dd, 1H), 4.73 (s, 2H), 1.91 (m, 1H), 0.92 (m, 2H), 0.89 (m, 2H); HPLC/MS m/z: 459 [MH]⁺.

### Step 2: Synthesis of cyclopropanecarboxylic acid [3-(5-{2-chloro-4-[(2-methoxy-ethylcarbamoyl)-methoxy]-phenyl}-imidazol-1-yl)-1H-pyrazolo[3,4-d]thiazol-5-yl]-amide, formic acid salt

A vial under nitrogen atmosphere was charged with (3-chloro-4-{3-[5-(cyclopropanecarbonyl-amino)-1H-pyrazolo[3,4-d]thiazol-3-yl]-3H-imidazol-4-yl}-phenoxy)-acetic acid, trifluoroacetic acid salt (20 mg, 0.035 mmol), sodium bicarbonate (8.8 mg, 0.105 mmol), 1-hydroxybenzotriazole (7 mg, 0.0525 mmol), and N-(3-dimethylaminopropyl)-N′-ethylcarbodiimide hydrochloride (10 mg, 0.0525 mmol). DMF (0.3 mL) was added, followed by 2-methoxyethylamine (0.0037 mL, 0.042 mmol). The reaction mixture was stirred at room temperature for 16 h. The crude mixture was diluted to 0.8 mL volume with DMSO and filtered through a 0.45 micron filter. The filtrate was directly purified by mass-triggered reverse-phase preparative HPLC (C 18 column) to provide 11.9 mg of cyclopropanecarboxylic acid [3-(5-{2-chloro-4-[(2-methoxy-ethylcarbamoyl)-methoxy]-phenyl}-imidazol-1-yl)-1H-pyrazolo[3,4-d]thiazol-5-yl]-amide, formic acid salt, as a white solid (61% yield). ¹H NMR (d₆-DMSO) δ 8.12 (s, 1H), 8.10 (s, 1H), 8.07 (t, 1H), 7.30 (d, 1H), 7.06 (m, 2H), 6.95 (dd, 1H), 4.45 (s, 2H), 3.29 (t, 2H), 3.23 (q, 2H), 3.16 (s, 3H), 1.84 (m, 1H), 0.85 (m, 2H), 0.81 (m, 2H); HPLC/MS m/z: 516 [MH]⁺.

Other compounds prepared by method J:

TABLE 6

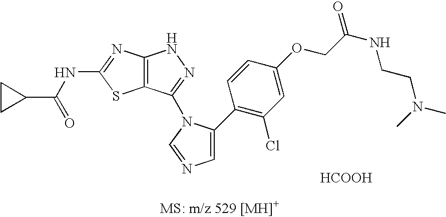

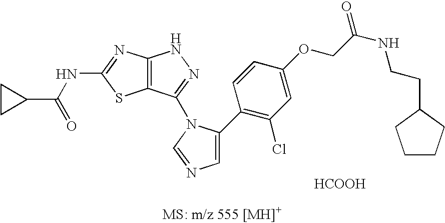

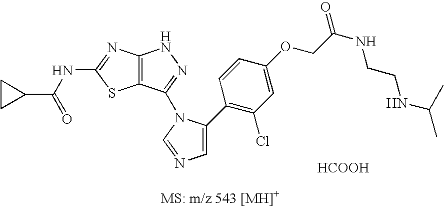

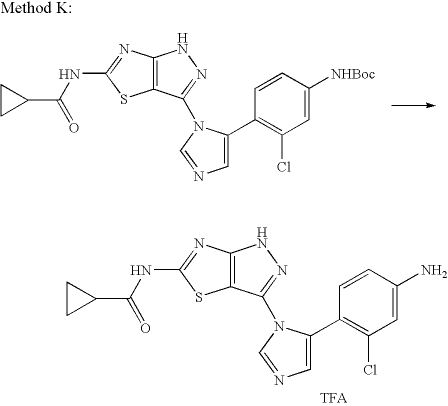

### Synthesis of cyclopropanecarboxylic acid {3-[5-(4-amino-2-chloro-phenyl)-imidazol-1-yl]-1H-pyrazolo[3,4-d]thiazol-5-yl}-amide, trifluoroacetic acid salt

To a vial charged with (3-Chloro-4-{3-[5-(cyclopropanecarbonyl-amino)-1H-pyrazolo[3,4-d]thiazol-3-yl]-3H-imidazol-4-yl}-phenyl)-carbamic acid tert-butyl ester (13.4 mg, 0.027 mmol) and PS-thiophenol (50 mg, 0.075 mmol, Argonaut resin) was added trifluoroacetic acid (1.5 mL). The reaction mixture was stirred at room temperature for 2 h, and then the resin was filtered and washed with MeOH. The filtrate was evaporated and dried in vacuo to provide 13.8 mg of cyclopropanecarboxylic acid {3-[5-(4-amino-2-chloro-phenyl)-imidazol-1-yl]-1H-pyrazolo[3,4-d]thiazol-5-yl}-amide, trifluoroacetic acid salt. ¹H NMR (d₆-DMSO) δ 13.9 (broad s, 1H), 12.6 (broad s, 1H), 9.45 (s, 1H), 7.80 (s, 1H), 7.1 (d, 1 H), 6.62 (d, 1H), 6.54 (dd, 1H), 3.9 (s, 2H), 1.9 (m, 1H), 0.9 (m, 4H); HPLC/MS m/z: 385 [MH]⁺.

Other compounds prepared by method K:

TABLE 7

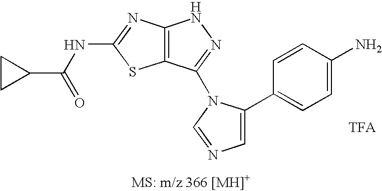

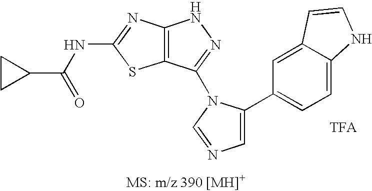

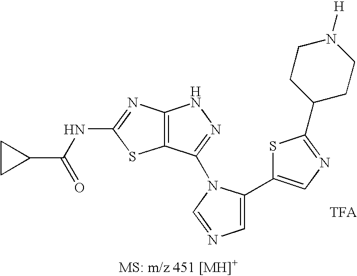

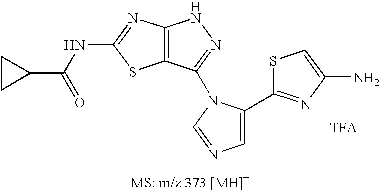

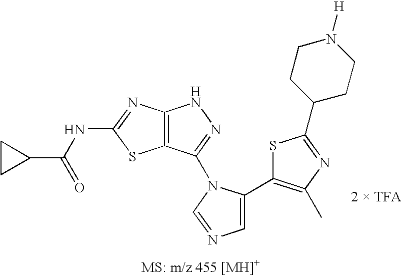

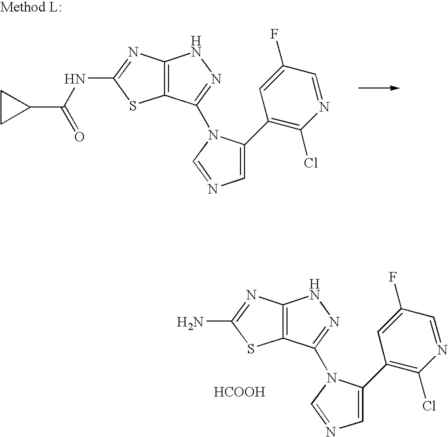

### Synthesis of 3-[5-(2-chloro-5-fluoro-pyridin-3-yl)-imidazol-1-yl]-1H-pyrazolo [3,4-d]thiazol-5-ylamine, formic acid salt

To a suspension of cyclopropanecarboxylic acid {3-[5-(2-chloro-5-fluoro-pyridin-3-yl)-imidazol-1-yl]-1H-pyrazolo[3,4-d]thiazol-5-yl}-amide (7.5 mg, 0.0186 mmol) in water (0.5 mL) in a microwave vessel was added 70% aqueous solution of perchloric acid (0.05 mL). The reaction was run a Personal Chemistry microwave reactor at 150° C. for 30 min. Crude material was directly purified by mass-triggered reverse-phase preparative HPLC (C18 column) to provide 2.3 mg of 3-[5-(2-chloro-5-fluoro-pyridin-3-yl)-imidazol-1-yl]-1H-pyrazolo[3,4-d]thiazol-5-ylamine, formic acid salt, as a white solid (32% yield). ¹H NMR (d₆-DMSO) δ 12.9 (s, 1H), 8.49 (d, 1H), 8.14 (s, 1H), 7.96 (dd, 1H), 7.61 (broad s, 2H), 7.23 (s, 1H), 6.47 (s, 1H); HPLC/MS m/z: 336 [MH]⁺.

Other compound prepared by method L:

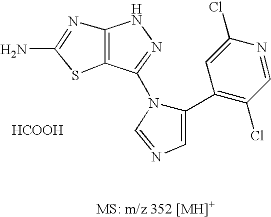

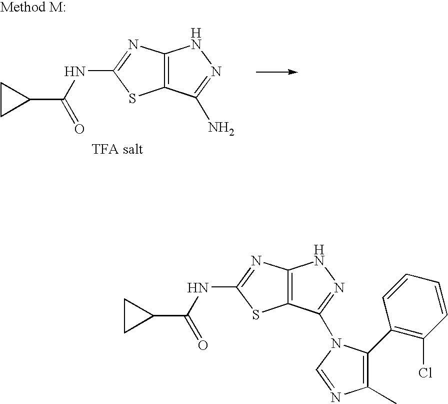

### Synthesis of cyclopropanecarboxylic acid {3-[5-(2-chloro-phenyl)-4-methyl-imidazol-1-yl]-1H-pyrazolo[3,4-d]thiazol-5-yl}-amide

To a suspension of cyclopropanecarboxylic acid (3-amino-1H-pyrazolo[3,4-d]thiazol-5-yl)-amide, trifluoroacetic acid salt (200 mg, 0.594 mmol) in absolute EtOH (5.0 mL) was added 2-chlorobenzaldehyde (0.08 mL, 0.712 mmol). The reaction mixture was refluxed for 16 h, and then it was dried in vacuo. The crude solid was dissolved in DMF (5.0 mL) under nitrogen atmosphere. Potassium carbonate (246 mg, 1.782 mmol) was added, followed by 1-methyl-1-tosylmethyl isocyanide (124 mg, 0.594 mmol). The reaction mixture was stirred at 80° C. for 3 h, and then concentrated in vacuo. The crude solid was redissolved in 10% MeOH/CH₂Cl₂ and adsorbed on silica gel. Purification on silica gel with 0-8% gradient of MeOH/CH₂Cl₂ as eluent provided 54 mg of cyclopropanecarboxylic acid {3-[5-(2-chloro-phenyl)-4-methyl-imidazol-1-yl]-1H-pyrazolo [3,4-d]thiazol-5-yl }-amide as a cream-colored solid (23% yield). ¹H NMR (d₆-DMSO) δ 13.4 (broad s, 1H), 12.6 (broad s, 1H), 8.06 (s, 1H), 7.5 (d, 1H), 7.36-7.44 (m, 3 H), 2.05 (s, 3H), 1.9 (m, 1H), 0.9 (m, 4H); HPLC/MS m/z: 399 [MH]⁺.

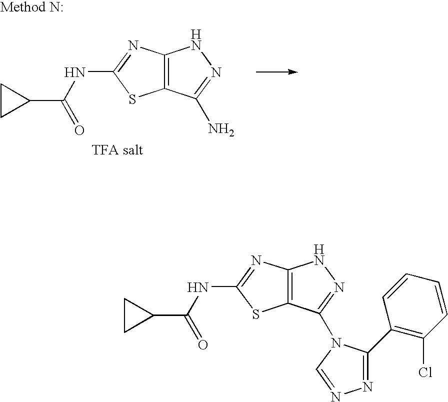

### Synthesis of cyclopropanecarboxylic acid {3-[3-(2-chloro-phenyl)-[1,2,4]triazol-4-yl]-1H-pyrazolo[3,4-d]thiazol-5-yl}-amide

To cyclopropanecarboxylic acid (3-amino-1H-pyrazolo[3,4-d]thiazol-5-yl)-amide, trifluoroacetic acid salt (100 mg, 0.297 mmol) was added 2-(2-chloro-phenyl)-[1,3,4]oxadiazole (350 mg, 1.93 mmol) neat [oxadiazole preparation: Bulletin de la Societe Chimique de France (1962), 1580-91]. The mixture was stirred at 120° C. for 2 h. The crude mixture was dissolved in 10% MeOH/CH₂Cl₂ and adsorbed on silica gel. Purification on silica gel with 0-9% gradient of MeOH/CH₂Cl₂ as eluent provided 16 mg of cyclopropanecarboxylic acid {3-[3-(2-chloro-phenyl)-[1,2,4]triazol-4-yl]-1H-pyrazolo[3,4-d]thiazol-5-yl}-amide (14% yield). ¹H NMR (d₆-DMSO) δ 13.7 (broad s, 1H), 12.6 (broad s, 1H), 8.80 (s, 1H), 7.66 (dd, 1H), 7.52-7.62 (m, 3H), 1.91 (m, 1H), 0.88-0.93 (m, 4H); HPLC/MS m/z: 386 [MH]⁺.

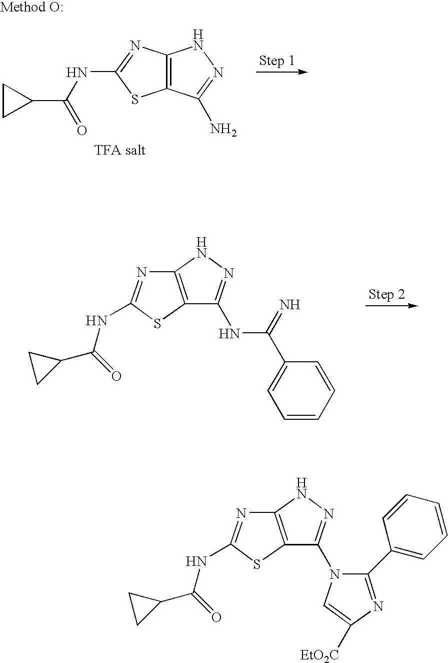

### Step 1: Synthesis of cyclopropanecarboxylic acid [3-(benzimidoyl-amino)-1H-pyrazolo [3,4-d]thiazol-5-yl]-amide

To cyclopropanecarboxylic acid (3-amino-1H-pyrazolo[3,4-d]thiazol-5-yl)-amide, trifluoroacetic acid salt (500 mg, 1.48 mmol) was added acetonitrile (2.0 mL) followed by triethylamine (0.227 mL, 1.63 mmol). The reaction was stirred for 5 min and then methylbenzimidate (0.508 g, 2.96 mmol) was added. The mixture was heated at 55° C. overnight. The precipitate was then filtered and washed with acetonitrile to provide 0.37 g of cyclopropanecarboxylic acid [3-(benzimidoyl-amino)-1H-pyrazolo [3,4-d]thiazol-5-yl]-amide (77% yield), which was used directly in the next step.

### Step 2: Synthesis of 1-[5-(cyclopropanecarbonyl-amino)-1H-pyrazolo[3,4-d]thiazol-3-yl]-2-phenyl-1H-imidazole-4-carboxylic acid ethyl ester

To cyclopropanecarboxylic acid [3-(benzimidoyl-amino)-1H-pyrazolo[3,4-d]thiazol-5-yl]-amide (100 mg, 0.31 mmol) and NaHCO₃ (50 mg, 0.6 mmol) was added 2-propanol (7.5 mL). The mixture was heated to 40° C. and then ethyl bromopyruvate (53 uL, 0.42 mmol) was added dropwise. The mixture was heated at 80° C. for 2 days. Purification on silica gel with 0-9% gradient of MeOH/CH₂Cl₂ as eluent provided 15 mg of 1-[5-(cyclopropanecarbonyl-amino)-1H-pyrazolo [3,4-d]thiazol-3-yl]-2-phenyl-1H-imidazole-4-carboxylic acid ethyl ester (12% yield). ¹H NMR (d₆-DMSO) δ 13.7 (broad s, 1H), 12.7 (broad s, 1H), 8.2 (s, 1H), 7.2 (m, 5H), 4.3 (q, 2H), 1.91 (m, 1H), 1.3 (t, 3H), 0.9 (m, 4H); HPLC/MS m/z: 423 [MH]⁺.

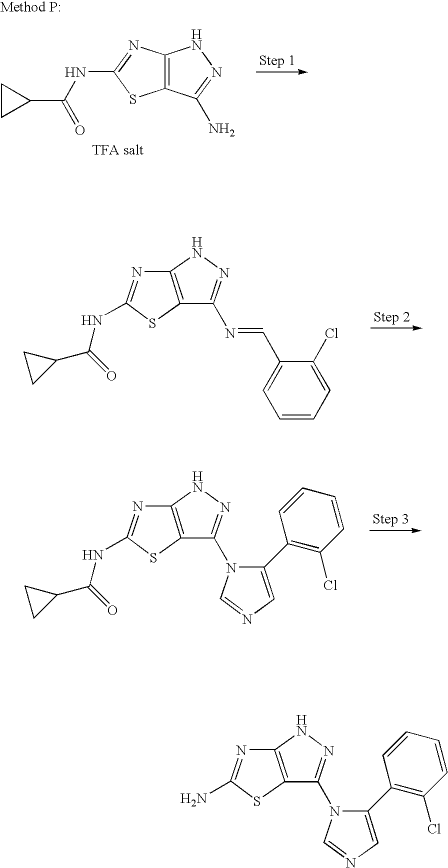

### Step 1: Synthesis of cyclopropanecarboxylic acid {3-[(2-chloro-benzylidene)-amino]-1H-pyrazolo [3,4-d]thiazol-5-yl}-amide

To a solution of cyclopropanecarboxylic acid (3-amino-1H-pyrazolo[3,4-d]thiazol-5-yl)-amide, trifluoroacetic acid salt (2.10 g, 6.23 mmol) in absolute EtOH (40 mL) was added 2-chlorobenzaldehyde (841 uL, 7.45 mmol). The reaction mixture was heated at 80° C. in an oil bath for 18 h under a reflux condenser and N₂ inlet. The ethanol was removed via rotary evaporation. Fresh ethanol was added and removed via rotary evaporation (3 cycles). No purification was necessary to provide 2.53 g of pure cyclopropanecarboxylic acid {3-[(2-chloro-benzylidene)-amino]-1H-pyrazolo[3,4-d]thiazol-5-yl}-amide as a yellow solid (quantitative yield). ¹H NMR (d₆-DMSO) δ 12.8 (s, 1H), 8.92 (broad s, 1H), 8.21 (d, 1H), 7.64 (d, 1H), 7.57 (m, 4H), 2.00 (m, 1H), 0.96 (m, 4H); HPLC/MS m/z: 364 [MH]⁺.

### Step 2: Synthesis of cyclopropanecarboxylic acid {3-[5-(2-chloro-phenyl)-imidazol-1-yl]-1H-pyrazolo [3,4-d]thiazol-5-yl}-amide

A solution of cyclopropanecarboxylic acid {3-[(2-chloro-benzylidene)-amino]-1H-pyrazolo[3,4-d]thiazol-5-yl}-amide (2.15 g, 6.21 mmol), potassium carbonate (2.58 g, 18.7 mmol), and tosylmethyl isocyanide (1.33 g, 6.83 mmol) in DMF (60 mL) was stirred at ambient temperature for 1.5 h. The reaction mixture was then heated in an oil bath at 80° C. for 18 h under a reflux condenser and N₂ inlet. After the reaction had cooled to room temperature, 1 M aqueous citric acid was added until pH=5-6. The compound was extracted into EtOAc and washed with H₂O (3×). The organic layer was dried over sodium sulfate, filtered, and concentrated in vacuo. The material was redissolved in EtOAc and adsorbed onto silica gel. Purification in a gradient of 0-100% 1:10 MeOH:EtOAc and Hexanes afforded 1.66 g of cyclopropanecarboxylic acid {3-[5-(2-chloro-phenyl)-imidazol-1-yl]-1H-pyrazolo[3,4-d]thiazol-5-yl}-amide as a peach powder (69% yield). ¹H NMR (d₄MeOH) δ 8.22 (d, 1H), 7.43 (m, 4H), 7.20 (d, 1H), 1.82 (m, 1H), 0.98 (m, 4H); HPLC/MS m/z: 385 [MH]⁺.

### Step 3: Synthesis of 3-[5-(2-chloro-phenyl)-imidazol-1-yl]-1H-pyrazolo[3,4-d]thiazol-5-ylamine

To a solution of cyclopropanecarboxylic acid {3-[5-(2-chloro-phenyl)-imidazol-1-yl]-1H-pyrazolo[3,4-d]thiazol-5-yl}-amide (4.17 g, 10.8 mmol) in a mixture of H₂O (40 mL) and EtOH (160 mL) was added a 70% aqueous solution of perchloric acid (40 mL). The reaction mixture was heated in an oil bath at 105° C. under a reflux condenser and N₂ inlet for 22 h. The EtOH was removed by rotary evaporation and then sodium bicarbonate was added until pH=6-7. The product was extracted into EtOAc and washed with H₂O and brine. The organic layer was dried over sodium sulfate, filtered, and concentrated to an orange solid. The solid was triturated with methylene chloride and the precipitate was filtered through a fritted filter. The collected precipitate was washed with ether to afford 3-[5-(2-chloro-phenyl)-imidazol-1-yl]-1H-pyrazolo[3,4-d]thiazol-5-ylamine as a tan powder (92% yield). ¹H NMR (d₄-MeOH) δ 8.17 (s, 1H), 7.43 (m, 4H), 7.18 (s, 1H); HPLC/MS m/z: 317 [MH]⁺.

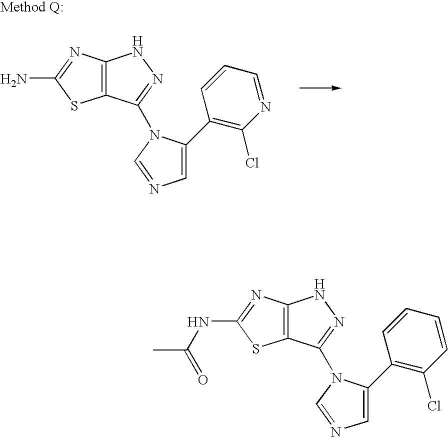

### Synthesis of N-{3-[5-(2-chloro-phenyl)-imidazol-1-yl]-1H-pyrazolo [3,4-d]thiazol-5-yl}-acetamide

To a solution of 3-[5-(2-chloro-phenyl)-imidazol-1-yl]-1H-pyrazolo[3,4-d]thiazol-5-ylamine (25 mg, 0.079 mmol) and pyridine (38 uL, 0.474 mmol) in THF (1 mL) was added acetyl chloride (28 uL, 0.395 mmol). The reaction mixture was heated at 80° C. for 15 h. To the reaction was added N,N-dimethylethylenediamine (60 uL, 0.553 mmol) and the reaction was stirred at ambient temperature for 16 h. The product was extracted into EtOAc and washed with 1 M aqueous citric acid. The organic layer was dried over sodium sulfate, filtered, and adsorbed onto silica gel. Purification with a gradient of 0-100% EtOAc/Hexanes as eluent provided 2.9 mg of N-{3-[5-(2-chloro-phenyl)-imidazol-1-yl]-1H-pyrazolo[3,4-d]thiazol-5-yl}-acetamide as a white powder (10% yield). ¹H NMR (d₄-MeOH) δ 8.23 (s, 1H), 7.45 (m, 4H), 7.21 (s, 1H), 2.16 (s, 3H); HPLC/MS m/z: 359 [MH]⁺.

Other compounds prepared by method Q:

TABLE 8

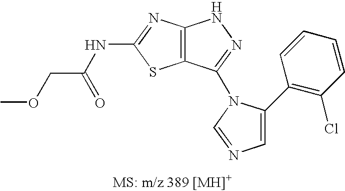

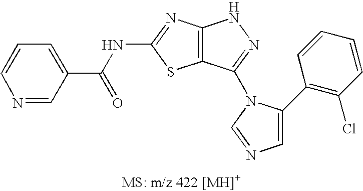

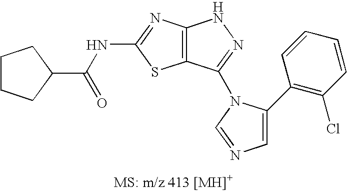

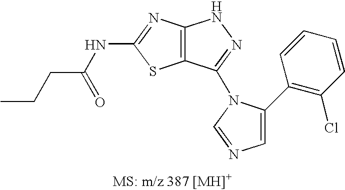

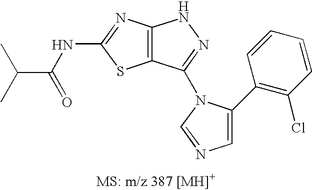

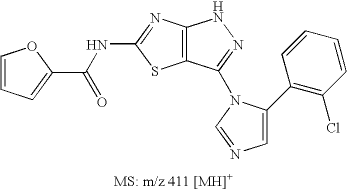

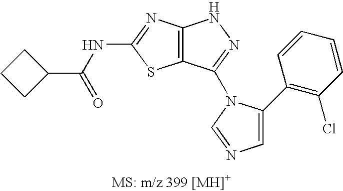

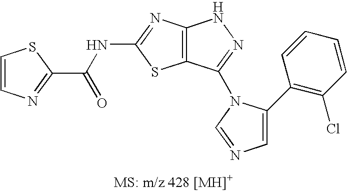

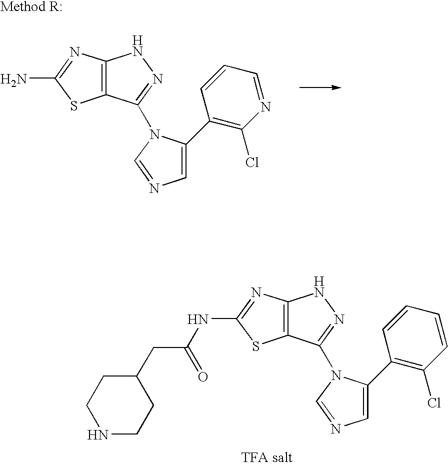

### Synthesis of N-{3-[5-(2-chloro-phenyl)-imidazol-1-yl]-1H-pyrazolo [3,4-d]thiazol-5-yl}-2-piperidin-4-yl-acetamide, trifluoroacetic acid salt

In a microwave vial was combined 3-[5-(2-chloro-phenyl)-imidazol-1-yl]-1H-pyrazolo[3,4-d]thiazol-5-ylamine (27 mg, 0.085 mmol), 1-boc-4-piperidylacetic acid (62 mg, 0.256 mmol), HATU (97 mg, 0.256 mmol), and diisopropylethylamine (30 uL, 0.170 mmol) in DMF (1 mL). The microwave vial was sealed and heated in a Personal Chemistry microwave reactor at 90° C. for 900 seconds. After the heating was complete, N,N-dimethylethylenediamine (37 uL, 0.340 mmol) was added and the reaction was stirred at ambient temperature for 16 h. The Boc-protected intermediate was extracted into EtOAc and washed with a saturated aqueous solution of sodium bicarbonate and 1 M aqueous citric acid. The organic layer was dried over sodium sulfate, filtered, and adsorbed onto silica gel. Purification in a gradient of 0-100% 1:10 MeOH:EtOAc and Hexanes afforded 73 mg of 4-({3-[5-(2-chloro-phenyl)-imidazol-1-yl]-1H-pyrazolo[3,4-d]thiazol-5-ylcarbamoyl}-methyl)-piperidine-1-carboxylic acid tert-butyl ester as a white film (quantitative yield). To a solution of the intermediate dissolved in 5 mL dichloromethane was added PS-thiophenol resin (277 mg, 0.406 mmol, 1.46 mmol/g load capacity, Argonaut resin) and trifluoroacetic acid (5 mL). The reaction mixture was shaken gently at ambient temperature for 2.5 h. The resin was filtered off and rinsed with dichloromethane, MeOH, and diethyl ether. The filtrate was concentrated and the resulting solid was triturated with diethyl ether. The ether was decanted to afford 20.3 mg of N-{3-[5-(2-chloro-phenyl)-imidazol-1-yl]-1H-pyrazolo[3,4-d]thiazol-5-yl}-2-piperidin-4-yl-acetamide, trifluoroacetic acid salt as a light peach powder (43% yield). ¹H NMR (d₄-MeOH) δ 9.18 (s, 1H), 7.71 (s, 1H), 7.59 (d, 1H), 7.51 (m, 3H), 3.39 (m, 2H), 3.05 (m, 2H), 2.48 (d, 2H), 2.17 (m, 1H), 2.00 (m, 2H), 1.50 (q, 2H); HPLC/MS m/z: 442 [MH]⁺.

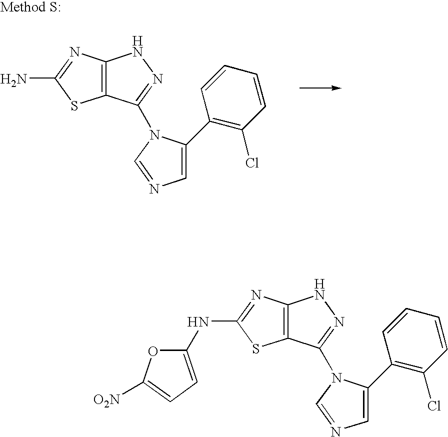

### Synthesis of {3-[5-(2-chloro-phenyl)-imidazol-1-yl]-1H-pyrazolo[3,4-d]thiazol-5-yl}-(5-nitro-furan-2-yl)-amine

To a solution of 3-[5-(2-chloro-phenyl)-imidazol-1-yl]-1H-pyrazolo[3,4-d]thiazol-5-ylamine (30 mg, 0.095 mmol) and 2-bromo-5-nitrofuran (27 mg, 0.142 mmol) in anhydrous DMSO (1 mL) was added NaH (4.5 mg, 0.190 mmol). The reaction mixture was stirred at ambient temperature for 16 h. The reaction mixture was diluted with 1 mL DMSO, filtered through a 0.45 um syringe filter, and purified by mass-triggered reverse phase chromatography in a mobile phase of H₂O and acetonitrile (with 0.1% formic acid as the modifier). Clean fractions were combined and lyophilized, affording 2.3 mg of {3-[5-(2-chloro-phenyl)-imidazol-1-yl]-1H-pyrazolo [3,4-d]thiazol-5-yl}-(5-nitro-furan-2-yl)-amine as a fluffy bright yellow powder (6% yield). ¹H NMR (d₆-DMSO) δ12.4 (broad s, 1H), 8.65 (broad s, 1H), 8.13 (s, 1H), 7.60 (broad s, 1H), 7.59 (d, 1H), 7.43 (d, 1H), 7.33 (t, 1H), 7.28 (t, 1H), 7.24 (d, 1H), 6.28 (broad s, 1H); HPLC/MS m/z: 428 [MH]⁺.

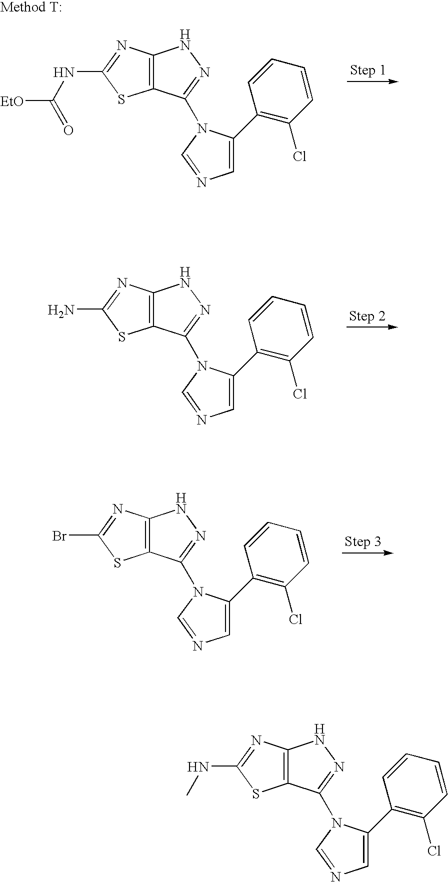

### Step 1: Synthesis of 3-[5-(2-Chloro-phenyl)-imidazol-1-yl]-1H-pyrazolo [3,4-d]thiazol-5-ylamine

To a Personal Chemistry 5 mL microwave vial was added {3-[5-(2-chloro-phenyl)-imidazol-1-yl]-1H-pyrazolo[3,4-d]thiazol-5-yl}-carbamic acid ethyl ester (106 mg, 0.27 mmol), EtOH (1 mL), water (1 mL) and aqueous solution of perchloric acid (600 uL, 40% w/w). The solution was heated in the microwave for 1 h at 150° C., then concentrated in vacuo. Purification by mass-triggered reverse-phase HPLC (C-18; gradient 5-95% ACN (0.1% formic acid): 0.1% formic acid in water) provided 11.9 mg of3-[5-(2-chloro-phenyl)-imidazol-1-yl]-1H-pyrazolo[3,4-d]thiazol-5-ylamine as a tan powder (14% yield). ¹H NMR (d₆-DMSO) δ 12.86 (broad s, 1H), 8.12 (s, 1H), 7.37 (m, 4H), 7.12 (s, 1H); HPLC/MS m/z: 317 [MH]⁺.

### Step 2: Synthesis of 5-Bromo-3-[5-(2-chloro-phenyl)-imidazol-1-yl]-1H-pyrazolo[3,4-d]thiazole

To an ice cold solution of CuBr₂ (533 mg, 2.38 mmol) in acetonitrile (8 mL) was added dropwise isoamyl nitrite (320 uL, 2.39 mmol). The solution was stirred for 5 min at 0° C. then an ice cold solution of 3-[5-(2-chloro-phenyl)-imidazol-1-yl]-1H-pyrazolo [3,4-d]thiazol-5-ylamine (518 mg, 1.63 mmol) in DMF (8 mL) was added dropwise over 5 min. The solution was allowed to warm up to room temperature, then it was heated to 60° C. for 2 h. The crude reaction mixture was concentrated, then partitioned between EtOAc and water. The organic phase was treated with brine, dried (NaSO₄), filtered and concentrated to obtain 147 mg of a green powder. Purification by flash column on silica gel eluting with a gradient of hexanes: EtOAc provided 6.2 mg of 5-bromo-3-[5-(2-chloro-phenyl)-imidazol-1-yl]-1H-pyrazolo[3,4-d]thiazole as a yellow powder (0.9% yield). ¹H NMR (d₆-DMSO) δ 12.02 (broad s, 1H), 8.27 (d 1H), 7.50 (m, 4H), 7.20 (s, 1H); HPLC/MS m/z: 379.9/381.9 [MH]⁺.

### Step 3: Synthesis of {3-[5-(2-chloro-phenyl)-imidazol-1-yl]-1H-pyrazolo[3,4-d]thiazol-5-yl}-methyl-amine

To a solution of 5-bromo-3-[5-(2-chloro-phenyl)-imidazol-1-yl]-1H-pyrazolo[3,4-d]thiazole (18 mg, 0.047 mmol) in THF (1 mL) was added 200 uL of a 40 wt % methylamine in aqueous solution. The reaction mixture was heated in an oil bath at 50° C. for 3 h. The starting material was still observed by LC/MS so an additional 200 uL of a 40 wt % methylamine in aqueous solution was added and the reaction was heated for another 16 h at 50° C. The reaction was concentrated in vacuo and then redissolved in 1 mL DMSO, filtered through a 0.45 um syringe filter, and purified by mass-triggered reverse phase chromatography in a mobile phase of H₂O and acetonitrile (with 0.1% formic acid as the modifier). Clean fractions were combined and lyophilized, affording 1.8 mg of {3-[5-(2-chloro-phenyl)-imidazol-1-yl]-1H-pyrazolo [3,4-d]thiazol-5-yl}-methyl-amine as a fluffy white powder (12% yield). ¹H NMR (d₆-DMSO) δ 12.9 (broad s, 1H), 8.06 (s, 1H), 7.93 (q, 1H), 7.46 (d, 1H), 7.37 (m, 1H), 7.33 (d, 2H), 7.08 (s, 1H), 2.72 (d, 3H); HPLC/MS m/z: 331 [MH]⁺.

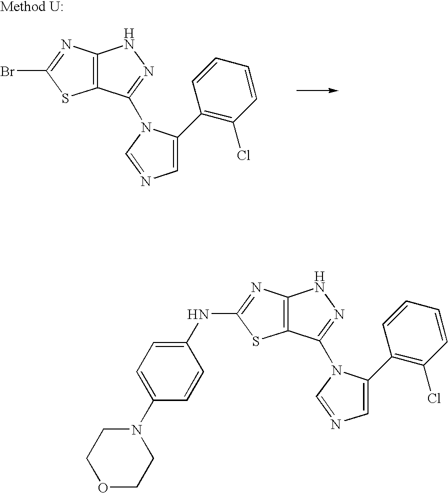

### Synthesis of {3-[5-(2-chloro-phenyl)-imidazol-1-yl]-1H-pyrazolo[3,4-d]thiazol-5-yl}-(4-morpholin-4-yl-phenyl)-amine

To a microwave vial was added 5-bromo-3-[5-(2-chloro-phenyl)-imidazol-1-yl]-1H-pyrazolo[3,4-d]thiazole (19 mg, 0.051 mmol), 4-morpholinoaniline (36 mg, 0.203 mmol), and anhydrous DMSO (1.25 mL). The microwave vial was sealed and heated in a Personal Chemistry microwave reactor at 150° C. for 1800 seconds. The crude reaction mixture was filtered through a 0.45 um syringe filter and purified by mass-triggered reverse phase chromatography in a mobile phase of H₂O and acetonitrile (with 0.1% formic acid as the modifier). Clean fractions were combined and lyophilized, affording 6.4 mg of {3-[5-(2-chloro-phenyl)-imidazol-1-yl]-1H-pyrazolo[3,4-d]thiazol-5-yl}-(4-morpholin-4-yl-phenyl)-amine as a fluffy purple powder (27% yield): ¹H NMR (d₆-DMSO) δ 13.1 (broad s, 1H), 10.2 (s, 1H), 8.11 (s, 1H), 7.48 (d, 1H), 7.36 (m, 5H), 7.11 (s, 1H), 6.87 (d, 2H), 3.66 (t, 4H), 2.98 (t, 4H); HPLC/MS m/z: 478 [MH]⁺.

Other compounds prepared by method U:

TABLE 9

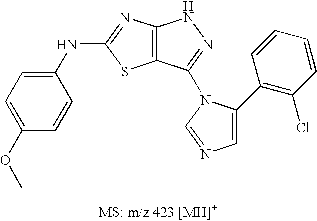

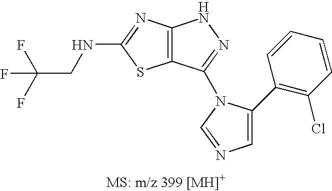

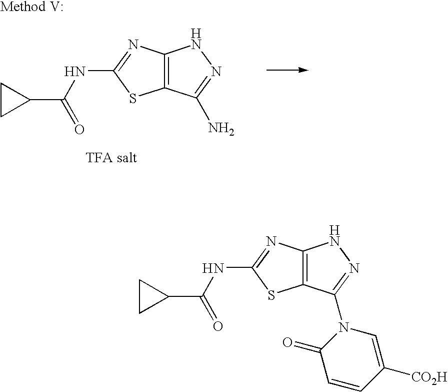

### Synthesis of 1-[5-(cyclopropanecarbonyl-amino)-1H-pyrazolo [3,4-d]thiazol-3-yl]-6-oxo-1,6-dihydro-pyridine-3-carboxylic acid

To a solution of cyclopropanecarboxylic acid (3-amino-1H-pyrazolo[3,4-d]thiazol-5-yl)-amide, trifluoroacetic acid salt (2.65 g, 7.86 mmol) in pyridine (50 mL) was added coumalic acid. The reaction mixture was stirred at room temperature for 8 h, and then concentrated in vacuo. The crude residue was treated with 1 M aqueous HCl, and the resulting precipitate was collected, washed with water, then Et₂O to provide 2.23 g of 1-[5-(cyclopropanecarbonyl-amino)-1H-pyrazolo[3,4-d]thiazol-3-yl]-6-oxo-1,6-dihydro-pyridine-3-carboxylic acid as a tan powder (82% yield). ¹H NMR (d₆-DMSO) δ 13.6 (broad s, 1H), 12.6 (s, 1H), 9.02 (d, 1H), 7.87 (dd, 1H), 6.62 (d, 1H), 1.97 (m, 1H), 1.93 (m, 1H), 0.89 (m, 4H); HPLC/MS m/z: 346 [MH]⁺.

Other compound prepared by method V:

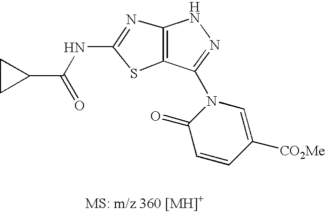

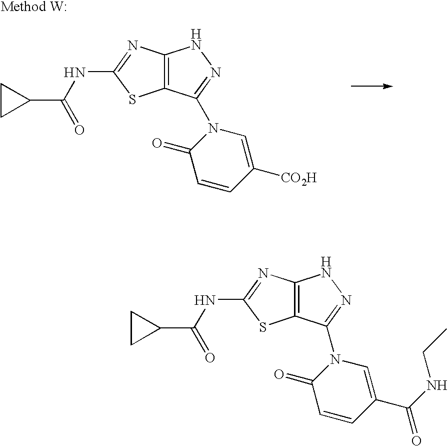

### Synthesis of 1-[5-(cyclopropanecarbonyl-amino)-1H-pyrazolo [3,4-d]thiazol-3-yl]-6-oxo-1,6-dihydro-pyridine-3-carboxylic acid ethylamide

To a solution of 1-[5-(cyclopropanecarbonyl-amino)-1H-pyrazolo[3,4-d]thiazol-3-yl]-6-oxo-1,6-dihydro-pyridine-3-carboxylic acid (50 mg, 0.145 mmol) in DMF (1 mL) was added HATU (80.9 mg, 0.213 mmol), diisopropylethylamine (40 uL, 0.229 mmol), and ethylamine (500 uL, 2 M in THF). The reaction mixture was heated to 90° C. in a Personal Chemistry microwave reactor for 15 min. The crude reaction mixture was diluted with EtOAc, washed with water and then brine. The organic phase was dried (NaSO₄), filtered and concentrated. Purification by flash column on silica gel eluting with a gradient of hexanes and 10% MeOH/EtOAc provided 1.8 mg of 1-[5-(cyclopropanecarbonyl-amino)-1H-pyrazolo[3,4-d]thiazol-3-yl]-6-oxo-1,6-dihydro-pyridine-3-carboxylic acid ethylamide as an tan powder (2% yield). ¹H NMR (d₆-DMSO) δ 13.5 (broad s, 1H), 12.5 (broad s, 1H), 8.88 (d, 1H), 8.45 (t, 1H), 7.89 (dd, 1H), 6.53 (d, 1H), 3.19(m, 2H), 1.92 (m, 1H), 1.93 (m, 1H), 1.04 (t, 3H), 0.87 (m, 4H); HPLC/MS m/z: 373 [MH]⁺.

Other compounds prepared by method W:

TABLE 10

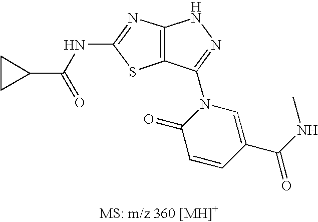

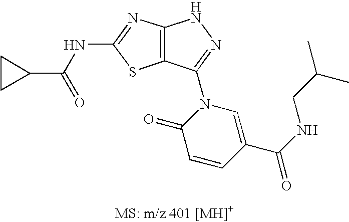

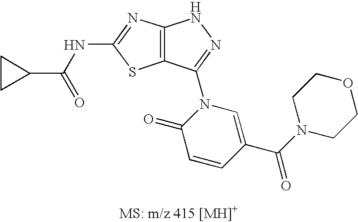

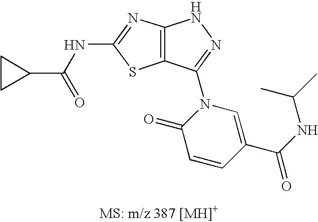

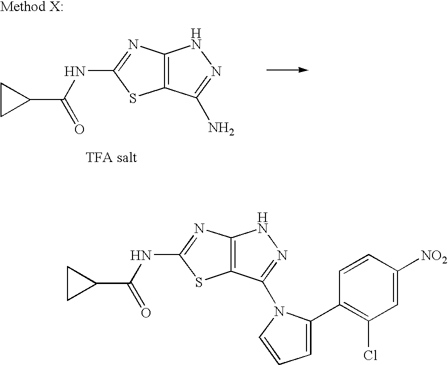

### Synthesis of cyclopropanecarboxylic acid {3-[2-(2-chloro-4-nitro-phenyl)-pyrrol-1-yl]-1H-pyrazolo[3,4-d]thiazol-5-yl}-amide

A solution of 3-amino-1H-pyrazolo[3,4-d]thiazol-5-yl)-amide, trifluoroacetic acid salt (100 mg, 0.29 mmol) and 1-(2-chloro-4-nitro-phenyl)-3-[1,3]dioxan-2-yl-propan-1-one (90 mg, 0.30 mmol) in HOAc (2 mL) was heated at 80° C. for 2 days. The crude reaction mixture was concentrated, then partitioned between EtOAc and water. The organic phase was treated with brine, dried (NaSO₄), filtered and concentrated. Purification by flash column on silica gel eluting with a gradient of hexanes: EtOAc provided 43 mg of cyclopropanecarboxylic acid {3-[2-(2-chloro-4-nitro-phenyl)-pyrrol-1-yl]-1H-pyrazolo [3,4-d]thiazol-5-yl}-amide as a yellow powder (35% yield): ¹H NMR (d₆-DMSO) δ 13.3 (broad s, 1H), 12.7 (broad s, 1H), 8.30 (d, 1H), 8.17 (dd, 1H), 7.59 (d, 1H), 7.40 (m, 1H), 6.55 (m, 1H), 6.45 (t, 1H), 1.91 (m, 1H), 0.91 (m, 2H), 0.87 (m, 2H); HPLC/MS m/z: 429 [MH]⁺.

Other compounds prepared by method X:

TABLE 11

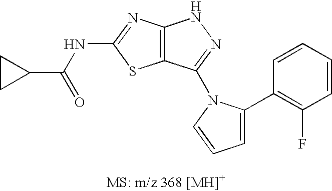

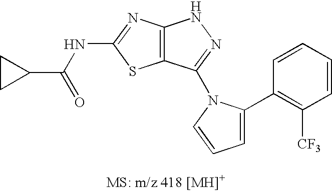

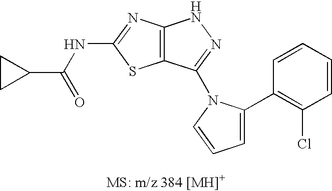

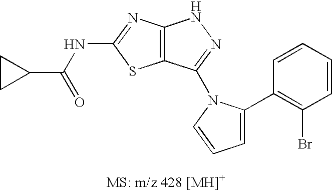

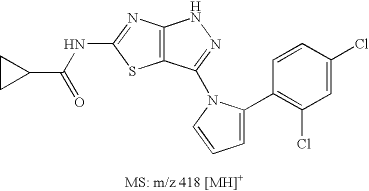

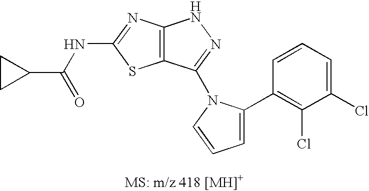

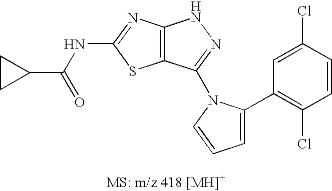

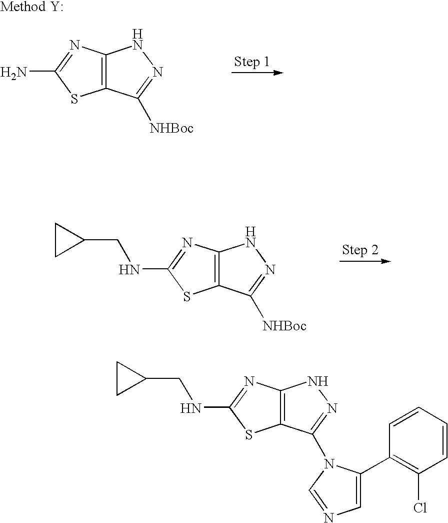

### Step 1: Synthesis of [5-(cyclopropylmethyl-amino)-1H-pyrazolo[3,4-d]thiazol-3-yl]-carbamic acid tert-butyl ester

To a solution of (5-amino-2H-pyrazol-3-yl)-carbamic acid tert-butyl ester (1 g, 3.92 mmol) in 1,2-dichloroethane, was added cyclopropyl aldehyde (0.55 g, 7.84 mmol). Acetic acid (0.235 g, 3.92 mmol) was then added and the mixture was allowed to stir for 30 min at room temperature. The mixture was then cooled to 0° C. and Na(OAc)₃BH (2.49 g, 11.76 mmol) was added portion-wise. The mixture was allowed to warm up to room temperature and stirred for 48 h. The solvent was removed in vacuo and the residue adsorbed onto silica gel. Purification on silica gel with 0-8% gradient of MeOH/CH₂Cl₂ as eluent provided 200 mg of [5-(cyclopropylmethyl-amino)-1H-pyrazolo[3,4-d]thiazol-3-yl]-carbamic acid tert-butyl ester (24% yield). HPLC/MS m/z: 210 [MH]⁺.

### Step 2: Synthesis of {3-[5-(2-chloro-phenyl)-imidazol-1-yl]-1H-pyrazolo[3,4-d]thiazol-5-yl}-cyclopropylmethyl-amine

To [5-(cyclopropylmethyl-amino)-1H-pyrazolo[3,4-d]thiazol-3-yl]-carbamic acid tert-butyl ester (114 mg, 0.368 mmol) was added PS-thiophenol (500 mg, 0.75 mmol, Argonaut resin) and trifluoroacetic acid (2.0 mL). The reaction mixture was stirred at room temperature for 2 h, and the resin was filtered and washed with MeOH. The filtrate was evaporated in vacuo, and dried to provide the TFA salt. To the TFA salt (0.368 mmol) was added absolute EtOH (1.0 mL) followed by 2-chlorobenzaldehyde (0.05 mL, 0.44 mmol). The reaction mixture was stirred at 80° C. for 16 h, and then dried in vacuo. The crude solid was dissolved in DMF (1.0 mL) and potassium carbonate (153 mg, 1.11 mmol) was added, followed by tosylmethyl isocyanide (94 mg, 0.48 mmol). The reaction mixture was stirred at 80° C. for 3 h, and then concentrated in vacuo. The crude solid was redissolved in 10% MeOH/CH₂Cl₂ and adsorbed onto silica gel. Purification on silica gel with 0-8% gradient of MeOH/CH₂Cl₂ as eluent provided 18 mg of {3-[5-(2-chloro-phenyl)-imidazol-1-yl]-1H-pyrazolo[3,4-d]thiazol-5-yl}-cyclopropylmethyl-amine (13% yield). ¹H NMR (d₆-DMSO) δ 12.8 (broad s, 1H), 8.03 (broad s, 1H), 7.98 (d, 1H), 7.38 (d, 1H), 7.28 (m, 2H), 7.23 (d, 1H), 6.98 (s, 1H), 2.90 (t, 2H), 0.85 (m, 1H), 0.27 (m, 2H), 0.02 (m, 2H); HPLC/MS m/z: 371 [MH]⁺.

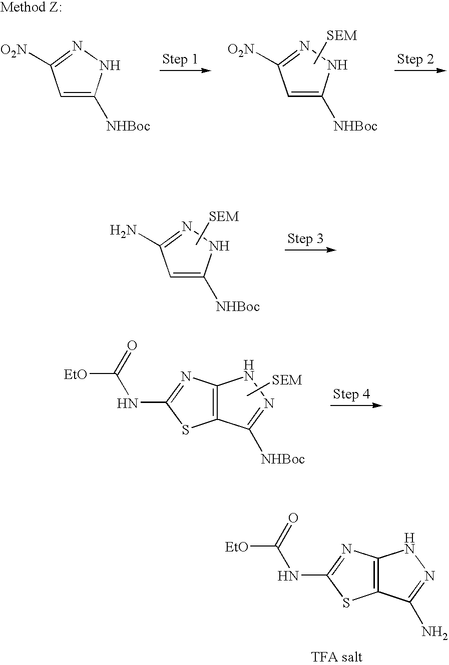

### Step 1: Synthesis of SEM-protected (5-nitro-2H-pyrazol-3-yl)-carbamic acid tert-butyl ester

A 500 mL round bottomed flask was charged with (5-nitro-1H-pyrazol-3-yl)-carbamic acid tert-butyl ester (10.0 g, 44 mmol) and dichloromethane (250 mL). A 4 N solution of KOH (55 mL, 220 mmol) was added under vigorous stirring. The solution was cooled in an ice bath at 0° C. and a solution of 2-(trimethylsilyl)ethoxymethyl chloride (11.64 mL, 66 mmol) in dichloromethane (100 mL) was added dropwise. After addition, the ice bath was removed and the reaction mixture was allowed to warm up to room temperature under stirring for 17 h. The reaction mixture was adjusted to pH 1-2 with 1 N aqueous solution of HCl and extracted with EtOAc (3×). The organic phase was washed with brine, dried (MgSO₄), filtered and concentrated in vacuo. The crude mixture of mono- and bis-protected (5-nitro-1H-pyrazol-3-yl)-carbamic acid tert-butyl ester (19.8 g) was used without purification for the next step. HPLC/MS: m/z 359 [MH]⁺ and m/z 489 [MH]⁺.

### Step 2: Synthesis of SEM-protected (5-amino-2H-pyrazol-3-yl)-carbamic acid tert-butyl ester

A 250 mL round bottomed flask was charged with the mixture of mono- and bis-protected (5-nitro-1H-pyrazol-3-yl)-carbamic acid tert-butyl ester (19.8 g, 44 mmol), 10%/wt Pd/C (4.4 g, 4.4 mmol) and methanol (180 mL). The flask was purged with hydrogen gas. After 22 h under hydrogen atmosphere, the mixture reaction was filtered through celite and concentrated in vacuo. The mixture of mono- and bis-protected (5-amino-2H-pyrazol-3-yl)-carbamic acid tert-butyl ester (18.2 g) was used without purification for the next step. HPLC/MS: m/z 329 [MH]⁺ and m/z 459 [MH]⁺.

### Step 3: Synthesis of SEM-protected (5-ethoxycarbonylamino-1H-pyrazolo[3,4-d]thiazol-3-yl)-carbamic acid tert-butyl ester

To a solution of mixture of mono- and bis-protected (5-amino-2H-pyrazol-3-yl)-carbamic acid tert-butyl ester (18.2 g, 40 mmol) in THF (400 mL) was added ethoxycarbonyl isothiocyanate (4.52 mL, 40 mmol) dropwise. The reaction was stirred at room temperature for 2 h until completion. A solution of NBS (7.83 mmol, 44 mmol) in THF (100 mL) was added dropwise at room temperature. After 15 min, the reaction mixture was cooled in an ice bath at 0° C. and quenched with a saturated aqueous solution of NaHCO₃ and extracted 3 times with EtOAc. The organic phase was washed with brine, dried (Na₂SO₄), filtered and concentrated in vacuo. The resulting solid was recrystallized in EtOAc/Hexane to afford 3.01 g of the mono-protected title compound as a tan solid. The filtrate was purified on normal phase silica using EtOAc/Hexane to afford another 3.0 g. HPLC/MS: m/z 458 [MH]⁺.

### Step 4: Synthesis of (3-amino-1H-pyrazolo[3,4-d]thiazol-5-yl)-carbamic acid ethyl ester, trifluoroacetic acid salt

A 250 mL round bottomed flask was charged with SEM-protected (5-ethoxycarbonylamino-1H-pyrazolo[3,4-d]thiazol-3-yl)-carbamic acid tert-butyl ester (4.33 g, 9.47 mmol), PS-thiophenol resin (19.0 g, 28.4 mmol, Argonaut resin), and dichloromethane (80 ml). The suspension was treated with trifluoroacetic acid (15 ml) and the reaction mixture was stirred at room temperature for 21 h. The resin was then filtered and washed with methanol. The filtrate was concentrated in vacuo and dried under high vacuum to afford 1.43 g of tan powder of (3-amino-1H-pyrazolo[3,4-d]thiazol-5-yl)-carbamic acid ethyl ester, trifluoroacetic acid salt, as a white solid (44% yield). ¹H NMR (d₆-DMSO) δ 12.4 (br s, 1H), 4.25 (q, 2H), 1.25 (t, 3H); HPLC/MS m/z: 228 [MH]⁺.

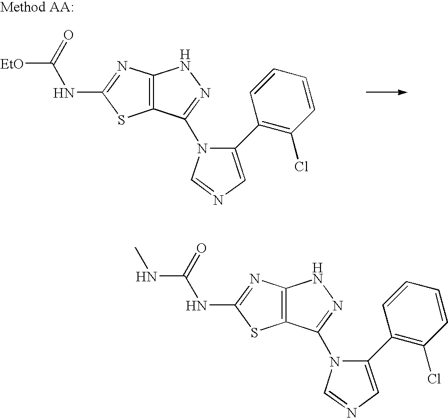

### Synthesis of 1-{3-[5-(2-chloro-phenyl)-imidazol-1-yl]-1H-pyrazolo [3,4-d]thiazol-5-yl}-3-methyl-urea

A microwave vessel was charged with {3-[5-(2-chloro-phenyl)-imidazol-1-yl]-1H-pyrazolo[3,4-d]thiazol-5-yl}-carbamic acid ethyl ester (15 mg, 0.0385 mmol) and ethanol (0.4 mL). Methylamine (18 uL, 0.523 mmol) was added, the vessel was sealed and heated in a Personal Chemistry microwave reactor at 160° C. for 65 min. The crude reaction mixture was diluted to 1 mL with DMSO, filtered through a 0.45 um syringe filter and purified by mass-triggered reverse phase preparative HPLC in a mobile phase of H₂O and acetonitrile (with ammonium bicarbonate as the modifier). Clean fractions were combined and lyophilized, affording 4.0 mg of 1-{3-[5-(2-chloro-phenyl)-imidazol-1-yl]-1H-pyrazolo[3,4-d]thiazol-5-yl}-3-methyl-urea as an off-white solid (28% yield). ¹H NMR (d₆-DMSO) δ 8.14 (d, 1H), 7.48 (d, 1H), 7.36-7.44 (m, 3H), 7.16 (d, 1H), 6.44 (q, 1H), 2.65 (d, 3H); HPLC/MS m/z: 374 [MH]⁺.

Other compounds prepared by method AA:

TABLE 12

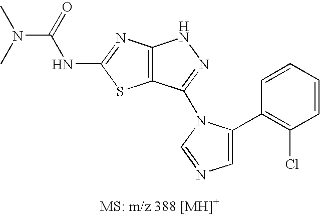

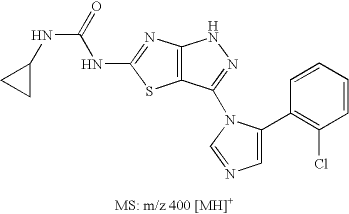

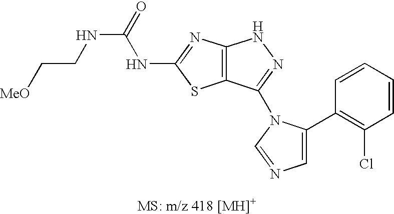

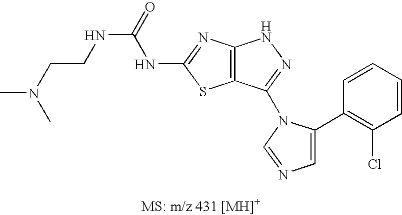

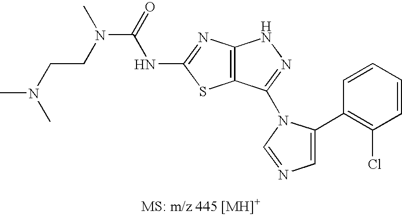

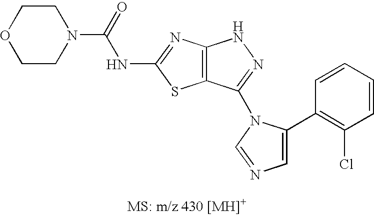

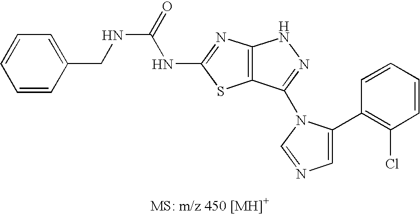

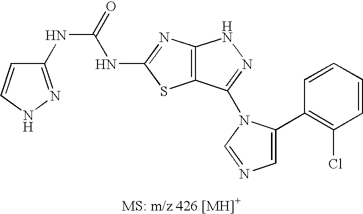

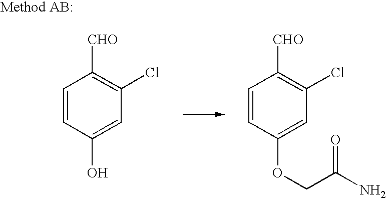

### Synthesis of 2-(3-chloro-4-formyl-phenoxy)-acetamide

A vial was charged with 2-chloro-4-hydroxybenzaldehyde (60 mg, 0.383 mmol), 2-bromoacetamide (58 mg, 0.421 mmol), cesium carbonate (374 mg, 1.149 mmol), and a few crystals of potassium iodide [potassium carbonate and sodium iodide are good substitutes for a base and catalyst, respectively]. DMF (1 mL) was added and the reaction mixture was stirred at room temperature overnight [heating is optional]. The mixture was concentrated in vacuo, diluted in MeOH and directly adsorbed on silica gel. Purification on silica gel with 0-10% gradient of MeOH/CH₂Cl₂ as eluent provided 32 mg of 2-(3-chloro-4-formyl-phenoxy)-acetamide as a white solid (39% yield). ¹H NMR (d₆-DMSO) δ 10.2 (s, 1H), 7.84 (d, 1H), 7.63 (broad s, 1H), 7.45 (broad s, 1H), 7.17 (d, 1H), 7.10 (dd, 1H), 4.61 (s, 2H).

Other aldehydes prepared by method AB:

TABLE 13

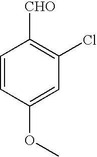

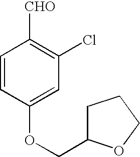

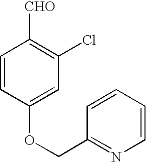

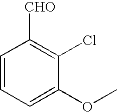

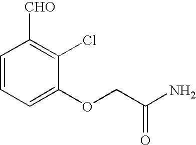

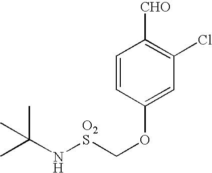

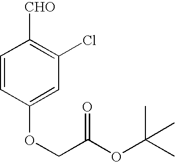

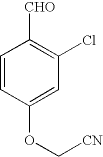

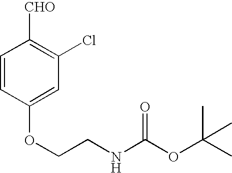

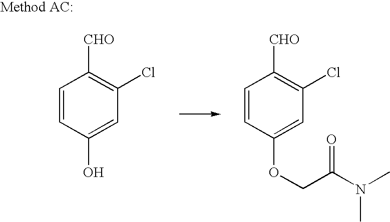

### Synthesis of 2-(3-chloro-4-formyl-phenoxy)-N,N-dimethyl-acetamide

A microwave vial was charged with 2-chloro-4-hydroxybenzaldehyde (50 mg, 0.319 mmol), N,N-dimethyl-2-chloroacetamide (36 uL, 0.35 mmol), cesium carbonate (312 mg, 0.957 mmol), and a few crystals of sodium iodide [potassium carbonate and potassium iodide are good substitutes for a base and catalyst, respectively]. DMF (1.5 mL) was added and the reaction mixture was run on a Personal Chemistry microwave reactor at 150° C. for 900 seconds. Cesium carbonate was filtered and the filtrate was adsorbed directly on silica gel. Purification on silica gel with 20-100% gradient of EtOAc/Hexane as eluent provided 53 mg of 2-(3-chloro-4-formyl-phenoxy)-N,N-dimethyl-acetamide as a clear oil (69% yield). ¹H NMR (d₆-DMSO) δ 10.2 (s, 1H), 7.80 (d, 1H), 7.16 (d, 1H), 7.03 (dd, 1H), 5.03 (s, 2H), 2.97 (s, 3H), 2.84 (s, 3H).

Other aldehydes prepared by method AC:

TABLE 14

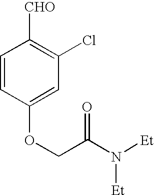

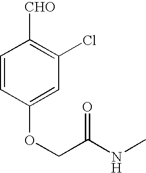

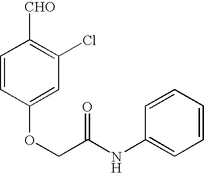

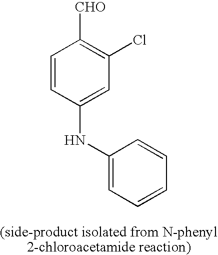

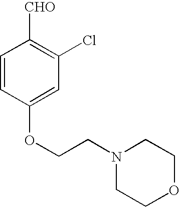

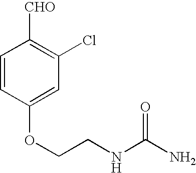

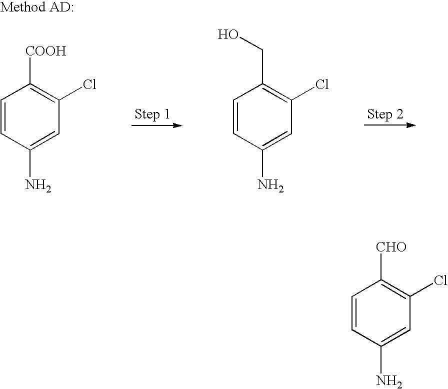

### Step 1: Synthesis of (4-amino-2-chloro-phenyl)-methanol

To a 1 M solution of lithium aluminum hydride in THF (58 mL) under nitrogen atmosphere was added a solution of 4-amino-2-chloro-benzoic acid (5 g, 29.14 mmol) in THF (40 mL) dropwise at 0° C. Ice bath was removed and the reaction mixture was stirred at room temperature overnight, then at reflux for 2 h. The reaction was quenched at 0° C. by adding water (2.35 mL) then 5% aqueous sodium hydroxide (7.2 mL) dropwise. The temperature was allowed to rise to room temperature over the course of 1 h. The resulting precipitate was filtered, washed with EtOAc, and the filtrate was adsorbed directly on silica gel. Purification on silica gel with 0-80% gradient of EtOAc/Hexane as eluent provided 2.57 g of (4-amino-2-chloro-phenyl)-methanol as an off-white solid (56% yield). ¹H NMR (d₆-DMSO) δ 7.10 (d, 1H), 6.56 (d, 1H), 6.47 (dd, 1H), 5.24 (broad s, 2H), 4.92 (t, 1H), 4.36 (d, 2H).

### Step 2: Synthesis of 4-amino-2-chloro-benzaldehyde

To a solution of (4-amino-2-chloro-phenyl)-methanol (2.5 g, 15.86 mmol) in dichloromethane (150 mL) was added MnO₂ (13.8 g, 158.6 mmol) in one portion. The reaction mixture was stirred at room temperature for 23 h, then it was filtered over celite. The filtrate was adsorbed directly on silica gel. Purification on silica gel with 0-60% gradient of EtOAc/Hexane as eluent provided 726 mg of 4-amino-2-chloro-benzaldehyde as an orange-yellow solid (29% yield). ¹H NMR (d₆-DMSO) δ 9.95 (s, 1H), 7.56 (d, 1H), 6.62 (broad s, 1H), 6.60 (d, 1H), 6.56 (dd, 1H).

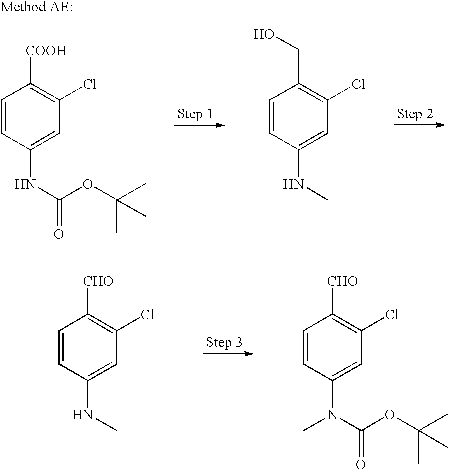

### Step 1: Synthesis of (2-chloro-4-methylamino-phenyl)-methanol

To a 1 M solution of lithium aluminum hydride in THF (7.48 mL) under nitrogen atmosphere was added a solution of 4-tert-Butoxycarbonylamino-2-chloro-benzoic acid (1 g, 3.68 mmol) in THF (5 mL) dropwise at 0° C. Ice bath was removed and the reaction mixture was stirred at room temperature for 2 h, then at 5° C. for 2 h. The reaction was quenched at 0° C. by adding water (0.3 mL) then 5% aqueous sodium hydroxide (0.92 mL) dropwise. EtOAc was added and the precipitate was filtered, and washed with EtOAc. The filtrate was further washed with a saturated aqueous solution of sodium bicarbonate (2×) and brine. The organic layer was dried over Na₂SO₄, filtered and adsorbed directly on silica gel. Purification on silica gel with 0-100% gradient of EtOAc/Hexane as eluent provided 390 mg of (2-chloro-4-methylamino-phenyl)-methanol as a white waxy solid (62% yield). ¹H NMR (d₆-DMSO) δ 7.16 (d, 1H), 6.48 (d, 1H), 6.46 (dd, 1H), 5.82 (q, 1H), 4.93 (t, 1H), 4.38 (d, 2H), 2.63 (d, 3H).

### Step 2: Synthesis of 2-chloro-4-methylamino-benzaldehyde

To a solution of(2-chloro-4-methylamino-phenyl)-methanol (380 mg, 2.21 mmol) in chloroform (20 mL) was added MnO₂ (1.9 g, 22.1 mmol) in one portion. The reaction mixture was stirred at room temperature until completion, then it was filtered over celite. The filtrate was adsorbed directly on silica gel. Purification on silica gel with 0-70% gradient of EtOAc/Hexane as eluent provided 296 mg of 2-chloro-4-methylamino-benzaldehyde as a yellow solid (79% yield). ¹H NMR (d₆-DMSO) δ 9.81 (s, 1H), 7.60 (d, 1H), 7.19 (q, 1H), 6.58 (m, 2H), 2.76 (d, 3H).

### Step 3: Synthesis of (3-chloro-4-formyl-phenyl)-methyl-carbamic acid tert-butyl ester

To a solution of 2-chloro-4-methylamino-benzaldehyde (290 mg, 1.71 mmol) in DMF (10 mL) was added 4-dimethylaminopyridine (209 mg, 1.71 mmol) followed by di-tert-butyloxycarbonyl anhydride (410 mg, 1.88 mmol). The reaction mixture was stirred at 80° C. for 2.5 h, then it was concentrated in vacuo and diluted with EtOAc. The organics were washed with 1 N aqueous HCl (2×) and brine. The organic layer was dried over Na₂SO₄, filtered, concentrated and dried in vacuo to provide 444 mg of (3-chloro-4-formyl-phenyl)-methyl-carbamic acid tert-butyl ester as a yellow oil (96% yield). ¹H NMR (d₆-DMSO) δ 10.2 (s, 1H), 7.82 (d, 1H), 7.61 (d, 1H), 7.48 (dd, 1H), 3.26 (s, 3H), 1.44 (s, 9H).

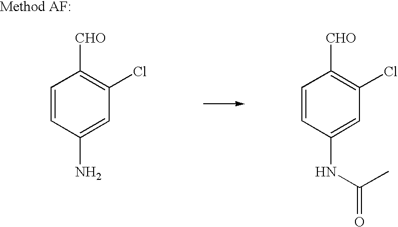

### Synthesis of N-(3-chloro-4-formyl-phenyl)-acetamide

To a solution of 4-amino-2-chloro-benzaldehyde (30 mg, 0.193 mmol) in pyridine (0.5 mL) was added acetyl chloride (30 uL, 0.414 mmol) dropwise. The reaction mixture was stirred at 60° C. for 8 h, then concentrated in vacuo. The crude was partitioned between EtOAc and a saturated aqueous solution of copper (II) sulfate. The organic layer was washed with water and adsorbed directly on silica gel. Purification on silica gel with 0-70% gradient of EtOAc/Hexane as eluent provided 26 mg of N-(3-chloro-4-formyl-phenyl)-acetamide as a beige solid (68% yield). ¹H NMR (d₆-DMSO) δ 10.5 (s, 1H), 10.2 (s, 1H), 7.96 (s, 1H), 7.83 (d, 1H), 7.57 (d, 1H), 2.10 (s, 3H).

Other aldehyde prepared by method AF:

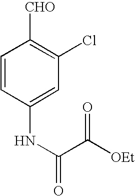

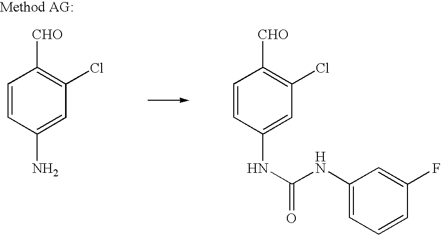

### Synthesis of 1-(3-chloro-4-formyl-phenyl)-3-(3-fluoro-phenyl)-urea

To a suspension of 4-amino-2-chloro-benzaldehyde (30 mg, 0.193 mmol) in toluene (0.5 mL) was added 3-fluorophenyl isocyanate (24 uL, 0.212 mL). The reaction mixture was stirred at 60° C. for 3 days, then diluted in MeOH and adsorbed on silica gel. Purification on silica gel with 0-10% gradient of MeOH/CH₂Cl₂ as eluent provided 38 mg of 1-(3-chloro-4-formyl-phenyl)-3-(3-fluoro-phenyl)-urea as a dark yellow solid (67% yield). ¹H NMR (d₆-DMSO) δ 10.2 (s, 1H), 9.46 (s, 1H), 9.18 (s, 1H), 7.85 (d, 1H), 7.81 (d, 1H), 7.47 (dt, 1H), 7.43 (dd, 1H), 7.33 (dd, 1H), 7.16 (d, 1H), 6.83 (td, 1H).

Other aldehyde prepared by method AG:

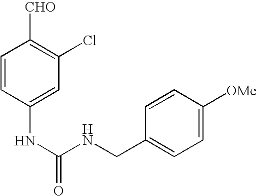

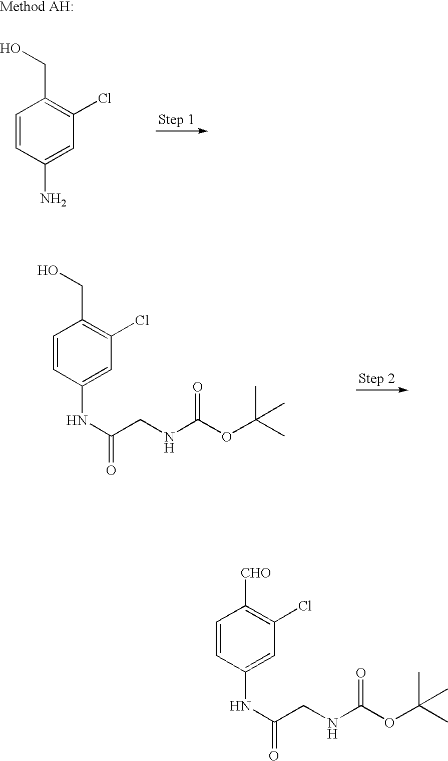

### Step 1: Synthesis of [(3-chloro-4-hydroxymethyl-phenylcarbamoyl)-methyl]-carbamic acid tert-butyl ester

A vial was charged with (4-amino-2-chloro-phenyl)-methanol (50 mg, 0.317 mmol) and Boc-glycine (56 mg, 0.317 mmol). Dichloromethane (1 mL) was added, followed by diisopropylethylamine (61 uL, 0.349 mmol) and N-(3-dimethylaminopropyl)-N′-ethylcarbodiimide hydrochloride (67 mg, 0.349 mmol). The reaction mixture was stirred at room temperature overnight, then 1 N aqueous solution of sodium hydroxide (1 mL) was added and the mixture was stirred for another hour. The organic layer was separated, washed with 1 N aqueous solution of sodium hydroxide, 1 N aqueous solution of HCl, and brine. The organic layer was dried over Na₂SO₄, filtered, concentrated and dried in vacuo to provide 43 mg of [(3-chloro-4-hydroxymethyl-phenylcarbamoyl)-methyl]-carbamic acid tert-butyl ester as a slightly pink foam (44% yield). ¹H NMR (d₆-DMSO) δ 10.1 (s, 1H), 7.77 (s, 1H), 7.44 (s, 2H), 7.06 (t, 1H), 5.29 (t, 1H), 4.48 (d, 2H), 3.70 (d, 2H), 1.38 (s, 9H).

### Step 2: Synthesis of [(3-chloro-4-formyl-phenylcarbamoyl)-methyl]-carbamic acid tert-butyl ester

To a solution of [(3-chloro-4-hydroxymethyl-phenylcarbamoyl)-methyl]-carbamic acid tert-butyl ester (40 mg, 0.127 mmol) in chloroform (1 mL) was added MnO₂ (110 mg, 1.27 mmol) in one portion. The reaction mixture was stirred at room temperature until completion, then it was filtered over celite. The filtrate was adsorbed directly on silica gel. Purification on silica gel with 0-80% gradient of EtOAc/Hexane as eluent provided 22 mg of [(3-chloro-4-formyl-phenylcarbamoyl)-methyl]-carbamic acid tert-butyl ester as a foam (55% yield). ¹H NMR (d₆-DMSO) δ 10.5 (s, 1H), 10.2 (s, 1H), 7.95 (d, 1H), 7.84 (d, 1H), 7.59 (d, 1H), 7.14 (t, 1H), 3.75 (d, 2H), 1.38 (s, 9H).

Other aldehyde prepared by method AH:

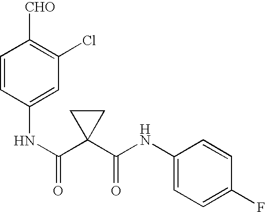

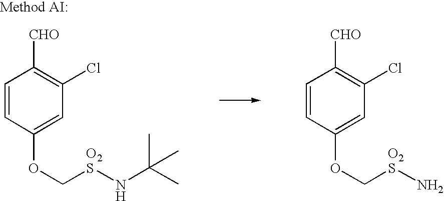

### Synthesis of (3-chloro-4-formyl-phenoxy)-methanesulfonamide

To a solution of N-tert-butyl-C-(3-chloro-4-formyl-phenoxy)-methanesulfonamide (156 mg, 0.511 mmol) in 1,4-dioxane (2.7 mL) was added 6 N aqueous HCl (2.7 mL) dropwise. The reaction mixture was stirred at 90° C. for 1.5 h, then it was diluted with water and extracted EtOAc (3×). The combined extracts were adsorbed on silica gel. Purification on silica gel with 0-70% gradient of EtOAc/Hexane as eluent provided 73 mg of (3-chloro-4-formyl-phenoxy)-methanesulfonamide as a white solid (57% yield). ¹H NMR (d₆-DMSO) δ 10.2 (s, 1H), 7.85 (d, 1H), 7.41 (d, 1H), 7.28 (broad s, 2H), 7.25 (dd, 1H), 5.27 (s, 2H).

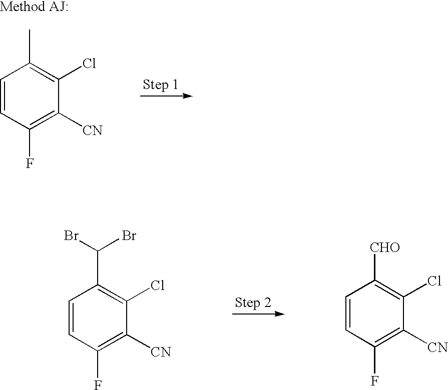

### Step 1: Synthesis of 2-chloro-3-dibromomethyl-6-fluoro-benzonitrile

To a solution of 2-chloro-6-fluoro-3-methyl-benzonitrile (2 g, 11.79 mmol) in carbon tetrachloride (60 mL) under nitrogen atmosphere was added N-bromosuccinimide (6.3 g, 35.4 mmol) and benzoyl peroxide (286 mg, 1.18 mmol). The reaction mixture was stirred at reflux for 22 h then concentrated in vacuo. The residue was partitioned between EtOAc and a saturated aqueous solution of sodium bicarbonate. The organic layer was further washed with a saturated aqueous solution of sodium bicarbonate (2×) and brine, then it was dried over Na₂SO₄, filtered, and adsorbed on silica gel. Purification on silica gel with 0-25% gradient of EtOAc/Hexane as eluent provided 3.05 g of 2-chloro-3-dibromomethyl-6-fluoro-benzonitrile as a clear oil (79% yield). ¹H NMR (d₆-DMSO) δ 8.32 (dd, 1H), 7.69 (t, 1H), 7.52 (s, 1H).

### Step 2: Synthesis of 2-chloro-6-fluoro-3-formyl-benzonitrile

2-Chloro-3-dibromomethyl-6-fluoro-benzonitrile (1 g, 3.06 mmol) was treated with concentrated sulfuric acid (10 mL). The reaction mixture was stirred at 45° C. for 21 h, then poured onto ice. A 4 N aqueous solution of sodium hydroxide was added until pH 4. The aqueous solution was extracted with EtOAc (3×), and the combined organic layers were adsorbed on silica gel. Purification on silica gel with 0-80% gradient of EtOAc/Hexane as eluent provided 360 mg of 2-chloro-6-fluoro-3-formyl-benzonitrile as a white solid (64% yield). ¹H NMR (d₆-DMSO) δ 10.3 (s, 1H), 8.24 (broad s, 1H), 8.01 (broad s, 1H), 7.95 (dd, 1H), 7.50 (t, 1H).

### Example 2

### Bioassays

Kinase assays known to those of skill in the art may be used to assay the inhibitory activities of the compounds and compositions of the present invention. Kinase assays include, but are not limited to, the following examples.

Homogeneous luminescence-based inhibitor screening assays were developed for c-Abl, MET, AurA, and PDK1 kinases (among others). Each of these assays made use of an ATP depletion assay (Kinase-Glo™, Promega Corporation, Madison, Wis.) to quantitate kinase activity. The Kinase-Glo™ format uses a thermostable luciferase to generate luminescent signal from ATP remaining in solution following the kinase reaction. The luminescent signal is inversely correlated with the amount of kinase activity.

Screening data was evaluated using the equation: Z′=1−[3*(σ₊+σ₋)/|μ₊−μ₋|](Zhang, et al., 1999 J Biomol Screening 4(2) 67-73), where μ denotes the mean and σ the standard deviation. The subscript designates positive or negative controls. The Z′ score for a robust screening assay should be ≧0.50. The typical threshold=μ₊−3*σ₊. Any value that falls below the threshold was designated a “hit”.

Dose response was analyzed using the equation: y=min+{(max−min)/(1 +10^{[compound]-logIC}50)}, where y is the observed initial slope, max=the slope in the absence of inhibitor, min=the slope at infinite inhibitor, and the IC₅₀ is the [compound] that corresponds to ½ the total observed amplitude (Amplitude=max−min).

Preparation of Co-expression Plasmid

A lambda phosphatase co-expression plasmid was constructed as follows.

An open-reading frame for Aurora kinase was amplified from a Homo sapiens (human) HepG2 cDNA library (ATCC HB-8065) by the polymerase chain reaction (PCR) using the following primers:

Forward primer:
TCAAAAAAGAGGCAGTGGGCTTTG
Reverse primer:
CTGAATTTGCTGTGATCCAGG.

The PCR product (795 base pairs expected) was gel purified as follows. The PCR product was purified by electrophoresis on a 1% agarose gel in TAE buffer and the appropriate size band was excised from the gel and eluted using a standard gel extraction kit. The eluted DNA was ligated for 5 minutes at room temperature with topoisomerase into pSB2-TOPO. The vector pSB2-TOPO is a topoisomerase-activated, modified version of pET26b (Novagen, Madison, WI) wherein the following sequence has been inserted into the NdeI site: CATAATGGGCCATCATCATCATCATCACGGT GGTCATATGTCCCTT and the following sequence inserted into the BamHI site:

AAGGGGGATCCTAAACTGCAGAGATCC.

The sequence of the resulting plasmid, from the Shine-Dalgarno sequence through the “original” NdeI site, the stop site and the “original” BamHI site is as follows: AAGGAGGAGATATACATAATGGGCCATCATCATCATCATCACGGTGGTCATATGT CCCTT [ORF] AAGGGGGATCCTAAACTGCAGAGATCC. The Aurora kinase expressed using this vector has 14 amino acids added to the N-terminus (MetGlyHisHisHisHisHisHisGlyGlyHisMetSerLeu) and four amino acids added to the C-terminus (GluGlyGlySer).

The phosphatase co-expression plasmid was then created by inserting the phosphatase gene from lambda bacteriophage into the above plasmid (Matsui T, et al., Biochem. Biophys. Res. Commun., 2001, 284:798-807). The phosphatase gene was amplified using PCR from template lambda bacteriophage DNA (HinDIII digest, New England Biolabs) using the following oligonucleotide primers:

Forward primer (PPfor):
GCAGAGATCCGAATTCGAGCTCCGTCGACGGATGGAGTGAAAGAGATGCG
C
Reverse primer (PPrev):
GGTGGTGGTGCTCGAGTGCGGCCGCAAGCTTTCATCATGCGCCTTCTCCC
TGTAC.

The PCR product (744 base pairs expected) was gel purified. The purified DNA and non-co-expression plasmid DNA were then digested with SacI and XhoI restriction enzymes. Both the digested plasmid and PCR product were then gel purified and ligated together for 8 h at 16° C. with T4 DNA ligase and transformed into Top 10 cells using standard procedures. The presence of the phosphatase gene in the co-expression plasmid was confirmed by sequencing. For standard molecular biology protocols followed here, see also, for example, the techniques described in Sambrook et al., Molecular Cloning: A Laboratory Manual, Cold Spring Harbor Laboratory, NY, 2001, and Ausubel et al., Current Protocols in Molecular Biology, Greene Publishing Associates and Wiley Interscience, NY, 1989.

This co-expression plasmid contains both the Aurora kinase and lambda phosphatase genes under control of the lac promoter, each with its own ribosome binding site. By cloning the phosphatase into the middle of the multiple cloning site, downstream of the target gene, convenient restriction sites are available for subcloning the phosphatase into other plasmids. These sites include SacI, SalI and EcoRI between the kinase and phosphatase and HinDIII, NotI and XhoI downstream of the phosphatase.

Protein Kinase Expression

An open-reading frame for c-Abl was amplified from a Mus musculus (mouse) cDNA library prepared from freshly harvested mouse liver using a commercially available kit (Invitrogen) by PCR using the following primers:

Forward primer:
GACAAGTGGGAAATGGAGC
Reverse primer:
CGCCTCGTTTCCCCAGCTC.

The PCR product (846 base pairs expected) was purified from the PCR reaction mixture using a PCR cleanup kit (Qiagen). The purified DNA was ligated for 5 minutes at room temperature with topoisomerase into pSGX3-TOPO. The vector pSGX3-TOPO is a topoisomerase-activated, modified version of pET26b (Novagen, Madison, Wisconsin) wherein the following sequence has been inserted into the NdeI site: CATATGTCCCTT and the following sequence inserted into the BamHI site:

AAGGGCATCATCACCATCACCACTGATCC.

The sequence of the resulting plasmid, from the Shine-Dalgarno sequence through the stop site and the BamHI, site is as follows: AAGGAGGA GATATACATATGTC CCTT[ORF]AAGGGCATCAT CACCATCACCACTGATCC. The c-Abl expressed using this vector had three amino acids added to its N-terminus (Met Ser Leu) and 8 amino acids added to its C-terminus (GluGlyHisHisHisHisHisHis).

A c-Abl/phosphatase co expression plasmid was then created by subcloning the phosphatase from the Aurora co-expression plasmid into the above plasmid. Both the Aurora co-expression plasmid and the Abl non-co-expression plasmid were digested 3 hrs with restriction enzymes EcoRI and NotI. The DNA fragments were gel purified and the phosphatase gene from the Aurora plasmid was ligated with the digested c-Abl plasmid for 8 h at 16° C. and transformed into Top 10 cells. The presence of the phosphatase gene in the resulting construct was confirmed by restriction digestion analysis.

This plasmid codes for c-Abl and lambda phosphatase co expression. It has the additional advantage of two unique restriction sites, XbaI and NdeI, upstream of the target gene that can be used for subcloning of other target proteins into this phosphatase co-expressing plasmid.

The plasmid for Abl T315I was prepared by modifying the Abl plasmid using the Quick Change mutagenesis kit (Stratagene) with the manufacturer's suggested procedure and the following oligonucleotides:

Mm05582dS4
5′-CCACCATTCTACATAATCATTGAGTTCATGACCTATGGG-3′
Mm05582dA4
5′-CCCATAGGTCATGAACTCAATGATTATGTAGAATGGTGG-3′.

Protein from the phosphatase co-expression plasmids was purified as follows. The non-co-expression plasmid was transformed into chemically competent BL21(DE3)Codon+RIL (Stratagene) cells and the co-expression plasmid was transformed into BL21(DE3) pSA0145 (a strain that expresses the lytic genes of lambda phage and lyses upon freezing and thawing (Crabtree S, Cronan JE Jr. J Bacteriol 1984 Apr;158(1):354-6)) and plated onto petri dishes containing LB agar with kanamycin. Isolated single colonies were grown to mid-log phase and stored at −80° C. in LB containing 15% glycerol. This glycerol stock was streaked on LB agar plates with kanamycin and a single colony was used to inoculate 10 ml cultures of LB with kanamycin and chloramphenicol, which was incubated at 30° C. overnight with shaking. This culture was used to inoculate a 2 L flask containing 500 ml of LB with kanamycin and chloramphenicol, which was grown to mid-log phase at 37° C. and induced by the addition of IPTG to 0.5 mM final concentration. After induction flasks were incubated at 21° C. for 18 h with shaking.

The c-Abl T315I KD (kinase domain) was purified as follows. Cells were collected by centrifugation, lysed in diluted cracking buffer (50 mM Tris HCl, pH 7.5, 500 mM KCl, 0.1% Tween 20, 20 mM Imidazole, with sonication, and centrifuged to remove cell debris. The soluble fraction was purified over an IMAC column charged with nickel (Pharmacia, Uppsala, Sweden), and eluted under native conditions with a gradient of 20 mM to 500 mM imidazole in 50 mM Tris, pH7.8, 500 mM NaCl, 10 mM methionine, 10% glycerol. The protein was then further purified by gel filtration using a Superdex 75 preparative grade column equilibrated in GF5 buffer (10 mM HEPES, pH7.5, 10 mM methionine, 500 mM NaCl, 5 mM DTT, and 10% glycerol). Fractions containing the purified c-Abl T315I KD kinase domain were pooled. The protein obtained was 98% pure as judged by electrophoresis on SDS polyacrylamide gels. Mass spectroscopic analysis of the purified protein showed that it was predominantly singly phosphorylated. The protein was then dephosphorylated with Shrimp Alkaline Phosphatase (MBI Fermentas, Burlington, Canada) under the following conditions: 100U Shrimp Alkaline Phosphatase/mg of c-Abl T315I KD, 100 mM MgCl₂, and 250 mM additional NaCl. The reaction was run overnight at 23° C. The protein was determined to be unphosphorylated by Mass spectroscopic analysis. Any precipitate was spun out and the soluble fraction was separated from reactants by gel filtration using a Superdex 75 preparative grade column equilibrated in GF4 buffer (10 mM HEPES, pH7.5, 10 mM methionine, 150 mM NaCl, 5 mM DTT, and 10% glycerol).

Purification of Met:

The cell pellets produced from half of a 12 L Sf9 insect cell culture expressing the kinase domain of human Met were resuspended in a buffer containing 50 mM Tris-HCl pH 7.7 and 250 mM NaCl, in a volume of approximately 40 ml per 1 L of original culture. One tablet of Roche Complete, EDTA-free protease inhibitor cocktail (Cat# 1873580) was added per 1 L of original culture. The suspension was stirred for 1 hour at 4° C. Debris was removed by centrifugation for 30 minutes at 39,800×g at 4° C. The supernatant was decanted into a 500 ml beaker and 10 ml of 50% slurry of Qiagen Ni-NTA Agarose (Cat# 30250) that had been pre-equilibrated in 50 mM Tris-HCl pH 7.8, 50 mM NaCl, 10% Glycerol, 10 mM Imidazole, and 10 mM Methionine, were added and stirred for 30 minutes at 4° C. The sample was then poured into a drip column at 4° C. and washed with 10 column volumes of 50 mM Tris-HCl pH 7.8, 500 mM NaCl, 10% Glycerol, 10 mM Imidazole, and 10 mM Methionine. The protein was eluted using a step gradient with two column volumes each of the same buffer containing 50 mM, 200 mM, and 500 mM Imidazole, sequentially. The 6× Histidine tag was cleaved overnight using 40 units of TEV protease (Invitrogen Cat# 10127017) per 1 mg of protein while dialyzing in 50 mM Tris-HCl pH 7.8, 500 mM NaCl, 10% Glycerol, 10 mM Imidazole, and 10 mM Methionine at 4° C. The 6× Histidine tag was removed by passing the sample over a Pharmacia 5 ml IMAC column (Cat# 17-0409-01) charged with Nickel and equilibrated in 50 mM Tris-HCl pH 7.8, 500 mM NaCl, 10% Glycerol, 10 mM Imidazole, and 10 mM Methionine. The cleaved protein bound to the Nickel column at a low affinity and was eluted with a step gradient. The step gradient was run with 15% and then 80% of the B-side (A-side=50 mM Tris-HCl pH 7.8, 500 mM NaCl, 10% Glycerol, 10 mM Imidazole, and 10 mM Methionine; B-side=50 mM Tris-HCl pH 7.8, 500 mM NaCl, 10% Glycerol, 500 mM Imidazole, and 10 mM Methionine) for 4 column volumes each. The Met protein eluted in the first step (15%), whereas the non-cleaved Met and the cleaved Histidine tag eluted in the 80% fractions. The 15% fractions were pooled after SDS-PAGE gel analysis confirmed the presence of cleaved Met; further purification was done by gel filtration chromatography on an Amersham Biosciences HiLoad 16/60 Superdex 200 prep grade (Cat# 17-1069-01) equilibrated in 50 mM Tris-HCl pH 8.5, 150 mM NaCl, ˜10% Glycerol and 5 mM DTT. The cleanest fractions were combined and concentrated to ˜10.4 mg/ml by centrifugation in an Amicon Ultra-15 10,000 Da MWCO centrifugal filter unit (Cat# UFC901024).

Purification of AurA:

The Sf9 insect cell pellets (˜18 g) produced from 6 L of cultured cells expressing human Aurora-2 were resuspended in 50 mM Na Phosphate pH 8.0, 500 mM NaCl, 10% glycerol, 0.2% n-octyl-β-D-glucopyranoside (BOG) and 3 mM β-Mercaptoethanol (BME). One tablet of Roche Complete, EDTA-free protease inhibitor cocktail (Cat# 1873580) and 85 units Benzonase (Novagen Cat#70746-3)) were added per 1 L of original culture. Pellets were resuspended in approximately 50 ml per 1 L of original culture and were then sonicated on ice with two 30-45 sec bursts (100% duty cycle). Debris was removed by centrifugation and the supernatant was passed through a 0.8 μm syringe filter before being loaded onto a 5 ml Ni²⁺ HiTrap column (Pharmacia). The column was washed with 6 column volumes of 50 mM Na Phosphate pH 8.0, 500 mM NaCl, 10% glycerol, 3 mM BME. The protein was eluted using a linear gradient of the same buffer containing 500 mM Imidazole. The eluant (24 ml) was cleaved overnight at 4° C. in a buffer containing 50 mM Na Phosphate pH 8.0, 500 mM NaCl, 10% glycerol, 3 mM BME and 10,000 units of TEV (Invitrogen Cat#10127-017). The protein was passed over a second nickel affinity column as described above; the flow-through was collected. The cleaved protein fractions were combined and concentrated using spin concentrators. Further purification was done by gel filtration chromatography on a S75 sizing column in 50 mM Na Phosphate (pH 8.0), 250mM NaCl, 1 mM EDTA, 0.1 mM AMP-PNP or ATP buffer, and 5 mM DTT. The cleanest fractions were combined and concentrated to approximately 8-11 mg/ml, and were either flash frozen in liquid nitrogen in 120 μl aliquots and stored at −80° C., or stored at 4° C.

Purification of PDK1:

Cell pellets produced from 6 L of Sf9 insect cells expressing human PDK1 were resuspended in a buffer containing 50 mM Tris-HCl pH 7.7 and 250 mM NaCl in a volume of approximately 40 mL per 1 L of original culture. One tablet of Roche Complete, EDTA-free protease inhibitor cocktail (Cat# 1873580) and 85 units Benzonase (Novagen Cat#70746-3)) were added per 1 L of original culture. The suspension was stirred for 1 hour at 4° C. Debris was removed by centrifugation for 30 minutes at 39,800×g at 4° C. The supernatant was decanted into a 500 mL beaker and 10 ml of a 50% slurry of Qiagen Ni-NTA Agarose (Cat#30250) that had been pre-equilibrated in 50 mM Tris-HCl pH 7.8, 500 mM NaCl, 10% Glycerol, 10 mM Imidazole, and 10 mM Methionine, were added and stirred for 30 minutes at 4° C. The sample was then poured into a drip column at 4° C. and washed with 10 column volumes of 50 mM Tris-HCl pH 7.8, 500 mM NaCl, 10% Glycerol, 10 mM Imidazole, and 10 mM Methionine. The protein was eluted using a step gradient with two column volumes each of the same buffer containing 50 mM, and 500 mM Imidazole, sequentially. The 6× Histidine tag was cleaved overnight using 40 units of TEV protease (Invitrogen Cat# 10127017) per 1 mg of protein while dialyzing in 50 mM Tris-HCl pH 7.8, 500 mM NaCl, 10% Glycerol, 10 mM Imidazole, and 10 mM Methionine at 4° C. The 6× Histidine tag was removed by passing the sample over a Pharmacia 5 ml IMAC column (Cat# 17-0409-01) charged with Nickel and equilibrated in 50 mM Tris-HCl pH 7.8, 500 mM NaCl, 10% Glycerol, 10 mM Imidazole, and 10 mM Methionine. The cleaved protein eluted in the flow-through, whereas the uncleaved protein and the His-tag remained bound to the Ni-column. The cleaved protein fractions were combined and concentrated using spin concentrators. Further purification was done by gel filtration chromatography on an Amersham Biosciences HiLoad 16/60 Superdex 200 prep grade (Cat# 17-1069-01) equilibrated in 25 mM Tris-HCl pH 7.5, 150 mM NaCl, and 5 mM DTT. The cleanest fractions were combined and concentrated to ˜15 mg/ml by centrifugation in an Amicon Ultra-15 10,000 Da MWCO centrifugal filter unit (Cat# UFC901024).

cAbl Luminescence-based Enzyme Assay

Materials: Abl substrate peptide=EAIYAAPFAKKK-OH (Biopeptide, San Diego, Calif.), ATP (Sigma Cat#A-3377, FW=551), HEPES buffer, pH 7.5, Bovine serum albumin (BSA) (Roche 92423420), MgCl₂, Staurosporine (Streptomyces sp. Sigma Cat#85660-1MG), white Costar 384-well flat -bottom plate (VWR Cat#29444-088), Abl kinase (see below), Kinase-Glo™ (Promega Cat#V6712).

Stock Solutions: 10 mM Abl substrate peptide (13.4 mg/ml in miliQH₂O) stored at −20° C.; 100 mM HEPES buffer, pH 7.5 (5 ml 1M stock+45ml miliQH₂O); 10 mM ATP (5.51 mg/ml in dH₂O) stored at −20° C. (diluted 50 μl into total of 10 ml miliQH₂O daily =50 μM ATP working stock); 1% BSA (1 g BSA in 100 ml 0.1 M HEPES, pH 7.5, stored at −20° C.), 100 μM MgCl₂; 200 μM Staurosporine, 2× Kinase-Glo™ reagent (made fresh or stored at −20° C.).

Standard Assay Setup for 384-well format (20 μl kinase reaction, 40 μl detection reaction): 10 mM MgCl₂; 100 μM Abl substrate peptide; 0.1% BSA; 1 μl test compound (in DMSO); 0.4 μg/ml Abl kinase domain; 10 μM ATP; 100 mM HEPES buffer. Positive controls contained DMSO with no test compound. Negative controls contained 10 μM staurosporine.

The kinase reactions were initiated at time t=0 by the addition of ATP. Kinase reactions were incubated at 21° C. for 30 min, then 20 μl of Kinase-Glo™ reagent were added to each well to quench the kinase reaction and initiate the luminescence reaction. After a 20 min incubation at 21° C., the luminescence was detected in a plate-reading luminometer.

MET Luminescence-based Enzyme Assay

Materials: Poly Glu-Tyr (4:1) substrate (Sigma Cat# P-0275), ATP (Sigma Cat#A-3377, FW=551), HEPES buffer, pH 7.5, Bovine serum albumin (BSA) (Roche 92423420), MgCl₂, Staurosporine (Streptomyces sp. Sigma Cat#85660-1MG), white Costar 384-well flat-bottom plate (VWR Cat#29444-088). MET kinase (see below), Kinase-Glo™ (Promega Cat#V6712).

Stock Solutions: 10 mg/ml poly Glu-Tyr in water, stored at −20° C.; 100 mM HEPES buffer, pH 7.5 (5 ml 1M stock+45 ml miliQH₂O); 10 mM ATP (5.51 mg/ml in dH₂O) stored at −20° C. (diluted 50 μl into total of 10 ml miliQH₂O daily=50 μM ATP working stock); 1% BSA (1 g BSA in 100 ml 0.1 M HEPES, pH 7.5, stored at −20° C.), 100 mM MgCl₂; 200 μM Staurosporine, 2× Kinase-Glo™ reagent (made fresh or stored at −20° C.).

Standard Assay Setup for 384-well format (20 μl kinase reaction, 40 μl detection reaction): 10 mM MgCl₂; 0.3 mg/ml poly Glu-Tyr; 0.1% BSA; 1 μl test compound (in DMSO); 0.4 μg/ml MET kinase; 10 μM ATP; 100 mM HEPES buffer. Positive controls contained DMSO with no test compound. Negative controls contained 10 μM staurosporine. The kinase reactions were initiated at time t=0 by the addition of ATP. Kinase reactions were incubated at 21° C. for 60 min, then 20 μl of Kinase-Glo™ reagent were added to each well to quench the kinase reaction and initiate the luminescence reaction. After a 20 min incubation at 21° C., the luminescence was detected in a plate-reading luminometer.

AurA Luminescence-based Enzyme Assay

Materials: Kemptide peptide substrate=LRRASLG (Biopeptide, San Diego, Calif.), ATP (Sigma Cat#A-3377, FW=551), HEPES buffer, pH 7.5, 10% Brij 35 (Calbiochem Cat# 203728), MgCl₂, Staurosporine (Streptomyces sp. Sigma Cat#85660-1MG), white Costar 384-well flat -bottom plate (VWR Cat#29444-088), Autophosphorylated AurA kinase (see below), Kinase-Glo™ (Promega Cat#V6712).

Stock Solutions: 10 mM Kemptide peptide (7.72 mg/ml in water), stored at −20° C.; 100 mM HEPES buffer +0.015% Brij 35, pH 7.5 (5 ml 1M HEPES stock +75 μL 10% Brij 35+45 ml miliQH₂O); 10 mM ATP (5.51 mg/ml in dH₂O) stored at −20° C. (diluted 50 μl into total of 10 ml miliQH₂O daily=50 μM ATP working stock); 100 mM MgCl₂; 200 μM Staurosporine, 2× Kinase-Glo™ reagent (made fresh or stored at −20° C.).

AurA Autophosphorylation Reaction: ATP and MgCl₂ were added to 1-5 mg/ml AurA at final concentrations of 10 mM and 100 mM, respectively. The autophosphorylation reaction was incubated at 21° C. for 2-3 h. The reaction was stopped by the addition of EDTA to a final concentration of 50 mM, and samples were flash frozen with liquid N₂ and stored at −80° C.

Standard Assay Setup for 384-well format (20 μl kinase reaction, 40 μL detection reaction): 10 mM MgCl₂; 0.2mM Kemptide peptide; 1 μl test compound (in DMSO); 0.3 μg/ml Autophosphorylated AurA kinase; 10 μM ATP; 100 mM HEPES+0.015% Brij buffer. Positive controls contained DMSO with no test compound. Negative controls contained 5 μM staurosporine. The kinase reactions were initiated at time t=0 by the addition of ATP. Kinase reactions were incubated at 21° C. for 45 min, then 20,ul of Kinase-Glo™ reagent were added to each well to quench the kinase reaction and initiate the luminescence reaction. After a 20 min incubation at 21° C., the luminescence was detected in a plate-reading luminometer.

PDK1 Luminescence-based Enzyme Assay

Materials: PDKtide peptide substrate=KTFCGTPEYLAPEVRREPRILSEEEQEMFRDFDYIADWC (Upstate Cat# 12-401), ATP (Sigma Cat#A-3377, FW=551), HEPES buffer, pH 7.5, 10% Brij 35 (Calbiochem Cat#203728), MgCl₂, Staurosporine (Streptomyces sp. Sigma Cat#85660-1MG), white Costar 384-well flat-bottom plate (VWR Cat#29444-088), PDK1 kinase (see below), Kinase-Glo™ (Promega Cat#V6712).

Stock Solutions: 1 mM PDKtide substrate (1 mg in 200 μl, as supplied by Upstate), stored at −20° C.; 100 mM HEPES buffer, pH 7.5 (5 ml 1M HEPES stock+45 ml miliQH₂O); 10 mM ATP (5.51 mg/ml in dH₂O) stored at −20° C. (diluted 25 μl into total of 10 ml miliQH₂O daily=25 μM ATP working stock); 100 mM MgCl₂; 10% Brij 35 stored at 2-8° C.; 200 μM Staurosporine, 2× Kinase-Glo™ reagent (made fresh or stored at −20° C.).

Standard Assay Setup for 384-well format (20 μl kinase reaction, 40 μl detection reaction): 10 mM MgCl₂; 0.01 mM PDKtide; 1 μl test compound (in DMSO); 0.1 μg/ml PDK1 kinase; 5 μM ATP; 10 mM MgCl₂; 100 mM HEPES+0.01% Brij buffer. Positive controls contained DMSO with no test compound. Negative controls contained 10 μM staurosporine. The kinase reactions were initiated at time t=0 by the addition of ATP. Kinase reactions were incubated at 21° C. for 40 min, then 20 μl of Kinase-Glo™ reagent were added to each well to quench the kinase reaction and initiate the luminescence reaction. After a 20 min incubation at 21° C., the luminescence was detected in a plate-reading luminometer.

Some compounds of the invention inhibit PDK1 kinase with IC₅₀s below 5 uM.

³³PanQuinase Activity Assay (ProQuinase GmbH) and SelectScreen™ Kinase Profiling (Invitrogen Corp.)

³³PanQuinase Activity Assay is a proprietary, radioisotopic protein kinase assay developed by ProQuinase GmbH, Freiburg, Germany. Details on assay conditions can be found on the company's website.

SelectScreen™ is a trademark screening assay protocol for kinases developed by Invitrogen Corporation, Madison, Wis. Details on assay conditions can be found on the company's website.

Some compounds of the invention inhibit kinases such as BRAF, FLT3, FLT4, CDKs, CSF1R, FGFR2, KDR, RET, TRKC, VEGFR2, and AurB with IC₅₀s below 5 uM.

Kinase Activity Table

| | T315I | | | CDK4/ |
|---|---|---|---|---|
| | Abl | AurA | Met | CycD1 |
| Compound | IC50 | IC50 | IC50 | IC50* |
| Cyclopropanecarboxylic acid {3-[5- | A | A | C | A |
| (2,6-dichloro-phenyl)-imidazol-1-yl]- | | | | |
| 1H-pyrazolo[3,4-d]thiazol-5-yl}- | | | | |
| amide | | | | |
| Cyclopropanecarboxylic acid {3-[5- | A | A | B | A |
| (2-chloro-phenyl)-imidazol-1-yl]-1H- | | | | |
| pyrazolo[3,4-d]thiazol-5-yl}-amide | | | | |
| Cyclopropanecarboxylic acid {3-[5- | A | B | B | A |
| (4-carbamoylmethoxy-2-chloro- | | | | |
| phenyl)-imidazol-1-yl]-1H- | | | | |
| pyrazolo[3,4-d]thiazol-5-yl}-amide | | | | |
| 1-{3-[5-(2-Chloro-phenyl)-imidazol- | | C | D | B |
| 1-yl]-1H-pyrazolo[3,4-d]thiazol-5-yl}- | | | | |
| 3-(1H-pyrazol-3-yl)-urea | | | | |
| Cyclopropanecarboxylic acid {3-[5- | A | C | C | A |
| (2,3-difluoro-phenyl)-imidazol-1-yl]- | | | | |
| 1H-pyrazolo[3,4-d]thiazol-5-yl}- | | | | |
| amide | | | | |
| Cyclopropanecarboxylic acid {3-[5- | A | A | C | |
| (2,3,6-trichloro-phenyl)-imidazol-1- | | | | |
| yl]-1H-pyrazolo[3,4-d]thiazol-5-yl}- | | | | |
| amide | | | | |
| Cyclopropanecarboxylic acid {3-[5- | | B | C | C |
| (2,3,5-trichloro-phenyl)-imidazol-1- | | | | |
| yl]-1H-pyrazolo[3,4-d]thiazol-5-yl}- | | | | |
| amide | | | | |
| Cyclopropanecarboxylic acid {3-[5- | | B | D | |
| (5-fluoro-2-methanesulfonyl-phenyl)- | | | | |
| imidazol-1-yl]-1H-pyrazolo[3,4- | | | | |
| d]thiazol-5-yl}-amide | | | | |
| Cyclopropanecarboxylic acid [3-(5- | A | D | D | |
| pyridin-2-yl-imidazol-1-yl)-1H- | | | | |
| pyrazolo[3,4-d]thiazol-5-yl]-amide | | | | |
| Cyclopropanecarboxylic acid [3-(5- | B | C | D | |
| phenyl-imidazol-1-yl)-1H- | | | | |
| pyrazolo[3,4-d]thiazol-5-yl]-amide | | | | |
| Cyclopropanecarboxylic acid [3-(5- | C | C | C | |
| naphthalen-2-yl-imidazol-1-yl)-1H- | | | | |
| pyrazolo[3,4-d]thiazol-5-yl]-amide | | | | |
| Cyclopropanecarboxylic acid {3-[5- | | D | B | |
| (2-chloro-6-methoxy-quinolin-3-yl)- | | | | |
| imidazol-1-yl]-1H-pyrazolo[3,4- | | | | |
| d]thiazol-5-yl}-amide | | | | |

A: IC₅₀ < 100 nM
B: 100 nM < IC₅₀ < 1 uM
C: 1 uM < IC₅₀ < 10 uM
D: IC₅₀ > 10 uM
*ProQuinase Assay

### Example 3

### Cell Assays

GTL16 cells were maintained in DMEM Medium supplemented with 10% fetal bovine serum (FBS) 2 mM L-Glutamine and 100 units penicillin/100 μg streptomycin, at 37° C. in 5% CO₂.

HCT116 cells were maintained in McCoy's 5a Medium supplemented with 10% fetal bovine serum (FBS) 2 mM L-Glutamine and 100 units penicillin/100 μg streptomycin, at 37° C. in 5% CO₂.

Ba/F3 cells were maintained in RPMI 1640 supplemented with 10% FBS, penicillin/streptomycin and 5 ng/ml recombinant mouse IL-3.

Compounds were tested in the following assays in duplicate.

Cell Survival Assays

96-well XTT assay (GLT16 cells): One day prior to assay the growth media was aspirated off and assay media was added to cells. On the day of the assay, the cells were grown in assay media containing various concentrations of compounds (duplicates) on a 96-well flat bottom plate for 72 hours at 37° C. in 5% CO₂. The starting cell number was 5000 cells per well and volume was 120 μl. At the end of the 72-hour incubation, 40 μl of XTT labeling mixture (50:1 solution of sodium 3′-[1-(phenylaminocarbonyl)-3,4-tetrazolium]-bis (4-methoxy-6-nitro) benzene sulfonic acid hydrate and Electron-coupling reagent: PMS (N-methyl dibenzopyrazine methyl sulfate) were added to each well of the plate. After an additional 5 hours of incubation at 37° C., the absorbance reading at 450 nm with a background correction of 650 nm was measured with a spectrophotometer.

96-well XTT assay (HCT116 cells): Cells were grown in growth media containing various concentrations of compounds (duplicates) on a 96-well flat bottom plate for 72 hours at 37° C. in 5% CO₂. The starting cell number was 5000 cells per well and volume was 120 μl. At the end of the 72-hour incubation, 40 μl of XTT labeling mixture (50:1 solution of sodium 3′-[1-(phenylaminocarbonyl)-3,4-tetrazolium]-bis (4-methoxy-6-nitro) benzene sulfonic acid hydrate and Electron-coupling reagent: PMS (N-methyl dibenzopyrazine methyl sulfate) were added to each well of the plate. After an additional 2-6 hours of incubation at 37° C., the absorbance reading at 650 nm was measured with a spectrophotometer.

96-well XTT assay (Ba/F3 cells): Cells were grown in growth media containing various concentrations of compounds (duplicates) on a 96-well plate for 72 hours at 37° C. The starting cell number was 5000-8000 cells per well and volume was 120 μl. At the end of the 72-hour incubation, 40 μl of XTT labeling mixture (50:1 solution of sodium 3′-[1-(phenylamino-carbonyl)-3,4-tetrazolium]-bis (4-methoxy-6-nitro) benzene sulfonic acid hydrate and Electron-coupling reagent: PMS (N-methyl dibenzopyrazine methyl sulfate) were added to each well of the plate. After an additional 2-6 hours of incubation at 37° C., the absorbance reading at 405 nm with background correction at 650 nm was measured with a spectrophotometer.

Phosphorylation Assays

Met phosphorylation assay: GTL16 cells were plated out at 1×1ˆ6 cells per 60×15 mm dish (Falcon) in 3 mL of assay media. The following day compound at various concentrations were added in assay media and incubated for 1 hour at 37° C. 5% CO2. After 1 hour the media was aspirated, and the cells were washed once with 1×PBS. The PBS was aspirated and the cells were harvested in 100 μL of modified RIPA lysis buffer (Tris.Cl pH 7.4, 1% NP-40, 5 mM EDTA, 5 mM NaPP, 5 mM NaF, 150 mM NaCl, Protease inhibitor cocktail (Sigma), 1 mM PMSF, 2 mM NaVO₄) and transferred to a 1.7 mL eppendorf tube and incubated on ice for 15 minutes. After lysis, the tubes were centrifuged (10 minutes, 14,000 g, 4° C.). Lysates were then transferred to a fresh eppendorf tube. The samples were diluted 1:2 (250,000 cells/tube) with 2×SDS PAGE loading buffer and heated for 5 minutes at 98° C. The lysates were separated on a NuPage 4-12% Bis-Tris Gel 1.0 mm×12 well (Invitrogen), at 200V, 400 mA for approximately 40 minutes. The samples were then transferred to a 0.45 micron Nitrocellulose membrane Filter Paper Sandwich (Invitrogen) for 1 hour at 75V, 400 mA. After transferring, the membranes were placed in blocking buffer for 1 hour at room temperature with gentle rocking. The blocking buffer was removed and a 1:500 dilution of anti-Phospho-Met (Tyr1234/1235) antibody (Cell Signaling Technologies Cat. # 3126L) in 5% BSA, 0.05% Tween 20 in 1×PBS was added and the blots were incubated overnight at room temperature. The following day the blots were washed three times with 1×PBS, 0.1% Tween20. A 1:3000 dilution of HRP conjugated goat anti-rabbit antibody (Jackson ImmunoResearch Laboratories Cat. # 111-035-003) in blocking buffer, was added and incubated for 1 hr at room temperature with gentle rocking. The blot was wash 3 times in PBS, 0.1% Tween20 and visualized by chemiluminescence with SuperSignal West Pico Chemiluminescent Substrate (Pierce #34078).

Histone-H3 phosphorylation assay: HCT116 cells were plated out at 1×10ˆ6 cells per 60×15 mm dish (Falcon) in 3 mL of growth media (McCoy's 5A Media, 10% FBS, 1% pen-strep) and incubated overnight (37° C. 5% CO2). The next day compound was added and incubated for 1 hr (37° C. 5% CO2). After 1 hr, the cells were washed once with 1×PBS, and then lysed directly on the plate with 100 μL of lysis buffer (125 mM Tris HCl pH 6.8 and 2×SDS loading buffer) and transferred to a 1.7 mL eppendorf tube and put on ice. The samples were sonicated for approximately 5 seconds and were put in a 95° C. heat block for 3 minutes. After heating, the samples were loaded on a NuPage 4-12% Bis-Tris Gel (Invitrogen), followed by electrophoretic transfer to 0.45 μm nitrocellulose membranes (Invitrogen). After transferring, the membranes were placed in Qiagen blocking buffer with 0.1% Tween for 1 hour at room temperature with gentle rocking. Anti-phospho-Histone H3 (Ser10) antibody (Upstate #06-570), was diluted 1:250 in blocking buffer and was added to the blots and incubated for 1 hour at room temperature. The blot was then washed three times with 1×PBS+0.1% Tween20. Goat-anti Rabbit HRP secondary antibody (Jackson ImmunoResearch Laboratories, Inc. #111-035-003) was diluted 1:3000 in blocking buffer, and was then added for 1 hr at room temperature. The blot was washed three times with 1×PBS+0.1% Tween20, and visualized by chemiluminescence with SuperSignal West Pico Chemiluminescent Substrate (Pierce #34078).

Cellular Activity Table

| | | | T315I | |
|---|---|---|---|---|
| | GTL16 | HCT116 | Ba/F3 | |
| | XTT | XTT | XTT | Met |
| Compound | IC50 | IC50 | IC50 | Phosp. |
| Cyclopropanecarboxylic acid {3- | A | B | B | C |
| [5-(2,6-dichloro-phenyl)- | | | | |
| imidazol-1-yl]-1H-pyrazolo[3,4- | | | | |
| d]thiazol-5-yl}-amide | | | | |
| Cyclopropanecarboxylic acid {3- | B | B | B | C |
| [5-(2-chloro-phenyl)-imidazol-1- | | | | |
| yl]-1H-pyrazolo[3,4-d]thiazol-5- | | | | |
| yl}-amide | | | | |
| 1-{3-[5-(2-Chloro-phenyl)- | A | B | | D |
| imidazol-1-yl]-1H-pyrazolo[3,4- | | | | |
| d]thiazol-5-yl}-3-(1H-pyrazol-3- | | | | |
| yl)-urea | | | | |
| Cyclopropanecarboxylic acid {3- | C | B | B | D |
| [5-(2,3-difluoro-phenyl)- | | | | |
| imidazol-1-yl]-1H-pyrazolo[3,4- | | | | |
| d]thiazol-5-yl}-amide | | | | |
| Cyclopropanecarboxylic acid {3- | B | C | B | |
| [5-(2,3,6-trichloro-phenyl)- | | | | |
| imidazol-1-yl]-1H-pyrazolo[3,4- | | | | |
| d]thiazol-5-yl}-amide | | | | |
| Cyclopropanecarboxylic acid [3- | | B | C | |
| (5-pyridin-2-yl-imidazol-1-yl)- | | | | |
| 1H-pyrazolo[3,4-d]thiazol-5-yl]- | | | | |
| amide | | | | |
| Cyclopropanecarboxylic acid [3- | C | B | B | |
| (5-phenyl-imidazol-1-yl)-1H- | | | | |
| pyrazolo[3,4-d]thiazol-5-yl]- | | | | |
| amide | | | | |

A: IC₅₀ < 100 nM
B: 100 nM < IC₅₀ < 1 uM
C: 1 uM < IC₅₀ < 10 uM
D: IC₅₀ > 10 uM

What is claimed is:

1. A compound having the formula: 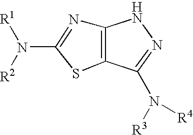
wherein
R¹ and R³ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
R² and R⁴ are independently —C(X¹)R⁵, —SO₂R⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹ is independently ═N(R⁷), ═S, or ═O, wherein R⁷ is hydrogen, cyano, —NR⁸R⁹, substituted or unsubstituted alkyl, substituted or unsubstituted heteroalkyl, substituted or unsubstituted aryl, or substituted or unsubstituted heteroaryl;
R⁵ is independently —NR⁸R⁹, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is independently —NR⁸R⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein R¹ and R², R³ and R⁴, and R⁸ and R⁹ are, independently, optionally joined with the nitrogen to which they are attached to form substituted or unsubstituted heterocycloalkyl, or substituted or unsubstituted heteroaryl.

2. The compound of claim 1, wherein
R¹ and R³ are independently hydrogen, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl;
R² and R⁴ are independently —C(X¹)R⁵, —SO₂R⁶, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl;
X¹ is independently ═N(R⁷), ═S, or ═O, wherein R⁷ is hydrogen, cyano, —NR⁸R⁹, R¹¹-substituted or unsubstituted alkyl, R¹¹-substituted or unsubstituted heteroalkyl, R¹¹-substituted or unsubstituted aryl, or R¹¹-substituted or unsubstituted heteroaryl;
R⁵ is independently —NR⁸R⁹, —OR¹⁰, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl;
R⁶ is independently —NR⁸R⁹, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl, or —NR⁸R⁹;
R⁸ and R⁹ are independently hydrogen, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl;
R¹⁰ is independently R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl;
wherein R¹ and R², R³ and R⁴, and R⁸ and R⁹ are, independently, independently, optionally joined with the nitrogen to which they are attached to form R¹¹-substituted or unsubstituted heterocycloalkyl, or R¹¹-substituted or unsubstituted heteroaryl;
wherein R¹¹ is independently halogen, -L¹-C(X²)R¹², -L¹-OR¹³, -L¹-NR¹⁴R¹⁵, -L¹-S(O)ₘR¹⁶, —CN, —NO₂, —CF₃,
(1) unsubstituted C₃-C₇ cycloalkyl;
(2) unsubstituted 3 to 7 membered heterocycloalkyl;
(3) unsubstituted heteroaryl;
(4) unsubstituted aryl;
(5) substituted C₃-C₇ cycloalkyl;
(6) substituted 3 to 7 membered heterocycloalkyl;
(7) substituted aryl;
(8) substituted heteroaryl;
(9) unsubstituted C₁-C₂₀ alkyl;
(10) unsubstituted 2 to 20 membered heteroalkyl;
(11) substituted C₁-C₂₀ alkyl; or
(12) substituted 2 to 20 membered heteroalkyl wherein
(5), (6), (11), and (12) are independently substituted with an oxo, —OH, —CF₃, —COOH, cyano, halogen, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, R¹⁸-substituted or unsubstituted heteroaryl, -L¹-C(X²)R¹², -L¹-OR³, -L¹-NR¹⁴R¹⁵, or -L¹-S(O)ₘR¹⁶,
(7) and (8) are independently substituted with an —OH, —CF₃, —COOH, cyano, halogen, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, R¹⁸-substituted or unsubstituted heteroaryl, -L¹-C(X²)R¹², -L¹-OR¹³, -L¹-NR ⁴R¹⁵, or -L¹-S(O)ₘR¹⁶, wherein
(a) X² is independently ═S, ═O, or ═NR²⁷, wherein R²⁷ is H, —CN, —NR⁸R⁹,—OR²⁸, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, or R¹⁸-substituted or unsubstituted heteroaryl, wherein R²⁸ is hydrogen or R¹⁷-substituted or unsubstituted C₁-C₁₀ alkyl,
(b) m is independently an integer from 0 to 2;
(c) R¹² is independently hydrogen, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, R¹⁸-substituted or unsubstituted heteroaryl, —OR¹⁹, or —NR²⁰R²¹, wherein
R¹⁹, R²⁰, and R²¹ are independently hydrogen, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, or R¹⁸-substituted or unsubstituted heteroaryl,
wherein R²⁰ is optionally —S(O)₂R³⁰, or —C(O)R³⁰, wherein R³⁰ is R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, or R¹⁸-substituted or unsubstituted heteroaryl,
wherein R²⁰ and R²¹ are optionally joined with the nitrogen to which they are attached to form an R¹⁷-substituted or unsubstituted 3 to 7 membered heterocycloalkyl, or R¹⁸-substituted or unsubstituted heteroaryl;
(d) R¹³, R¹⁴ and R¹⁵ are independently hydrogen, —CF₃, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, R¹⁸-substituted or unsubstituted heteroaryl, —C(X³)R²², or —S(O)₂R²², wherein R¹⁴ and R¹⁵ are optionally joined with the nitrogen to which they are attached to form an R¹⁷-substituted or unsubstituted 3 to 7 membered heterocycloalkyl, or R¹⁸-substituted or unsubstituted heteroaryl, wherein
(i) X³ is independently ═S, ═O, or ═NR²³, wherein R²³ is cyano, —NR⁸R⁹, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, or R¹⁸-substituted or unsubstituted heteroaryl; and
(ii) R²² is independently R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, R¹⁸-substituted or unsubstituted heteroaryl, or —NR²⁴R²⁵,
wherein if R¹¹ is -L¹-NR¹⁴R¹⁵ and R¹⁴ or R¹⁵ is —C(X³)R²², then R²² is optionally hydrogen,
wherein R²⁴ and R²⁵ are independently hydrogen, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, or R¹⁸-substituted or unsubstituted heteroaryl, wherein R²⁴ and R²⁵ are optionally joined with the nitrogen to which they are attached to form an R¹⁷-substituted or unsubstituted 3 to 7 membered heterocycloalkyl, or R¹⁸-substituted or unsubstituted heteroaryl;
(e) R¹⁶ is independently R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered heterocycloalkyl, R¹⁸-substituted or unsubstituted aryl, R¹⁸-substituted or unsubstituted heteroaryl, or —NR²⁶R²⁷, wherein if m is 0, then R¹⁶ is optionally hydrogen, wherein
(i) R²⁶ and R²⁷ are independently hydrogen, cyano, —NR⁸R⁹, R¹⁷-substituted or unsubstituted C₁-C₁₀ alkyl, R¹⁷-substituted or unsubstituted 2 to 10 membered heteroalkyl, R¹⁷-substituted or unsubstituted C₃-C₇ cycloalkyl, R¹⁷-substituted or unsubstituted 3 to 7 membered 21-substituted or unsubstituted heteroaryl, wherein R²⁶ and R²⁷ are optionally joined with the nitrogen to which they are attached to form an R¹⁷-substituted or unsubstituted 3 to 7 membered heterocycloalkyl, or R¹⁸-substituted or unsubstituted heteroaryl
wherein R²⁶ is optionally —C(O)R³⁰;
(f) L¹ is independently a bond, unsubstituted C₁-C₁₀ alkylene, or unsubstituted heteroalkylene;
(g) R¹⁷ is independently oxo, —OH, —COOH, —CF₃, —OCF₃, —CN, amino, halogen, R²⁸-substituted or unsubstituted 2 to 10 membered alkyl, R²⁸-substituted or unsubstituted 2 to 10 membered heteroalkyl, R²⁸-substituted or unsubstituted C₃-C₇ cycloalkyl, R²⁸-substituted or unsubstituted 3 to 7 membered heterocycloalkyl, R²⁹-substituted or unsubstituted aryl, or R²⁹-substituted or unsubstituted heteroaryl;
(h) R¹⁸ is independently —OH, —COOH, amino, halogen, —CF₃, —OCF₃, —CN, R²⁸-substituted or unsubstituted 2 to 10 membered alkyl, R²⁸-substituted or unsubstituted 2 to 10 membered heteroalkyl, R²⁸ substituted or unsubstituted C₃-C₇ cycloalkyl, R²⁸-substituted or unsubstituted 3 to 7 membered heterocycloalkyl, R²⁹-substituted or unsubstituted aryl, or R²⁹-substituted or unsubstituted heteroaryl;
(i) R²⁸ is independently oxo, —OH, —COOH, amino, halogen, —CF₃, —OCF₃, —CN, unsubstituted C₁-C₁₀ alkyl, unsubstituted 2 to 10 membered heteroalkyl, unsubstituted C₃-C₇ cycloalkyl, unsubstituted 3 to 7 membered heterocycloalkyl, unsubstituted aryl, unsubstituted heteroaryl; and
(j) R²⁹ is independently —OH, —COOH, amino, halogen, —CF₃, —OCF₃, —CN, unsubstituted C₁-C₁₀ alkyl, unsubstituted 2 to 10 membered heteroalkyl, unsubstituted C₃-C₇ cycloalkyl, unsubstituted 3 to 7 membered heterocycloalkyl, unsubstituted aryl, unsubstituted heteroaryl.

3. The compound of claim 2, wherein R¹ is hydrogen.

4. The compound of claim 2, wherein R³ is hydrogen.

5. The compound of claim 2, wherein R² is —C(X¹)R⁵, R¹¹-substituted or unsubstituted alkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl, wherein X¹ is ═O.

6. The compound of claim 2, wherein R² is —C(X¹)R⁵.

7. The compound of claim 6, wherein, R⁵ is R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl.

8. The compound of claim 6, wherein, R⁵ is R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl.

9. The compound of claim 6, wherein, R⁵ is R¹¹-substituted or unsubstituted cycloalkyl.

10. The compound of claim 2, wherein R⁴ is selected from —C(X¹)R⁵, R¹¹-substituted or unsubstituted alkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl, wherein X¹ is ═O.

11. The compound of claim 2, wherein R⁴ is R¹¹-substituted or unsubstituted alkyl, wherein R¹¹ is (1), (2), (3), (4), (5), (6), (7), or (8).

12. The compound of claim 2, wherein R⁴ is selected from —C(X¹)R⁵, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl, wherein X¹ is ═O.

13. The compound of claim 12, wherein R⁴ is —C(X¹)R⁵.

14. The compound of claim 13, wherein the R⁵ of said R⁴ is R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl.

15. The compound of claim 13, wherein R⁵ of said R⁴ is R¹¹-substituted or unsubstituted heteroaryl, or R¹¹-substituted or unsubstituted aryl.

16. The compound of claim 15, wherein the R¹¹ of said R⁴ is halogen, -L¹-S(O)ₘR⁶, -L¹-OR¹³, -L¹-C(X²)R¹², -L¹-NR¹⁴R¹⁵, (3), (4), (7), or (8).

17. The compound of claim 16, wherein L¹ is a bond, or methylene.

18. The compound of claim 16, wherein m is 2.

19. The compound of claim 15, wherein the R¹¹-substituted heteroaryl of said R⁴, and the R¹¹-substituted aryl of said R⁴ are substituted at the ortho position.

20. The compound of claim 2, wherein R⁴ and R³ are joined with the nitrogen to which they are attached to form an R¹¹-substituted or unsubstituted 5-membered heteroaryl.

21. The compound of claim 2, wherein R⁴ and R³ are joined with the nitrogen to which they are attached to form an R¹¹-substituted or unsubstituted heteroaryl selected from the groups consisting of R¹¹-substituted or unsubstituted pyrrolyl, R¹¹-substituted or unsubstituted imidazolyl, R¹¹-substituted or unsubstituted pyrazolyl, and R¹¹-substituted or unsubstituted triazolyl.

22. The compound of claim 21, wherein R⁴ and R³ are joined with the nitrogen to which they are attached to form an R¹¹-substituted or unsubstituted [1,2,3] triazolyl, R¹¹-substituted or unsubstituted [1,2,4] triazolyl, or R¹¹-substituted or unsubstituted [1,3,4] triazolyl.

23. The compound of claim 21, wherein the R¹¹ of the R¹¹-substituted or unsubstituted heteroaryl formed by said R³ and R⁴ is halogen, -L¹-S(O)ₘR¹⁶, -L¹-OR¹³, -L¹-C(X²)R¹², -L¹-NR¹⁴R¹⁵, (3), (4), (7), or (8).

24. The compound of claim 21, wherein the R¹¹ of the R¹¹-substituted or unsubstituted heteroaryl formed by said R³ and R⁴ is (7) or (8).

25. The compound of claim 24, wherein (7) and (8) are independently substituted with halogen, -L¹-OR¹³, -L¹-NR¹⁴R¹⁵, -L¹-C(X²)R¹², -L¹-S(O)ₘR¹⁶, R¹⁷-substituted or unsubstituted C₁-C₁₀ alkyl, or R¹⁸-substituted or unsubstituted heteroaryl.

26. The compound of claim 25, wherein L¹ is a bond or methylene.

27. The compound of claim 21, wherein the R¹¹-substituted heteroaryl formed by said R⁴ and R³ is substituted at the ortho position.

28. A method of modulating the activity of a protein kinase comprising contacting said protein kinase with a compound of claim 1.

29. A method of modulating the activity of a protein tyrosine kinase comprising contacting said protein tyrosine kinase with a compound of claim 1.

30. A method of modulating the activity of a receptor tyrosine kinase comprising contacting said receptor tyrosine kinase with a compound of claim 1.

31. A method of modulating the activity of a protein kinase comprising contacting said protein kinase with a compound of one of claim 1, wherein said protein kinase is Abelson tyrosine kinase, Ron receptor tyrosine kinase, Met receptor tyrosine kinase, 3-Phosphoinositide-dependent kinase 1, Aurora kinases, Cyclin-dependent kinases, nerve growth factor receptor (TRKC), Colony stimulating factor 1 receptor (CSF1R), or vascular endothelial growth factor receptor 2 (VEGFR2, KDR).

32. A method for treating cancer, allergy, asthma, inflammation, obstructive airway diseases, autoimmune diseases, metabolic diseases, viral diseases, bacterial infections, CNS diseases, obesity, hematological disorders, bone disorders, degenerative neural diseases, cardiovascular diseases, or diseases associated with angiogenesis, neovascularization, or vasculogenesis in a subject in need of such treatment, said method comprising administering to the subject a therapeutically effective amount of the compound of claim 1.

33. A pharmaceutical composition comprising a compound of claim 1 in admixture with a pharmaceutically acceptable excipient.
